(19) United States
(12) Patent Application Publication
(10) Pub. No.: US 2023/0320994 A1
(43) Pub. Date: Oct. 12, 2023

(54) FUNCTIONAL IONIZABLE PHOSPHOLIPIDS

(71) Applicant: The Board of Regents of The University of Texas System, Austin, TX (US)

(72) Inventors: Daniel J. SIEGWART, Dallas, TX (US); Shuai LIU, Dallas, TX (US); Xueliang YU, Dallas, TX (US); Qiang CHENG, Dallas, TX (US); Tuo WEI, Dallas, TX (US)

(21) Appl. No.: 18/042,390

(22) PCT Filed: Aug. 23, 2021
(86) PCT No.: PCT/US2021/047203

§ 371 Date: Feb. 21, 2023

Related U.S. Application Data
(60) Provisional application No. 63/068,944, filed on Aug. 21, 2020.

(51) Int. Cl. A61K 9/51; C07F 9/10; A61K 47/54

(52) U.S. Cl. CPC A61K 9/5123; A61K 47/544; C07F 9/106

(57) ABSTRACT

Ionizable phospholipids and compositions and methods relating thereof are provided herein. In some aspects, the ionizable phospholipids provided herein may be formulated in compositions which contain a nucleic acid and one or more helper excipients. In some aspects, these compositions may also be used to treat diseases or disorders with a therapeutic nucleic acid.

### CROSS-REFERENCE TO RELATED APPLICATION

The present application claims priority to U.S. Provisional Patent Application No. 63/068,944, filed on Aug. 21, 2020, entitled “FUNCTIONAL IONIZABLE PHOSPHOLIPIDS,” which is specifically incorporated by reference in its entirety herein.

### ACKNOWLEDGEMENT OF GOVERNMENT SUPPORT

This invention was made with government support under grant number EB025192 awarded by the National Institutes of Health. The government has certain rights in this invention.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 2 kilobytes in size, and titled 106546-697678_UTSD 3759_SequenceListing_ST25.txt.

### BACKGROUND

### Field

The present disclosure is directed to functional, synthetic ionizable phospholipids, pharmaceutical compositions comprising the disclosed synthetic ionizable phospholipids, and methods for gene editing, selective protein expression, mRNA delivery, and/or active pharmaceutical ingredient delivery in a subject, among other uses. The present disclosure is also directed to pharmaceutical compositions that include a lipid nanoparticle (LNP) loaded with a cargo.

### Discussion of Related Art

Genome editing technologies have many desirable therapeutic features that provide unique opportunities to design precision medicine therapeutics to treat human disease. However, overcoming biological barriers remains a major challenge in the effective delivery of these therapeutic agents to their desired cell and tissue targets. Currently, lipid nanoparticles (LNPs) are the most commonly used vehicles for delivery of gene editing therapeutics across the cell membrane. Although most efficacious LNPs for gene delivery rely on ionizable amines as key physiochemical parameters, many LNPs still have poor endosomal escape ability, thus rendering a significant amount of gene therapeutic cargos nonfunctional. As such, there is a need in the art for improved LNPs for delivery of gene editing therapeutics.

### SUMMARY

The following brief description is provided to indicate the nature of the subject matter disclosed herein. While certain aspects of the present disclosure are described below, the summary is not intended to limit the scope of the present disclosure.

The present disclosure is based, at least in part, on the on the identification of synthetic ionizable phospholipids for use in nanoparticles and lipid nanoparticles (LNPs). In some embodiments, a synthetic ionizable phospholipid may comprise Formula (I):

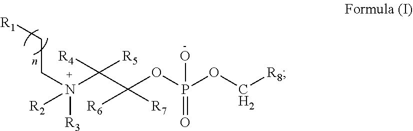

wherein: R₁ is selected from the group consisting of C2-C20 unsubstituted alkyl; C2-C20 substituted alkyl, C2-C20 unsubstituted alkenyl, C2-C20 substituted alkenyl, C2-C20 unsubstituted alkynyl, C2-C20 substituted alkynyl, C4-C20 unsubstituted cycloalkyl, and C4-C20 substituted cycloalkyl; R₂, and R₃ are independently selected from the group consisting of H, C1-C20 unsubstituted alkyl, C1-C20 substituted alkyl, C1-C20 unsubstituted alkenyl, C1-C20 substituted alkenyl, C1-C20 unsubstituted alkynyl, C1-C20 substituted alkynyl, C4-C20 unsubstituted cycloalkyl, and C4-C20 substituted cycloalkyl; R₄, R₅, R₆, and R₇ are independently selected from the group consisting of H, C1-C8 unsubstituted alkyl, C1-C8 substituted alkyl, C1-C8 unsubstituted alkenyl, C1-C8 substituted alkenyl, C1-C8 unsubstituted alkynyl, and C1-C8 substituted alkynyl; R₈ is selected from the group consisting of C3-C21 unsubstituted alkyl, C3-C21 substituted alkyl, C3-C21 unsubstituted alkenyl, C3-C21 substituted alkenyl, C3-C21 unsubstituted alkynyl, and C3-C21 substituted alkynyl; and n is an integer from 1 to 4. In some embodiments, synthetic ionizable phospholipids herein may comprise Formula (I): wherein R₁, is selected from a group consisting of C2-C16 unsubstituted alkyl; C2-C16 substituted alkyl, or C4-C12 substituted cycloalkyl; R₂, and R₃ are independently selected from a group consisting of H, C1-C16 unsubstituted alkyl, C1-C16 substituted alkyl, or C4-C16 substituted cycloalkyl; R₄, R₅, R₆, and R₇ are independently selected from a group consisting of H, C1-C4 unsubstituted alkyl, or C1-C4 substituted alkyl; R₈ is selected from C3-C18 unsubstituted alkyl; and n is an integer from 1 to 3. In some embodiments, synthetic ionizable phospholipids herein may comprise Formula (I): wherein R₁ is C2-C15 unsubstituted alkyl; R₂ and R₃ are independently selected from a group consisting of H, C1-C16 substituted alkyl, or C4-C16 substituted cycloalkyl; R₄, R₅, R₆, and R₇ are independently selected from a group consisting of H, methyl, or ethyl; R₈ is selected from C4-C16 unsubstituted alkyl; and n is an integer from 1 to 2.

In some embodiments, a synthetic ionizable phospholipid herein may comprise Formula (II):

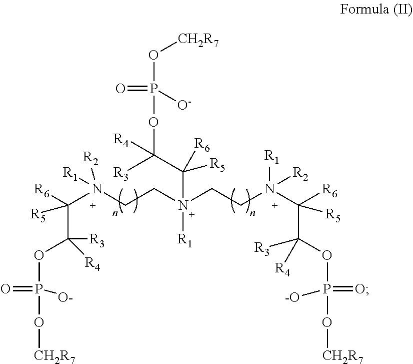

wherein R₁ and R₂ are independently selected from a group consisting of H, C1-C8 substituted alkyl, or C1-C8 unsubstituted alkyl; R₃, R₄, R₅, and R₆ are independently selected from a group consisting of H, C1-C8 unsubstituted alkyl, or C1-C8 substituted alkyl; R₇ is selected from a group consisting of C3-C21 unsubstituted alkyl or C3-C21 substituted alkyl; and n is an integer from 1 to 4. In some embodiments, synthetic ionizable phospholipids herein may comprise Formula (II): wherein R₁ and R₂ are independently selected from a group consisting of H, C1-C6 substituted alkyl, or C1-C6 unsubstituted alkyl; R₃, R₄, R₅, and R₆ are independently selected from a group consisting of H, C1-C4 unsubstituted alkyl, or C1-C4 substituted alkyl; R₇ is selected from a group consisting of C3-C18 unsubstituted alkyl or C3-C18 substituted alkyl; and n is an integer from 1 to 3. In some embodiments, synthetic ionizable phospholipids herein may comprise Formula (II): wherein R₁ and R₂ are independently selected from a group consisting of H, C1-C4 substituted alkyl, or C1-C4 unsubstituted alkyl; R₃, R₄, R₅, and R₆ are independently selected from a group consisting of H, methyl, or ethyl; R₇ is selected from a group consisting of C3-C15 unsubstituted alkyl or C3-C15 substituted alkyl; and n is an integer from 1 to 2.

In some embodiments, synthetic ionizable phospholipids herein may comprise Formula (II): wherein R₁ and R₂ are independently selected from a group consisting of H, C1-C8 substituted alkyl, C1-C8 unsubstituted alkyl, C2-C8 unsubstituted alkenyl, C2-C8 substituted alkenyl, C2-C8 unsubstituted alkynyl, or C2-C8 substituted alkynyl; R₃, R₄, R₅, and R₆ are independently selected from a group consisting of H, C1-C8 substituted alkyl, C1-C8 unsubstituted alkyl, C1-C8 unsubstituted alkenyl, C2-C8 substituted alkenyl, C2-C8 unsubstituted alkynyl, or C2-C8 substituted alkynyl; R₇ is selected from a group consisting of C3-C21 unsubstituted alkyl, C3-C21 substituted alkyl, C3-C21 unsubstituted alkenyl, C3-C21 substituted alkenyl, C3-C21 unsubstituted alkynyl, or C3-C21 substituted alkynyl; and n is an integer from 1 to 4. In some embodiments, synthetic ionizable phospholipids herein may comprise Formula (II): wherein R₁ and R₂ are independently selected from a group consisting of H, C1-C6 substituted alkyl, C1-C6 unsubstituted alkyl, C2-C6 unsubstituted alkenyl, C2-C6 substituted alkenyl, C2-C6 unsubstituted alkynyl, or C2-C6 substituted alkynyl; R₃, R₄, R₅, and R₆ are independently selected from a group consisting of H, C1-C4 unsubstituted alkyl, C1-C4 substituted alkyl, C2-C4 unsubstituted alkenyl, C2-C4 substituted alkenyl, C2-C4 unsubstituted alkynyl, or C2-C4 substituted alkynyl; R₇ is selected from a group consisting of C3-C18 unsubstituted alkyl or C3-C18 substituted alkyl; and n is an integer from 1 to 3. In some embodiments, synthetic ionizable phospholipids herein may comprise Formula (II): wherein R₁ and R₂ are independently selected from a group consisting of H, C1-C4 substituted alkyl, or C1-C4 unsubstituted alkyl; R₃, R₄, R₅, and R₆ are independently selected from a group consisting of H, methyl, or ethyl; R₇ is selected from a group consisting of C3-C15 unsubstituted alkyl or C3-C15 substituted alkyl; and n is an integer from 1 to 2.

In some embodiments, a synthetic ionizable phospholipid herein may comprise Formula (III):

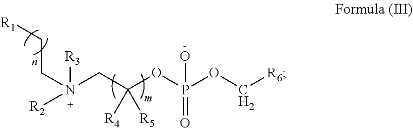

wherein: R₁ is selected from a group consisting of C2-C20 unsubstituted alkyl; C2-C20 substituted alkyl, C2-C20 unsubstituted alkenyl, C2-C20 substituted alkenyl, C2-C20 unsubstituted alkynyl, C2-C20 substituted alkynyl, C4-C20 unsubstituted cycloalkyl, or C4-C20 substituted cycloalkyl; R₂, and R₃ are independently selected from a group consisting of H, C1-C20 unsubstituted alkyl, C1-C20 substituted alkyl, C1-C20 unsubstituted alkenyl, C1-C20 substituted alkenyl, C1-C20 unsubstituted alkynyl, C1-C20 substituted alkynyl, C4-C20 unsubstituted cycloalkyl, or C4-C20 substituted cycloalkyl; R₄ and R₅ are independently selected from a group consisting of H, C1-C8 unsubstituted alkyl, C1-C8 substituted alkyl, C1-C8 unsubstituted alkenyl, C1-C8 substituted alkenyl, C1-C8 unsubstituted alkynyl, or C1-C8 substituted alkynyl; R₆ is selected from a group consisting of C3-C21 unsubstituted alkyl, C3-C21 substituted alkyl, C3-C21 unsubstituted alkenyl, C3-C21 substituted alkenyl, C3-C21 unsubstituted alkynyl, or C3-C21 substituted alkynyl; n is an integer from 1 to 4; and m is an integer from 1 to 4. In some embodiments, synthetic ionizable phospholipids herein may comprise Formula (III): wherein R₁ is selected from a group consisting of C2-C16 unsubstituted alkyl; C2-C16 substituted alkyl, C2-C16 unsubstituted alkenyl, C2-C16 substituted alkenyl, C2-C16 unsubstituted alkynyl, C2-C16 substituted alkynyl, C4-C16 unsubstituted cycloalkyl, or C4-C16 substituted cycloalkyl; R₂, and R₃ are independently selected from a group consisting of H, C1-C16 unsubstituted alkyl, C1-C16 substituted alkyl, C1-C16 unsubstituted alkenyl, C1-C16 substituted alkenyl, C1-C16 unsubstituted alkynyl, C1-C16 substituted alkynyl, C4-C16 unsubstituted cycloalkyl, or C4-C16 substituted cycloalkyl; R₄ and R₅ are independently selected from a group consisting of H, C1-C6 unsubstituted alkyl, C1-C6 substituted alkyl, C1-C6 unsubstituted alkenyl, C1-C6 substituted alkenyl, C1-C6 unsubstituted alkynyl, or C1-C6 substituted alkynyl; R₆ is selected from a group consisting of C3-C18 unsubstituted alkyl, C3-C18 substituted alkyl, C3-C18 unsubstituted alkenyl, C3-C18 substituted alkenyl, C3-C18 unsubstituted alkynyl, or C3-C18 substituted alkynyl; n is an integer from 1 to 3; and m is an integer from 1 to 3. In some embodiments, synthetic ionizable phospholipids herein may comprise Formula (III): wherein R₁ is C2-C15 unsubstituted alkyl; R₂ and R₃ are independently selected from a group consisting of H, C1-C16 substituted alkyl, or C4-C16 substituted cycloalkyl; R₄ and R₅ are independently selected from a group consisting of H, methyl, or ethyl; R₆ is selected from C4-C16 unsubstituted alkyl; n is an integer from 1 to 2; and m is an integer from 1 to 2.

In some embodiments, a synthetic ionizable phospholipid herein may comprise at least one phosphate group and at least one zwitterion, wherein the at least one zwitterion comprises a pH switchable zwitterion and/or an irreversible zwitterion. In some embodiments, a synthetic ionizable phospholipid herein may further comprise at least one tertiary amine.

In some embodiments, a synthetic ionizable phospholipid herein may further comprise a hydrophobic domain. In some embodiments, a synthetic ionizable phospholipid herein may further comprise one or more hydrophobic tails. In some embodiments, a synthetic ionizable phospholipid comprising one or more hydrophobic tails herein may consist of one hydrophobic tail. In some embodiments, a synthetic ionizable phospholipid comprising one or more hydrophobic tails herein may consist of two hydrophobic tails. In some embodiments, a synthetic ionizable phospholipid comprising one or more hydrophobic tails herein may consist of three hydrophobic tails. In some embodiments, a synthetic ionizable phospholipid comprising one or more hydrophobic tails herein may consist of four hydrophobic tails. In some embodiments, a synthetic ionizable phospholipid comprising one or more hydrophobic tails herein may consist of five hydrophobic tails. In some embodiments, a synthetic ionizable phospholipid comprising one or more hydrophobic tails herein may consist of six hydrophobic tails. In some embodiments, a synthetic ionizable phospholipid comprising one or more hydrophobic tails herein may consist of seven hydrophobic tails. In some embodiments, a synthetic ionizable phospholipid comprising one or more hydrophobic tails herein may consist of eight hydrophobic tails. In some embodiments, a synthetic ionizable phospholipid comprising one or more hydrophobic tails herein may consist of nine hydrophobic tails. In some embodiments, a synthetic ionizable phospholipid comprising one or more hydrophobic tails herein may consist of ten hydrophobic tails.

In some embodiments, a synthetic ionizable phospholipid comprising one or more hydrophobic tails herein may comprise one or more hydrophobic tails having an alkyl tail comprising an alkyl chain length of from 8 carbons to 16 carbons. In some embodiments, a synthetic ionizable phospholipid comprising one or more hydrophobic tails herein may comprise one or more hydrophobic tails having an alkyl tail comprising an alkyl chain length of from 8 carbons to 10 carbons. In some embodiments, a synthetic ionizable phospholipid comprising one or more hydrophobic tails herein may comprise one or more hydrophobic tails having an alkyl chain length of from 9 carbons to 12 carbons. In some embodiments, a synthetic ionizable phospholipid comprising one or more hydrophobic tails herein may comprise one or more hydrophobic tails having an alkyl chain length of from 13 carbons to 16 carbons. In some embodiments, a synthetic ionizable phospholipid comprising one or more hydrophobic tails herein may comprise one or more hydrophobic tails having an alkyl chain length of from 8 carbons to 16 carbons. In some embodiments, a synthetic ionizable phospholipid comprising one or more hydrophobic tails herein may comprise one or more hydrophobic tails having an alkyl tail comprising an alkyl chain length of from 8 carbons to 10 carbons. In some embodiments, a synthetic ionizable phospholipid comprising one or more hydrophobic tails herein may comprise one or more hydrophobic tails having an alkyl tail comprising an alkyl chain length of from 9 carbons to 12 carbons. In some embodiments, a synthetic ionizable phospholipid comprising one or more hydrophobic tails herein may comprise one or more hydrophobic tails having an alkyl tail comprising an alkyl chain length of from 13 carbons to 16 carbons.

In some embodiments, the present disclosure provides for pharmaceutical compositions. In some embodiments, pharmaceutical compositions herein may comprise any one of the synthetic ionizable phospholipids disclosed herein.

In some embodiments, compositions (e.g., pharmaceutical compositions, nanoparticles, LNPs) herein may further comprise at least one helper lipid. In some embodiments, compositions herein may further comprise at least one helper lipid selected from the group consisting of 1,2-dioleoyl-sn-glycero-3-phosphoethanolamine (DOPE), N-methyldioctadecylamine (MDOA), 1,2-dioleoyl-3-dimethylammonium-propane (DODAP), dimethyldioctadecylammonium bromide salt (DDAB), 1,2-dioleoyl-3-trimethylammonium-propane (DOTAP), or any combination thereof. In some embodiments, compositions herein may further comprise at least one helper lipid selected from the group consisting of zwitterionic helper lipids, ionizable cationic helper lipids, permanently cationic helper lipids, or any combination thereof. In some embodiments, compositions herein may comprise a zwitterionic helper lipid that may comprise 1,2-dioleoyl-sn-glycero-3-phosphoethanolamine (DOPE). In some embodiments, compositions herein may further comprise at least one ionizable cationic helper lipid comprising at least one lipid selected from the group consisting of N-methyldioctadecylamine (MDOA), 1,2-dioleoyl-3-dimethylammonium-propane (DODAP), and any combination thereof. In some embodiments, compositions herein may further comprise at least one permanently cationic helper lipids comprising at least one lipid selected from the group consisting of dimethyldioctadecylammonium bromide salt (DDAB), 1,2-dioleoyl-3-trimethylammonium-propane (DOTAP), and any combination thereof.

In some embodiments, compositions herein may further comprise a cholesterol and/or a cholesterol derivative. In some embodiments, compositions herein may further comprise 1,2-dimyristoyl-rac-glycero-3-methoxy(poly(ethylene glycol-2000)) (DMG-PEG2000). In some embodiments, compositions herein may further comprise one or more multi-tailed ionizable phospholipids, 1,2-dioleoyl-sn-glycero-3-phosphoethanolamine (DOPE), and cholesterol in a 55:30:45 molar ratio. In some embodiments, compositions herein may further comprise one or more multi-tailed ionizable phospholipids, N-methyldioctadecylamine (MDOA), and cholesterol in a 25:30:30 molar ratio. In some embodiments, compositions herein may further comprise one or more multi-tailed ionizable phospholipids, 1,2-dioleoyl-3-dimethylammonium-propane (DODAP), and cholesterol in a 25:30:30 molar ratio. In some embodiments, compositions herein may further comprise one or more multi-tailed ionizable phospholipids, 5A2-SC8, and cholesterol in a 25:30:30 molar ratio. In some embodiments, compositions herein may further comprise one or more multi-tailed ionizable phospholipids, dimethyldioctadecylammonium bromide salt (DDAB), and cholesterol in a 60:30:40 molar ratio. In some embodiments, compositions herein may further comprise one or more multi-tailed ionizable phospholipids, 1,2-dioleoyl-3-trimethylammonium-propane (DOTAP), and cholesterol in a 60:30:40 molar ratio. In some embodiments, compositions herein may further comprise 1,2-dimyristoyl-rac-glycero-3-methoxy(poly(ethylene glycol-2000)) (DMG-PEG2000).

In some embodiments, compositions herein may further comprise at least one cargo. In some embodiments, compositions herein may further comprise at least one cargo wherein the cargo is mRNA. In some embodiments, compositions herein may further comprise at least one cargo wherein the cargo is selected from the group consisting of an active pharmaceutical ingredient, a nucleic acid, mRNA, sgRNA, a CRISPR/Cas9 DNA sequence, a zinc finger nuclease (ZFN), transcription activator-like effector nucleases (TALENs), siRNA, miRNA, tRNA, ssDNA, base editors, peptides, proteins, CRISPR/Cas ribonucleoprotein (RNP) complexes, and any combination thereof.

In some embodiments, compositions herein may be formulated for parenteral administration. In some embodiments, compositions herein may be formulated for intravenous administration. In some embodiments, compositions herein may be formulated for topical administration.

In some embodiments, a pharmaceutical composition disclosed herein may comprise a lipid nanoparticle (LNP) loaded with a cargo, wherein the LNP comprises any ionizable phospholipid disclosed herein or one or more multi-tailed ionizable phospholipids disclosed herein, wherein the one or more multi-tailed ionizable phospholipids comprises a pH-switchable zwitterion and three hydrophobic tails.

In some embodiments, a pharmaceutical composition disclosed herein may comprise one or more multi-tailed ionizable phospholipids comprising one tertiary amine, one phosphate group, and three alkyl tails. In some embodiments, a pharmaceutical composition disclosed herein may comprise one or more multi-tailed ionizable phospholipids wherein the three hydrophobic tails or the three alkyl tails may have an alkyl chain length of from 8 carbons to 16 carbons. In some embodiments, a pharmaceutical composition disclosed herein may comprise one or more multi-tailed ionizable phospholipids wherein the three hydrophobic tails or the three alkyl tails may have an alkyl chain length of from 8 carbons to 10 carbons. In some embodiments, a pharmaceutical composition disclosed herein may comprise one or more multi-tailed ionizable phospholipids wherein the three hydrophobic tails or the three alkyl tails may have an alkyl chain length of from 9 carbons to 12 carbons. In some embodiments, a pharmaceutical composition disclosed herein may comprise one or more multi-tailed ionizable phospholipids wherein the three hydrophobic tails or the three alkyl tails may have an alkyl chain length of from 13 carbons to 16 carbons.

In some embodiments, a pharmaceutical composition disclosed herein may comprise a cargo wherein the cargo is mRNA, and wherein the pharmaceutical composition provides selective protein expression in the liver. In some embodiments, a pharmaceutical composition disclosed herein may comprise a cargo wherein the cargo is mRNA, and wherein the pharmaceutical composition provides selective protein expression in the spleen. In some embodiments, a pharmaceutical composition disclosed herein may comprise a cargo wherein the cargo is mRNA, and wherein the pharmaceutical composition provides selective protein expression in the lung. In some embodiments, a pharmaceutical composition disclosed herein may comprise a cargo wherein the cargo is mRNA, and wherein the pharmaceutical composition provides selective protein expression in the skin. In some embodiments, a pharmaceutical composition disclosed herein may comprise a cargo wherein the cargo is mRNA. In some embodiments, a pharmaceutical composition disclosed herein may comprise a cargo wherein the cargo is disposed within a core of the LNP.

In some embodiments, a pharmaceutical composition disclosed herein may comprise one or more multi-tailed ionizable phospholipids wherein the one or more multi-tailed ionizable phospholipids form a nanoparticle structure substantially encapsulating the cargo.

In some embodiments, a pharmaceutical composition disclosed herein may be for gene delivery in a subject. In some embodiments, a pharmaceutical composition disclosed herein may be for gene editing in a subject. In some embodiments, a pharmaceutical composition disclosed herein may be for comprise drug delivery in a subject. In some embodiments, a pharmaceutical composition disclosed herein may be for mRNA delivery in a subject. In some embodiments, a pharmaceutical composition disclosed herein may be for CRISPR/Cas9 gene editing in a subject. In some embodiments, a pharmaceutical composition disclosed herein may be for zinc finger nuclease (ZFN) gene editing in a subject. In some embodiments, a pharmaceutical composition disclosed herein may be for base editor gene editing in a subject. In some embodiments, a pharmaceutical composition disclosed herein may be for transcription activator-like effector nucleases (TALENs) gene editing in a subject. In some embodiments, a pharmaceutical composition disclosed herein may be for tissue-specific mRNA delivery in a subject. In some embodiments, a pharmaceutical composition disclosed herein may be for tissue-specific CRISPR/Cas9 gene editing in a subject.

In some embodiments, a pharmaceutical composition disclosed herein may comprise at least one cargo wherein the at least one cargo may be selected from the group consisting of an active pharmaceutical ingredient, a nucleic acid, mRNA, sgRNA, a CRISPR/Cas9 DNA sequence, a zinc finger nuclease (ZFN), transcription activator-like effector nucleases (TALENs), siRNA, miRNA, tRNA, ssDNA, base editors, peptides, proteins, cirRNA, CRISPR/Cas ribonucleoprotein (RNP) complexes, and any combination thereof.

In some embodiments, a pharmaceutical composition disclosed herein may comprise at least one nanoparticle. In some embodiments, a pharmaceutical composition disclosed herein may comprise at least one LNP. In some embodiments, a pharmaceutical composition disclosed herein may comprise at least one LNP wherein the at least one LNP further comprises at least one helper lipid.

In some embodiments, a pharmaceutical composition disclosed herein may comprise at least one LNP wherein the at least one LNP is a multi-component LNP for organ selective delivery, the multi-component LNP comprising multi-tailed ionizable phospholipids and one or more helper lipids. In some embodiments, a pharmaceutical composition disclosed herein may comprise at least one LNP, a cargo, one or more helper lipids, wherein the cargo is mRNA, and wherein the one or more helper lipids may enable selective protein expression in the skin, spleen, liver, and/or lung.

In some embodiments, a pharmaceutical composition disclosed herein may comprise at least one LNP, a cargo, one or more helper lipids, wherein the one or more helper lipids may be selected from the group consisting of 1,2-dioleoyl-sn-glycero-3-phosphoethanolamine (DOPE), N-methyldioctadecylamine (MDOA), 1,2-dioleoyl-3-dimethylammonium-propane (DODAP), dimethyldioctadecylammonium bromide salt (DDAB), 1,2-dioleoyl-3-trimethylammonium-propane (DOTAP), and any combination thereof. In some embodiments, a pharmaceutical composition disclosed herein may comprise at least one LNP, a cargo, one or more helper lipids, wherein the one of more helper lipids may be selected from the group consisting of zwitterionic helper lipids, ionizable cationic helper lipids, and permanently cationic helper lipids. In some embodiments, a pharmaceutical composition disclosed herein may comprise at least one LNP, a cargo, one or more helper lipids, wherein the zwitterionic helper lipids may comprise 1,2-dioleoyl-sn-glycero-3-phosphoethanolamine (DOPE). In some embodiments, a pharmaceutical composition disclosed herein may comprise at least one LNP, a cargo, one or more helper lipids, wherein the ionizable cationic helper lipids may comprise at least one lipid selected from the group consisting of N-methyldioctadecylamine (MDOA), 1,2-dioleoyl-3-dimethylammonium-propane (DODAP), 5A2-SC8, and any combination thereof. In some embodiments, a pharmaceutical composition disclosed herein may comprise at least one LNP, a cargo, one or more helper lipids, wherein the permanently cationic helper lipids may comprise at least one lipid selected from the group consisting of dimethyldioctadecylammonium bromide salt (DDAB), 1,2-dioleoyl-3-trimethylammonium-propane (DOTAP), and any combination thereof. In some embodiments, a pharmaceutical composition disclosed herein may comprise at least one LNP, a cargo, one or more helper lipids, wherein the LNP may further comprise cholesterol or a cholesterol derivative. In some embodiments, a pharmaceutical composition disclosed herein may comprise at least one LNP, a cargo, one or more helper lipids, wherein the LNP may further comprise 1,2-dimyristoyl-rac-glycero-3-methoxy(poly(ethylene glycol-2000)) (DMG-PEG2000). In some embodiments, a pharmaceutical composition disclosed herein may comprise at least one LNP, a cargo, one or more helper lipids, wherein the LNP may comprise one or more multi-tailed ionizable phospholipids, 1,2-dioleoyl-sn-glycero-3-phosphoethanolamine (DOPE), and cholesterol in a 55:30:45 molar ratio. In some embodiments, a pharmaceutical composition disclosed herein may comprise at least one LNP, a cargo, one or more helper lipids, wherein the LNP may comprise one or more multi-tailed ionizable phospholipids, N-methyldioctadecylamine (MDOA), and cholesterol in a 25:30:30 molar ratio. In some embodiments, a pharmaceutical composition disclosed herein may comprise at least one LNP, a cargo, one or more helper lipids, wherein the LNP may comprise one or more multi-tailed ionizable phospholipids, 1,2-dioleoyl-3-dimethylammonium-propane (DODAP), and cholesterol in a 25:30:30 molar ratio. In some embodiments, a pharmaceutical composition disclosed herein may comprise at least one LNP, a cargo, one or more helper lipids, wherein the LNP may comprise one or more multi-tailed ionizable phospholipids, 5A2-SC8, and cholesterol in a 25:30:30 molar ratio. In some embodiments, a pharmaceutical composition disclosed herein may comprise at least one LNP, a cargo, one or more helper lipids, wherein the LNP may comprise one or more multi-tailed ionizable phospholipids, dimethyldioctadecylammonium bromide salt (DDAB), and cholesterol in a 60:30:40 molar ratio. In some embodiments, a pharmaceutical composition disclosed herein may comprise at least one LNP, a cargo, one or more helper lipids, wherein the LNP may comprise one or more multi-tailed ionizable phospholipids, 1,2-dioleoyl-3-trimethylammonium-propane (DOTAP), and cholesterol in a 60:30:40 molar ratio. In some embodiments, a pharmaceutical composition disclosed herein may comprise at least one LNP, a cargo, one or more helper lipids, wherein the LNP may further comprise 1,2-dimyristoyl-rac-glycero-3-methoxy(poly(ethylene glycol-2000)) (DMG-PEG2000).

In some embodiments, a pharmaceutical composition disclosed herein may comprise at least one LNP, a cargo, one or more helper lipids, wherein the LNP may be capable of causing cargo release from endosomes in a target cell.

In some embodiments, a pharmaceutical composition disclosed herein may comprise at least one LNP, a cargo, one or more helper lipids, wherein the pharmaceutical composition may be formulated for parenteral administration. In some embodiments, a pharmaceutical composition disclosed herein may comprise at least one LNP, a cargo, one or more helper lipids, wherein the pharmaceutical composition may be formulated for parenteral administration, wherein the parenteral administration comprises at least one selected from the group consisting of subcutaneous administration, intracutaneous administration, intraperitoneal administration, intrathecal administration, and intramuscular administration.

In some embodiments, a pharmaceutical composition disclosed herein may comprise at least one LNP, a cargo, one or more helper lipids, wherein the pharmaceutical composition may be formulated for intravenous administration. In some embodiments, a pharmaceutical composition disclosed herein may comprise at least one LNP, a cargo, one or more helper lipids, wherein the pharmaceutical composition may be formulated for oral administration. In some embodiments, a pharmaceutical composition disclosed herein may comprise at least one LNP, a cargo, one or more helper lipids, wherein the pharmaceutical composition may be formulated for topical administration.

In some embodiments, the present disclosure provides methods of delivering an active pharmaceutical ingredient to a subject. In some embodiments, methods herein of delivering an active pharmaceutical ingredient to a subject may comprise administering to the subject a therapeutically effective amount of any pharmaceutical composition comprising a cargo as disclosed herein, wherein the cargo is an active pharmaceutical ingredient.

In some embodiments, the present disclosure provides methods of delivering mRNA or mRNA/sgRNA for gene editing in a subject to a subject. In some embodiments, methods herein of delivering mRNA or mRNA/sgRNA for gene editing in a subject to a subject may comprise administering to the subject a therapeutically effective amount of any pharmaceutical composition comprising a cargo as disclosed herein, wherein the cargo is mRNA.

In some embodiments, the present disclosure provides methods of causing selective protein expression in the liver of a subject. In some embodiments, methods of causing selective protein expression in the liver of a subject may comprise administering to the subject a therapeutically effective amount of a pharmaceutical composition herein, wherein the cargo may be mRNA and wherein the three hydrophobic tails or the three alkyl tails may have an alkyl chain length of from 9 carbons to 12 carbons. In some embodiments, the present disclosure provides methods of causing selective protein expression in the spleen of a subject. In some embodiments, methods of causing selective protein expression in the spleen of a subject may comprise administering to the subject a therapeutically effective amount of a pharmaceutical herein wherein the cargo may be mRNA and wherein the three hydrophobic tails or the three alkyl tails may have an alkyl chain length of from 13 carbons to 16 carbons. In some embodiments, the present disclosure provides methods of causing selective protein expression in the spleen, liver, skin and/or lung of a subject. In some embodiments, methods of causing selective protein expression in the spleen, liver, skin and/or lung of a subject may comprise administering to the subject a therapeutically effective amount of a pharmaceutical composition herein, wherein the cargo is mRNA.

In some embodiments, the present disclosure provides methods for gene delivery in a subject, the methods comprising administering to the subject a therapeutically effective amount of a pharmaceutical composition disclosed herein. In some embodiments, the present disclosure provides methods for gene editing in a subject, the methods comprising administering to the subject a therapeutically effective amount of a pharmaceutical composition disclosed herein. In some embodiments, the present disclosure provides methods for drug delivery in a subject, the methods comprising administering to the subject a therapeutically effective amount of a pharmaceutical composition disclosed herein. In some embodiments, the present disclosure provides methods for RNA delivery in a subject, the methods comprising administering to the subject a therapeutically effective amount of a pharmaceutical composition disclosed herein. In some embodiments, the present disclosure provides methods for CRISPR/Cas9 gene editing in a subject, the methods comprising administering to the subject a therapeutically effective amount of a pharmaceutical composition disclosed herein. In some embodiments, the present disclosure provides methods for zinc finger nuclease (ZFN) gene editing in a subject, the methods comprising administering to the subject a therapeutically effective amount of a pharmaceutical composition disclosed herein. In some embodiments, the present disclosure provides methods for base editor gene editing in a subject, the methods comprising administering to the subject a therapeutically effective amount of a pharmaceutical composition disclosed herein. In some embodiments, the present disclosure provides methods for transcription activator-like effector nucleases (TALENs) gene editing in a subject, the methods comprising administering to the subject a therapeutically effective amount of a pharmaceutical composition disclosed herein.

In some embodiments, the present disclosure provides methods tissue-specific mRNA delivery in a subject, the methods comprising administering to the subject a therapeutically effective amount of a pharmaceutical composition disclosed herein. In some embodiments, the present disclosure provides methods tissue-specific CRISPR/Cas9 gene editing in a subject, the methods comprising administering to the subject a therapeutically effective amount of a pharmaceutical composition disclosed herein.

In some embodiments, methods herein may comprise administering any composition disclosed herein to the subject by parenteral administration. In some embodiments, methods herein may comprise administering any composition disclosed herein to the subject by intravenous administration. In some embodiments, methods herein may comprise administering any composition disclosed herein to the subject by topical administration.

In some embodiments, kits are provided herein. Kits as disclosed herein may be used with any of the compositions and/or to practice any of methods disclosed herein.

Additional embodiments and features are set forth in part in the description that follows, and will become apparent to those skilled in the art upon examination of the specification or may be learned by the practice of the present disclosure. A further understanding of the nature and advantages of the present disclosure may be realized by reference to the remaining portions of the specification and the drawings, which forms a part of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description will be more fully understood with reference to the following figures and data graphs, which are presented as various embodiments of the present disclosure and should not be construed as a complete recitation of the scope of the present disclosure, wherein:

FIGS. 1A-1D depict a combinatorial library of iPhos lipids that were chemically synthesized and studied, and which led to the elucidation of a physical mechanism of action for enhanced endosomal escape. FIG. 1A depicts that efficacious iPhos lipids were composed of one ionizable amine, one phosphate group, and three hydrophobic alkyl tails; and that upon entering acidic endosomes/lysosomes, the protonation of tertiary amine induced a zwitterionic head group, which could readily insert into membranes. FIG. 1B depicts that most biological membrane phospholipids possess a zwitterion and adopt a lamellar phase; and that when iPhos lipids were mixed and inserted into the endosomal membranes, the formed conical shape by small ion pair head and multiple hydrophobic tails enabled hexagonal phase transformation. FIG. 1C depicts synthetic routes of iPhos: alkylated dioxaphospholane oxide molecules (Pm) were conjugated to amines (nA) to obtain iPhos (nAxPm); where “x” in “nAxPm” indicated the number of Pm molecules modified on one amine molecule. FIG. 1D depicts a list of 28 amines nA and 13 alkylated dioxaphospholane oxide Pm molecules used for iPhos synthesis.

FIGS. 2A-2E depict iPhos lipids with one pH-switchable zwitterion and three tails were the most efficacious for luciferase mRNA delivery in vitro. FIG. 2A depicts a heat map of luciferase expression post treatment of iPLNPs (50 ng mRNA, n=3) in IGROV1 cells. RLU>10,000 was counted for the hit rate calculation. FIG. 2B depicts representative chemical structures of iPhos with different numbers of zwitterions and tails in acidic endosomal environment. FIG. 2C depicts the relative hit rate of iPhos with a single zwitterion and multiple zwitterions. FIG. 2D depicts the relative hit rate of iPhos (1A1P4-18A1P16) with a single zwitterion and different numbers of tails. FIG. 2E depicts that among the efficient iPhos (7A1P4-13A1P16), tail length of starting amines influenced the ultimate in vitro efficacy.

FIGS. 3A-G depict model membrane studies of endosomal escape demonstrated the mechanism of iPhos lipid-mediated RNA delivery with correlation to chemical structure. FIG. 3A depicts hemolysis of 17A and 10A1P10 at pH 5.5; and that the zwitterion could significantly benefit the endosomal escape; Statistical significance: ***, P<0.001; **, P<0.01; *, P<0.05. FIG. 3B depicts hemolysis of 9A1P9 and 10A1P10 at different pHs; Statistical significance: ***, P<0.001; **, P<0.01; *, P<0.05. FIG. 3C depicts hemolysis of iPLNPs at different pHs; Statistical significance: ***, P<0.001; **, P<0.01; *, P<0.05. FIG. 3D depicts lipid fusion and membrane rupture of 10A1P10 and iPLNPs that were determined by FRET assay at pH 5.5; Statistical significance: ***, P<0.001; **, P<0.01; *, P<0.05. FIG. 3E depicts iPLNP dissociation by FRET characterization after mixing with anionic endosome mimics for 10 min at pH 5.5; and that a single zwitterion showed higher lipid fusion and iPLNP dissociation efficacy than multiple zwitterions Statistical significance: ***, P<0.001; **, P<0.01; *, P<0.05. FIG. 3F depicts 10A1P10 iPLNP dissociation of different time intervals at pH 5.5 and data are shown as average±SD; n=3. FIG. 3G depicts 10A1P10 iPLNP dissociation of different time intervals at pH 5.5; Statistical significance: ***, P<0.001; **, P<0.01; *, P<0.05. Data in FIG. 3B-G are presented as mean±s.d. (n=3 biologically independent samples).

FIGS. 4A-4D depict structure-activity studies revealing that the chemical structure and alkyl length of iPhos lipids controlled in vivo efficacy and organ selectivity. FIG. 4A depicts In vivo evaluation of 51 iPhos lipids at a low Fluc mRNA dose (0.1 mg kg⁻¹); and bioluminescence images of various organs were recorded 6 h post IV injection of C57BL/6 mice; H: Heart, Lu: lung, Li: Liver, K: Kidney, S: Spleen. FIG. 4B depicts that among the efficacious 10A1P4-12A1P16 iPhos, hydrophobic chain length at amine side dramatically influenced the in vivo mRNA delivery efficacy. FIG. 4C depicts mRNA expression in liver by iPhos with different alkyl chain length at phosphate group side; and that 9 to 12 carbon lengths were the most efficient. FIG. 4D depicts mRNA expression in spleen by iPhos with different alkyl chain length at phosphate group side; and that 13 to 16 alkyl chain lengths were the most efficient.

FIGS. 5A-5N depict iPhos is a platform technology with superiority over traditionally used phospholipids that can be applied in iPLNPs with different helper lipids to enable organ selective RNA delivery. FIG. 5A depicts a proposed structure of iPhos 9A1P9 in the acidic endosomal environment. FIG. 5B depicts images of luciferase expression in liver that were recorded (Fluc mRNA, 0.25 mg kg⁻¹) showing iPhos 9A1P9 outperformed benchmark DOPE and DSPC for mRNA delivery; H: Heart, Lu: lung, Li: Liver, K: Kidney, S: Spleen. FIG. 5C depicts quantification of luciferase expression in liver that were recorded (Fluc mRNA, 0.25 mg kg⁻¹) showing iPhos 9A1P9 outperformed benchmark DOPE and DSPC for mRNA delivery; data are shown as average±SD; n=3; statistical significance: ***, P<0.001; **, P<0.01; *, P<0.05. FIG. 5D depicts iPLNPs containing zwitterionic helper lipids that mediated mRNA expression in spleen; and in vivo evaluation showing that 9A1P9 iPLNP with helper lipid DOPE was efficient in spleen (Fluc mRNA, 0.25 mg kg⁻¹). FIG. 5E depicts iPLNPs containing zwitterionic helper lipids that mediated mRNA expression in spleen and in vivo quantification showing that 9A1P9 iPLNP with helper lipid DOPE was efficient in spleen (Fluc mRNA, 0.25 mg kg⁻¹). FIG. 5F depicts iPLNPs containing ionizable cationic helper lipids that led to mRNA translation in the liver, and organ selectivity of Fluc mRNA expression by 9A1P9 iPLNP with different ionizable cationic helper lipids as assayed (Fluc mRNA, 0.25 mg kg⁻¹ for MDOA and DODAP; 0.05 mg kg⁻¹ for 5A2-SC8). FIG. 5G depicts iPLNPs containing ionizable cationic helper lipids that led to mRNA translation in the liver and quantification of Fluc mRNA expression by 9A1P9 iPLNP with different ionizable cationic helper lipids as assayed (Fluc mRNA, 0.25 mg kg⁻¹ for MDOA and DODAP; 0.05 mg kg⁻¹ for 5A2-SC8). FIG. 5H depicts iPLNPs containing permanently cationic helper lipids that induced mRNA transfection in lung and organ images of Fluc mRNA expression by 9A1P9 iPLNP using permanently cationic helper lipids DDAB and DOTAP as evaluated (Fluc mRNA, 0.25 mg kg⁻¹). FIG. 5I depicts iPLNPs containing permanently cationic helper lipids that induced mRNA transfection in lung and quantification of Fluc mRNA expression by 9A1P9 iPLNP using permanently cationic helper lipids DDAB and DOTAP as evaluated (Fluc mRNA, 0.25 mg kg⁻¹).

FIG. 5J depicts 9A1P9 iPLNPs enabled Cre mRNA delivery selectively in liver or lung and a schematic of a Cre-LoxP mouse model that could express tdTomato by translating Cre-recombinase mRNA to Cre protein to delete the stop for mediated tdTomato expression in liver and lung, respectively (Cre mRNA, 0.25 mg kg⁻¹). FIG. 5K depicts 9A1P9-5A2-SC8 iPLNPs that enabled Cre mRNA delivery selectively in liver. FIG. 5L depicts 9A1P9-DDAB iPLNPs that enabled Cre mRNA delivery selectively in lung. FIG. 5M depicts 9A1P9-5A2-SC8 iPLNP that showed much higher mRNA delivery efficacy than positive control DLin-MC3-DMA LNPs and images of luciferase expression in liver as recorded (Fluc mRNA, 0.05 mg kg⁻¹). FIG. 5N depicts 9A1P9-5A2-SC8 iPLNP that showed much higher mRNA delivery efficacy than positive control DLin-MC3-DMA LNPs and quantification of luciferase expression in liver as recorded (Fluc mRNA, 0.05 mg kg⁻¹); data are shown as average±SD; n=3; statistical significance: ***, P<0.001; **, P<0.01; *, P<0.05.

FIGS. 6A-6N depict iPLNPs that co-delivered Cas9 mRNA and sgRNA, achieving CRISPR/Cas9 gene editing selectively in the liver and lungs. FIG. 6A depicts a schematic of co-delivery of Cas9 mRNA and sgTom1 that deleted the stop cassettes and activated tdTomato protein. FIG. 6B depicts 9A1P9-5A2-SC8 iPLNPs that enabled gene editing specifically in liver. FIG. 6C depicts post IV administration of 9A1P9-5A2-SC8 iPLNPs containing Cas9 mRNA and sgTom1 to Ai9 mice, wherein tdTomato-positive cells were observed in liver. Scale bars, 50 μm. FIG. 6D depicts 9A1P9-DDAB iPLNPs that enabled gene editing specifically in lung. FIG. 6E depicts confocal fluorescence images showing tdTomato-positive cells in lung after administration of 9A1P9-DDAB iPLNPs. Scale bars, 50 μm. FIG. 6F depicts a T7E1 assay of organ selective gene editing where 9A1P9-5A2-SC8 and 9A1P9-DDAB iPLNPs containing Cas9 mRNA and sgPTEN were IV administered into C57BL/6 mice, enabling CRISPR/Cas9 gene editing in liver and lung, respectively. For all the CRISPR/Cas9 gene editing assays, Cas9 mRNA/sgRNA weight ratio of 4:1 and total RNA dose of 0.75 mg kg⁻¹ were used. FIG. 6G depicts 9A1P9-5A2-SC8 iPLNPs (liver specific, Fluc mRNA, 0.05 mg kg−1) prepared by controlled microfluidic mixing, which resulted in decreased iPLNP sizes and preserved efficacy and organ selectivity. FIG. 6H depicts that 9A1P9-5A2-SC8 iPLNPs demonstrated small sizes and fully retained precise organ selectivity. FIG. 6I depicts whole body imaging performed 6 hours after each injection demonstrating that 9A1P9-5A2-SC8 iPLNPs (Fluc mRNA, 0.05 mg kg−1) allowed repeat dosing without loss of efficacy. FIG. 6J depicts quantification of luciferase expression performed 6 hours after each injection demonstrating that 9A1P9-5A2-SC8 iPLNPs (Fluc mRNA, 0.05 mg kg−1) allowed repeat dosing without loss of efficacy. FIGS. 6K-6N depict liver marker measurements (BUN (FIG. 6K); CREA (FIG. 6L); ATL (FIG. 6M); and AST (FIG. 6N)) demonstrating that 9A1P9-5A2-SC8 and 9A1P9-DDAB iPLNPs were well tolerated in vivo. Data are presented as mean±s.d. and statistical significance was analyzed by the two-tailed unpaired t-test: ****, P<0.0001; ***, P<0.001; **, P<0.01; *, P<0.05; NS, P>0.05. All data are from n=3 biologically independent mice.

FIGS. 7A-7M depict ¹H NMR spectra of alkylated dioxaphospholane oxide molecules P4-P16 (CDCl₃). The conversion yields of corresponding alcohols to products were 90.8% (P4; FIG. 7A), 96.8% (P5; FIG. 7B), 96.8% (P6; FIG. 7C), 96.0% (P7; FIG. 7D), 93.0% (P8; FIG. 7E), 87.0% (P9; FIG. 7F), 93.0% (P10; FIG. 7G), 95.3% (P11; FIG. 7H), 93.8% (P12; FIG. 7I), 95.3% (P13; FIG. 7K), 87.8% (P14; FIG. 7J), 92.3% (P15; FIG. 7L), 88.5% (P16; FIG. 7M), respectively. The conversion yields were calculated from integrals of peak a (4H, —OCH₂CH₂O— in Pm molecules) and peak e (3H, —CH₂CH₂CH₃ in both Pm and corresponding alcohol molecules).

FIG. 8 depicts iPhos syntheses using different amine starting materials. Amines with one primary, secondary, or tertiary amine group introduced a single zwitterion. Amines with several amine groups introduced multiple zwitterions.

FIG. 9 depicts ¹H NMR spectra of P10 (DMSO-d6) and selected iPhos (CDCl₃). The spectrum of P10 was recorded after stirring in DMSO-d6 at 70° C. and it remained almost unchanged. For the iPhos, the reaction occurred in DMSO-d6 at 70° C. for 3 days and the disappeared peak a (4.4 ppm) of methylene group post reaction indicated that almost all P10 had been consumed by the amine groups. In addition, less active P16 could also react completely with amines.

FIG. 10 depicts cell viability of IGROV1 cells treated with Fluc mRNA containing iPLNPs. The majority of iPhos showed negligible cytotoxicity.

FIG. 11 depicts ¹H NMR spectrum of 9A1P9 in CDCl₃; ¹H NMR (CDCl₃, ppm) δ 0.87 (m, 9H, —CH₂CH₂CH₂CH₃), 1.25 (m, 32H, —OCH₂CH₂(CH₂)₆CH₃ and —N(CH₂CH₂CH₂CH₂CH₂CH₂CH₂CH₃)₂), 1.55-1.84 (m, 6H, —OCH₂CH₂(CH₂)₆CH₃ and —N(CH₂CH₂CH₂CH₂CH₂CH₂CH₂CH₃)₂), 2.80 (m, 4H, —N(CH₂CH₂CH₂CH₂CH₂CH₂CH₂CH₃)₂), 3.87 (m, 2H, —NCH₂CH₂O—), 3.96-4.28 (m, 4H, —NCH₂CH₂O— and —OCH₂CH₂(CH₂)₆CH₃). ¹³C NMR (CDCl₃, ppm) δ 14.08, 14.10, 22.61, 22.66, 26.00, 27.00, 27.03, 29.16, 29.22, 29.25, 29.32, 29.45, 29.49, 29.62, 31.73, 31.77, 47.72. 31P NMR (CDCl₃, ppm) δ −0.91, 0.94. Mass calculated m/z 491.4, found [M+H]⁺ (LC-MS) m/z 492.4; in accordance with an embodiment of the present disclosure.

FIG. 12 depicts ¹H NMR spectrum of 10A1P10 in CDCl₃; ¹H NMR (CDCl₃, ppm) δ 0.87 (t, 9H, —CH₂CH₂CH₂CH₃), 1.25 (m, 42H, —OCH₂CH₂(CH₂)₇CH₃ and —N(CH₂CH₂CH₂CH₂CH₂CH₂CH₂CH₂CH₂CH₃)₂), 1.57-1.85 (m, 6H, —OCH₂CH₂(CH₂)₇CH₃ and —N(CH₂CH₂CH₂CH₂CH₂CH₂CH₂CH₂CH₂CH₃)₂), 2.81 (m, 4H, N(CH₂CH₂CH₂CH₂CH₂CH₂CH₂CH₂CH₂CH₃)₂), 3.87 (m, 2H, —NCH₂CH₂O—), 3.94-4.30 (m, 4H, —NCH₂CH₂O— and —OCH₂CH₂(CH₂)₇CH₃). ¹³C NMR (CDCl₃, ppm) δ 14.10, 22.68, 26.00, 27.01, 27.05, 29.28, 29.32, 29.35, 29.46, 29.51, 29.57, 29.62, 31.86, 31.89, 47.61. ³¹P NMR (CDCl₃, ppm) δ −1.08, 0.86. Mass calculated m/z 561.5, found [M+H]⁺ (LC-MS) m/z 563.6; in accordance with an embodiment of the present disclosure.

FIG. 13 depicts ¹H NMR spectrum of 9A1P15 in CDCl₃; a little DMSO remained in the product by column flash chromatography, but it would not influence 9A1P15 effect; ¹H NMR (CDCl₃, ppm) δ 0.86 (m, 9H, —CH₂CH₂CH₂CH₃), 1.24 (m, 44H, —OCH₂CH₂(CH₂)₁₂CH₃ and —N(CH₂CH₂CH₂CH₂CH₂CH₂CH₂CH₃)₂), 1.56-1.86 (m, 6H, —OCH₂CH₂(CH₂)₁₂CH₃ and —N(CH₂CH₂CH₂CH₂CH₂CH₂CH₂CH₃)₂), 2.81 (m, 4H, —N(CH₂CH₂CH₂CH₂CH₂CH₂CH₂CH₃)₂), 3.86 (m, 2H, —NCH₂CH₂O—), 3.94-4.30 (m, 4H, —NCH₂CH₂O— and —OCH₂CH₂(CH₂)₁₂CH₃). ¹³C NMR (CDCl₃, ppm) δ 14.07, 14.11, 22.61, 22.68, 25.87, 26.96, 27.01, 29.16, 29.19, 29.23, 29.35, 29.47, 29.65, 29.70, 31.72, 31.76, 47.49. 31P NMR (CDCl₃, ppm) δ −0.95, 0.80; mass calculated m/z 575.5, found [M+H]⁺ (LC-MS) m/z 576.6; in accordance with an embodiment of the present disclosure.

FIG. 14 depicts ¹H NMR spectrum of 10A1P16 in CDCl₃; ¹H NMR (CDCl₃, ppm) δ 0.87 (t, 9H, —CH₂CH₂CH₂CH₃), 1.25 (m, 54H, —OCH₂CH₂(CH₂)₁₃CH₃ and —N(CH₂CH₂CH₂CH₂CH₂CH₂CH₂CH₂CH₂CH₃)₂), 1.54-1.78 (m, 6H, —OCH₂CH₂(CH₂)₁₃CH₃ and —N(CH₂CH₂CH₂CH₂CH₂CH₂CH₂CH₂CH₂CH₃)₂), 2.76 (m, 4H, N(CH₂CH₂CH₂CH₂CH₂CH₂CH₂CH₂CH₂CH₃)₂), 3.85 (m, 2H, —NCH₂CH₂O—), 3.95-4.28 (m, 4H, —NCH₂CH₂O— and —OCH₂CH₂(CH₂)₁₃CH₃). ¹³C NMR (CDCl₃, ppm) δ 14.11, 22.68, 25.91, 27.10, 27.20, 29.31, 29.34, 29.56, 29.59, 29.64, 29.66, 29.72, 31.88, 31.91, 47.78. ³¹P NMR (CDCl₃, ppm) δ −1.00, 0.76; mass calculated m/z 645.6, found [M+H]⁺ (LC-MS) m/z 646.6; in accordance with an embodiment of the present disclosure.

FIGS. 15A-15C depict particle size and polydispersity Index (PDI) (FIG. 15A, zeta potential (FIG. 15B), and mRNA binding efficacy (FIG. 15C) of selected iPLNPs. 9A1P9, 10A1P10, 9A1P15 and 10A1P16 were purified and their iPLNPs showed sizes around 150 nm.

FIGS. 16A-16B depict pKa of selected iPLNPs wherein FIG. 16A depicts 9A1P9, 9A1P15, 10A1P10, 10A1P16 and FIG. 16B depicts 16A1P10 and 25A3P9.

FIGS. 17A-17C depict that different tail lengths at amine group side of iPhos influenced in vivo mRNA expression efficacy. FIG. 17A depicts bioluminescence imaging of various organs 6 hours post injection. C57BL/6 mice were IV injected by iPLNPs at 0.1 mg/kg FLuc mRNA and luminescence was quantified 6 hours post injection. FIG. 17B depicts structures of 7A1P11-11A1P11. FIG. 17C depicts effect of alkyl chain length at amine group side on mRNA delivery efficacy in vivo. Too short (4-6) and too long (>10) carbon lengths both mediated decreased efficacy in liver. Eight and ten carbon lengths trended to show high Fluc mRNA expression.

FIGS. 18A-18C depict that different tail lengths at the phosphate group side of iPhos influenced organ selective mRNA expression. FIG. 18A depicts bioluminescence imaging of various organs 6 hours post injection. C57BL/6 mice were IV injected by iPLNPs at 0.1 mg/kg FLuc mRNA. FIG. 18B depicts structures of 10A1P4-10A1P16. FIG. 18C depicts the effect of alkyl chain length at phosphate group side on organ selectivity. Short carbon lengths (4-8) showed no efficacy in vivo. Nine and ten carbon lengths trended to mediate high Fluc mRNA expression mainly in liver. Interestingly, longer carbon lengths (13-16) changed the majority of Fluc mRNA expression to spleen.

FIGS. 19A-19C depict particle size (FIG. 19A), zeta potential (FIG. 19B), and pKa (FIG. 19C) of 10A1P4-10A1P16 iPLNPs. 10A1P4-10A1P16 iPLNPs were generally negatively charged and showed sizes around 200 nm. These nanoparticles showed pKa from 6.0 to 6.5.

FIGS. 20A-20C depict in vivo evaluation of 9A1P15 and 10A1P16 iPLNPs. FIG. 20A depicts structures of 9A1P15 and 10A1P16 in acidic endosomal environment. FIG. 20B depicts C57BL/6 mice that were IV injected by iPLNPs at 0.25 mg/kg FLuc mRNA and luminescence quantified 6 hours post injection. FIG. 20C depicts quantification of luciferase expression in various organs as recorded by average radiance.

FIGS. 21A-21C depict that 9A1P9/mRNA weight ratio affected in vivo efficacy. FIG. 21A depicts a structure of 9A1P9 in acidic endosomal environment. FIG. 21B depicts evaluation of 9A1P9/mRNA with weight ratio of 9:1 (molar ratio 11622:1) and 18:1 (molar ratio 23244:1). iPhos: MDOA: chol: DMG-PEG2000 molar ratio was fixed at 25:30:30:1. C57BL/6 mice were IV injected by iPLNPs at 0.25 mg/kg FLuc mRNA and luminescence was quantified 6 hours post injection. FIG. 21C depicts quantification of luciferase expression in liver as recorded by average radiance (n=3).

FIG. 22 depicts structures of the helper lipids. Ionizable cationic lipids included MDOA, DODAP, and 5A2-SC8; permanently cationic lipids included DDAB and DOTAP. Zwitterionic lipid DOPE was used as helper lipid.

FIGS. 23A-23E depict characterization of 9A1P9 iPLNPs with different helper lipids. (FIG. 23A) Particle size, PDI, (FIG. 23B) zeta potential, (FIG. 23C) mRNA binding efficacy, (FIG. 23D) pKa and (FIG. 23E) in vitro mRNA delivery efficacy in IGROV-1 cells (25 ng mRNA) of 9A1P9 iPLNPs with different helper lipids was assessed. 9A1P9: MDOA (DODAP or 5A2-SC8): chol: DMG-PEG2000 (molar ratio)=25:30:30:1. 9A1P9: DDAB (or DOTAP): chol: DMG-PEG2000 (molar ratio)=60:30:40:0.4. 9A1P9: DOPE: chol: DMG-PEG2000 (molar ratio)=55:30:45:0.2; for all the formulations, 9A1P9: mRNA (w/w)=18:1.

FIGS. 24A-24C depict organ distribution of Cy5-mRNA. C57BL/6 mice were IV injected with PBS (FIG. 24A) or iPLNPs 9A1P9-5A2-SC8 (FIG. 24B) or 9A1P9-DDAB (FIG. 24C) at 0.25 mg/kg Cy5-mRNA and imaged after 6 hour. For iPLNP formulations, 9A1P9: 5A2-SC8: chol: DMG-PEG2000 molar ratio of 25:30:30:1 and 9A1P9: DDAB: chol: DMG-PEG2000 molar ratio of 60:30:40:0.4 were used; 9A1P9/mRNA (w/w) was fixed at 18/1.

FIGS. 25A-25B depict 9A1P9-5A2-SC8 (FIG. 25A) and 9A1P9-DDAB (FIG. 25B) iPLNPs which showed high mRNA delivery efficacy in liver and lung, respectively. 9A1P9: 5A2-SC8: chol: DMG-PEG2000 (molar ratio) of 25:30:30:1 (Fluc mRNA, 0.05 mg/kg) and 9A1P9: DDAB: chol: DMG-PEG2000 (molar ratio) of 60:30:40:0.4 (Fluc mRNA, 0.25 mg/kg) were used. 9A1P9: mRNA weight ratio was fixed at 18:1. C57BL/6 mice were IV injected by iPLNPs and imaged after 6 hours.

FIGS. 26A-26C depict kinetic analysis of Fluc protein expression by different organ selective iPLNPs. 9A1P9-5A2-SC8 (liver specific, 0.05 mg kg−1 FlucmRNA; FIG. 26A), 9A1P9-DDAB (lung specific, 0.25 mg kg−1 Fluc mRNA; FIG. 26B) and 10A1P16-MDOA (spleen specific, 0.25 mg kg−1 Fluc mRNA; FIG. 26C) iPLNPs were IV administrated to C57BL/6 mice. Organs were imaged after 3 hours, 6 hours, 12 hours, and 24 hours. Data are presented as mean±s.d. (n=3 biologically independent mice).

FIGS. 27A-27C depict 9A1P9-5A2-SC8 iPLNPs achieved ˜91% tdTomato editing in hepatocytes. FIG. 27A depicts 9A1P9-5A2-SC8 iPLNPs (Cre mRNA, 0.25 mg kg−1) that were IV administrated in Ai9 mice. After 48 hours, hepatocytes were isolated and analyzed by flow cytometry. FIG. 27B depicts DLin-MC3-DMA LNPs (Cre mRNA, 0.25 mg kg−1) that were IV injected to Ai9 mice. After 48 hours, hepatocytes were isolated and analyzed by flow cytometry. FIG. 27C depicts merged results of 9A1P9-5A2-SC8 iPLNPs and DLin-MC3-DMA LNPs. All data are from n=3 biologically independent mice.

FIG. 28 depicts the percentage of tdTomato positive lung cells. The FACS gating strategy for analysis of tdTomato expression in lung cells was used. In brief, Ghost Red 780 was utilized to distinguish live and dead cells. EpCam+ was used to define epithelial cells, CD45+ and CD31− were used to define immune cells, and CD45− and CD31+ were used to define endothelial cells. Gates for tdTomato+ in cell types were drawn based on PBS injected control Ai9 mice. Ai9 mice were IV injected by 9A1P9-DDAB iPLNPs (Cre mRNA, 0.25 mg kg−1) and tdTomato+ in given cell types was detected by flow cytometry after 48 hours. Data are presented as mean±s.d. (n=3 biologically independent mice).

FIG. 29 depicts the percentage of tdTomato positive splenic cells. The FACS gating strategy for analysis of tdTomato expression in splenic cells was used. In brief, Ghost Red 780 was utilized to distinguish live and dead cells. CD45+ was used to distinguish immune cells, then CD3+ and CD11b− were used for T cells, CD3− and CD11b+ were used for macrophage cells, and CD19+ and CD11b− were used for B cells. Gates for tdTomato+ in cell types were drawn based on PBS injected control mice. Ai9 mice were IV injected by 10A1P16-MDOA iPLNPs (Cre mRNA, 0.5 mg kg⁻¹) and tdTomato+ in given cell types was detected by flow cytometry after 48 hours. Data are presented as mean±s.d. (n=3 biologically independent mice).

FIGS. 30A-30B depict how theNanoAssemblr microfluidic mixing system can be used to decrease iPLNP size. 10A1P16-MDOA iPLNPs were prepared using the NanoAssemblr microfluidic mixing system, demonstrating small size below 100 nm. High in vivo mRNA delivery efficacy and precise organ selectivity were fully retained after decreasing iPLNP diameters (FIG. 30A). 10A1P16-MDOA iPLNPs (Fluc mRNA, 0.25 mg kg−1) mediated mRNA translation in the spleen (FIG. 30B). (n=3 biologically independent mice).

FIGS. 31A-31D depict liver marker mesurements (BUN (FIG. 31A); CREA (FIG. 31B); ATL (FIG. 31C); and AST (FIG. 31D)) demonstrating that 10A1P16-MDOA iPLNPs loading mRNA are well tolerated in vivo. 10A1P16-MDOA (spleen specific) iPLNPs were administrated intravenously (IV) to C57BL/6 mice (0.25 mg kg−1). Lipopolysaccharide (LPS, 5 mg kg−1, IP) was used as the positive control and PBS (IV) was examined as the negative control. 24 hours post injection, kidney function (BUN and CREA) and liver function (ALT and AST) were evaluated. LPS treated mice showed severe kidney and liver injury. There was no significant difference between the 10A1P16-MDOA iPLNP and PBS groups. Data are presented as mean±s.d. (n=3 biologically independent mice). Statistical significance was analyzed by the two-tailed unpaired t-test: ****, P<0.0001; ***, P<0.001; **, P<0.01; *, P<0.05.

FIGS. 32A-32B depict iPLNPs enablement of efficient delivery of pDNA and siRNA. FIG. 32A depicts 9A1P9-5A2-SC8 iPLNPs and 9A1P9-DDAB iPLNPs that were used to deliver pCMV-Luc pDNA. 12.5 ng and 25 ng pDNA per well were applied. The results were normalized to untreated cells. FIG. 32B depicts 9A1P9-5A2-SC8 iPLNPs and 9A1P9-DDAB iPLNPs that efficiently delivered siLuc (siRNA). 12.5 ng and 25 ng siRNA per well were utilized. Data are presented as mean±s.d. (n=3 biologically independent samples).

FIGS. 33A-33B depict iPLNPs enablement of efficient delivery of mRNA to subcutaneous tissues. FIG. 33A depicts post subcutaneous injection of PBS, 9A1-P9, 9A1-P15, and 10A1-P16 containing Cre Recombinase (CRE) mRNA into Ai9 mice, wherein tdTomato-positive cells were observed via IVIS 44 hours after injection. FIG. 33B depicts quantification of tdTomato expression in the subcutaneous tissues (e.g., skin) of Ai9 mice 44 hours after subcutaneous injection of PBS, 9A1-P9, 9A1-P15, and 10A1-P16 containing Cre Recombinase (CRE) mRNA.

### DETAILED DESCRIPTION

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized, and changes can be made without departing from the scope of the present disclosure. The following description is, therefore, not to be taken in a limiting sense. The scope of the present disclosure is defined only by the appended claims, along with the full scope of equivalents to which such claims are entitled.

The present disclosure is based, at least in part, on the identification of synthetic ionizable phospholipids for use in lipid nanoparticles (LNPs). Accordingly, the present disclosure provides for synthetic ionizable phospholipid compositions and methods of making thereof, LNP compositions comprising synthetic ionizable phospholipids herein and methods of making thereof, methods of using the compositions disclosed herein, and kits used in practicing the methods disclosed herein.

### I. Synthetic Ionizable Phospholipids

The present disclosure provides a new class of synthetic ionizable phospholipids. In some embodiments, a synthetic ionizable phospholipid provided herein may have Formula (I):

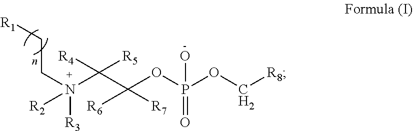

wherein: R₁ is selected from the group consisting of C2-C20 unsubstituted alkyl; C2-C20 substituted alkyl, C2-C20 unsubstituted alkenyl, C2-C20 substituted alkenyl, C2-C20 unsubstituted alkynyl, C2-C20 substituted alkynyl, C4-C20 unsubstituted cycloalkyl, and C4-C20 substituted cycloalkyl; R₂, and R₃ are independently selected from the group consisting of H, C1-C20 unsubstituted alkyl, C1-C20 substituted alkyl, C1-C20 unsubstituted alkenyl, C1-C20 substituted alkenyl, C1-C20 unsubstituted alkynyl, C1-C20 substituted alkynyl, C4-C20 unsubstituted cycloalkyl, and C4-C20 substituted cycloalkyl; R₄, R₅, R₆, and R₇ are independently selected from the group consisting of H, C1-C8 unsubstituted alkyl, C1-C8 substituted alkyl, C1-C8 unsubstituted alkenyl, C1-C8 substituted alkenyl, C1-C8 unsubstituted alkynyl, and C1-C8 substituted alkynyl; R₈ is selected from the group consisting of C3-C21 unsubstituted alkyl, C3-C21 substituted alkyl, C3-C21 unsubstituted alkenyl, C3-C21 substituted alkenyl, C3-C21 unsubstituted alkynyl, and C3-C21 substituted alkynyl; and n is an integer from 1 to 4.

In at least some instances, R₁ is selected from a group consisting of C2-C16 unsubstituted alkyl; C2-C16 substituted alkyl, or C4-C12 substituted cycloalkyl; R₂, and R₃ are independently selected from a group consisting of H, C1-C16 unsubstituted alkyl, C1-C16 substituted alkyl, or C4-C16 substituted cycloalkyl; R₄, R₅, R₆, and R₇ are independently selected from a group consisting of H, C1-C4 unsubstituted alkyl, or C1-C4 substituted alkyl; R₈ is selected from C3-C18 unsubstituted alkyl; and n is an integer from 1 to 3.

In other instances, R₁ is C2-C15 unsubstituted alkyl; R₂ and R₃ are independently selected from a group consisting of H, C1-C16 substituted alkyl, or C4-C16 substituted cycloalkyl; R₄, R₅, R₆, and R₇ are independently selected from a group consisting of H, methyl, or ethyl; R₈ is selected from C4-C16 unsubstituted alkyl; and n is an integer from 1 to 2.

In some embodiments, a synthetic ionizable phospholipid provided herein may have Formula (II):

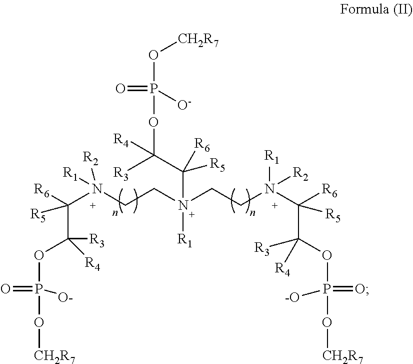

wherein R₁ and R₂ are independently selected from a group consisting of H, C1-C8 substituted alkyl, or C1-C8 unsubstituted alkyl; R₃, R₄, R₅, and R₆ are independently selected from a group consisting of H, C1-C8 unsubstituted alkyl, or C1-C8 substituted alkyl; R₇ is selected from a group consisting of C3-C21 unsubstituted alkyl or C3-C21 substituted alkyl; and n is an integer from 1 to 4.

In some instances, R₁ and R₂ are independently selected from a group consisting of H, C1-C6 substituted alkyl, or C1-C6 unsubstituted alkyl; R₃, R₄, R₅, and R₆ are independently selected from a group consisting of H, C1-C4 unsubstituted alkyl, or C1-C4 substituted alkyl; R₇ is selected from a group consisting of C3-C18 unsubstituted alkyl or C3-C18 substituted alkyl; and n is an integer from 1 to 3.

In some instances, R₁ and R₂ are independently selected from a group consisting of H, C1-C4 substituted alkyl, or C1-C4 unsubstituted alkyl; R₃, R₄, R₅, and R₆ are independently selected from a group consisting of H, methyl, or ethyl; R₇ is selected from a group consisting of C3-C15 unsubstituted alkyl or C3-C15 substituted alkyl; and n is an integer from 1 to 2.

In other instances, R₁ and R₂ are independently selected from a group consisting of H, C1-C8 substituted alkyl, C1-C8 unsubstituted alkyl, C2-C8 unsubstituted alkenyl, C2-C8 substituted alkenyl, C2-C8 unsubstituted alkynyl, or C2-C8 substituted alkynyl; R₃, R₄, R₅, and R₆ are independently selected from a group consisting of H, C1-C8 substituted alkyl, C1-C8 unsubstituted alkyl, C1-C8 unsubstituted alkenyl, C2-C8 substituted alkenyl, C2-C8 unsubstituted alkynyl, or C2-C8 substituted alkynyl; R₇ is selected from a group consisting of C3-C21 unsubstituted alkyl, C3-C21 substituted alkyl, C3-C21 unsubstituted alkenyl, C3-C21 substituted alkenyl, C3-C21 unsubstituted alkynyl, or C3-C21 substituted alkynyl; and n is an integer from 1 to 4.

In still other instances, R₁ and R₂ are independently selected from a group consisting of H, C1-C6 substituted alkyl, C1-C6 unsubstituted alkyl, C2-C6 unsubstituted alkenyl, C2-C6 substituted alkenyl, C2-C6 unsubstituted alkynyl, or C2-C6 substituted alkynyl; R₃, R₄, R₅, and R₆ are independently selected from a group consisting of H, C1-C4 unsubstituted alkyl, C1-C4 substituted alkyl, C2-C4 unsubstituted alkenyl, C2-C4 substituted alkenyl, C2-C4 unsubstituted alkynyl, or C2-C4 substituted alkynyl; R₇ is selected from a group consisting of C3-C18 unsubstituted alkyl or C3-C18 substituted alkyl; and n is an integer from 1 to 3.

In yet other instances, R₁ and R₂ are independently selected from a group consisting of H, C1-C4 substituted alkyl, or C1-C4 unsubstituted alkyl; R₃, R₄, R₅, and R₆ are independently selected from a group consisting of H, methyl, or ethyl; R₇ is selected from a group consisting of C3-C15 unsubstituted alkyl or C3-C15 substituted alkyl; and n is an integer from 1 to 2.

In some embodiments, a synthetic ionizable phospholipid provided herein may have Formula (III):

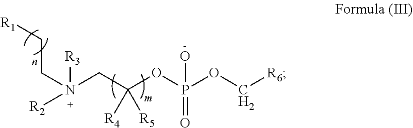

wherein: R₁ is selected from a group consisting of C2-C20 unsubstituted alkyl; C2-C20 substituted alkyl, C2-C20 unsubstituted alkenyl, C2-C20 substituted alkenyl, C2-C20 unsubstituted alkynyl, C2-C20 substituted alkynyl, C4-C20 unsubstituted cycloalkyl, or C4-C20 substituted cycloalkyl; R₂, and R₃ are independently selected from a group consisting of H, C1-C20 unsubstituted alkyl, C1-C20 substituted alkyl, C1-C20 unsubstituted alkenyl, C1-C20 substituted alkenyl, C1-C20 unsubstituted alkynyl, C1-C20 substituted alkynyl, C4-C20 unsubstituted cycloalkyl, or C4-C20 substituted cycloalkyl; R₄ and R₅ are independently selected from a group consisting of H, C1-C8 unsubstituted alkyl, C1-C8 substituted alkyl, C1-C8 unsubstituted alkenyl, C1-C8 substituted alkenyl, C1-C8 unsubstituted alkynyl, or C1-C8 substituted alkynyl; R₆ is selected from a group consisting of C3-C21 unsubstituted alkyl, C3-C21 substituted alkyl, C3-C21 unsubstituted alkenyl, C3-C21 substituted alkenyl, C3-C21 unsubstituted alkynyl, or C3-C21 substituted alkynyl; n is an integer from 1 to 4; and m is an integer from 1 to 4.

In some instances, R₁ is selected from a group consisting of C2-C16 unsubstituted alkyl; C2-C16 substituted alkyl, C2-C16 unsubstituted alkenyl, C2-C16 substituted alkenyl, C2-C16 unsubstituted alkynyl, C2-C16 substituted alkynyl, C4-C16 unsubstituted cycloalkyl, or C4-C16 substituted cycloalkyl; R₂, and R₃ are independently selected from a group consisting of H, C1-C16 unsubstituted alkyl, C1-C16 substituted alkyl, C1-C16 unsubstituted alkenyl, C1-C16 substituted alkenyl, C1-C16 unsubstituted alkynyl, C1-C16 substituted alkynyl, C4-C16 unsubstituted cycloalkyl, or C4-C16 substituted cycloalkyl; R₄ and R₅ are independently selected from a group consisting of H, C1-C6 unsubstituted alkyl, C1-C6 substituted alkyl, C1-C6 unsubstituted alkenyl, C1-C6 substituted alkenyl, C1-C6 unsubstituted alkynyl, or C1-C6 substituted alkynyl; R₆ is selected from a group consisting of C3-C18 unsubstituted alkyl, C3-C18 substituted alkyl, C3-C18 unsubstituted alkenyl, C3-C18 substituted alkenyl, C3-C18 unsubstituted alkynyl, or C3-C18 substituted alkynyl; n is an integer from 1 to 3; and m is an integer from 1 to 3.

In other instances, R₁ is C2-C15 unsubstituted alkyl; R₂ and R₃ are independently selected from a group consisting of H, C1-C16 substituted alkyl, or C4-C16 substituted cycloalkyl; R₄ and R₅ are independently selected from a group consisting of H, methyl, or ethyl; R₆ is selected from C4-C16 unsubstituted alkyl; n is an integer from 1 to 2; and m is an integer from 1 to 2.

In some embodiments, a synthetic ionizable phospholipid provided herein may have Formula (IV):

nAxPm Formula (IV);
wherein: “Pm” refers to alkylated dioxaphospholane oxide molecules, “nA” refers to amines, and “x” in “nAxPm” refers to the number of Pm molecules modified on one amine molecule. In some aspects, a synthetic ionizable phospholipid herein may have Formula (IV) wherein Pm may comprise one or more selected from:

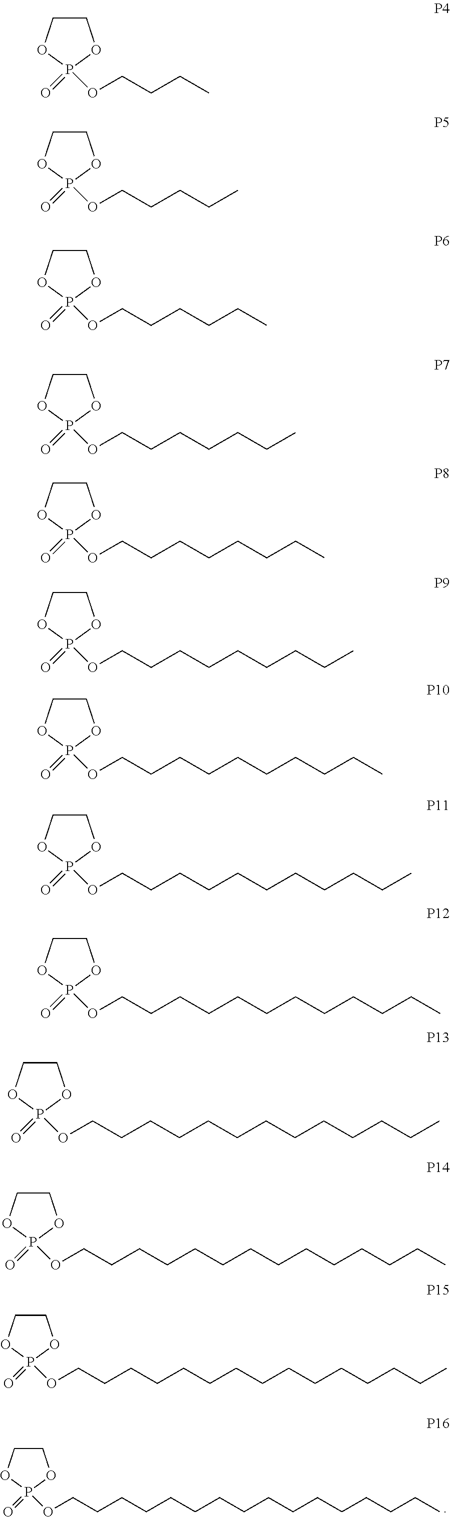

In some aspects, a synthetic ionizable phospholipid herein may have Formula (IV) wherein nA may comprise one or more selected from:

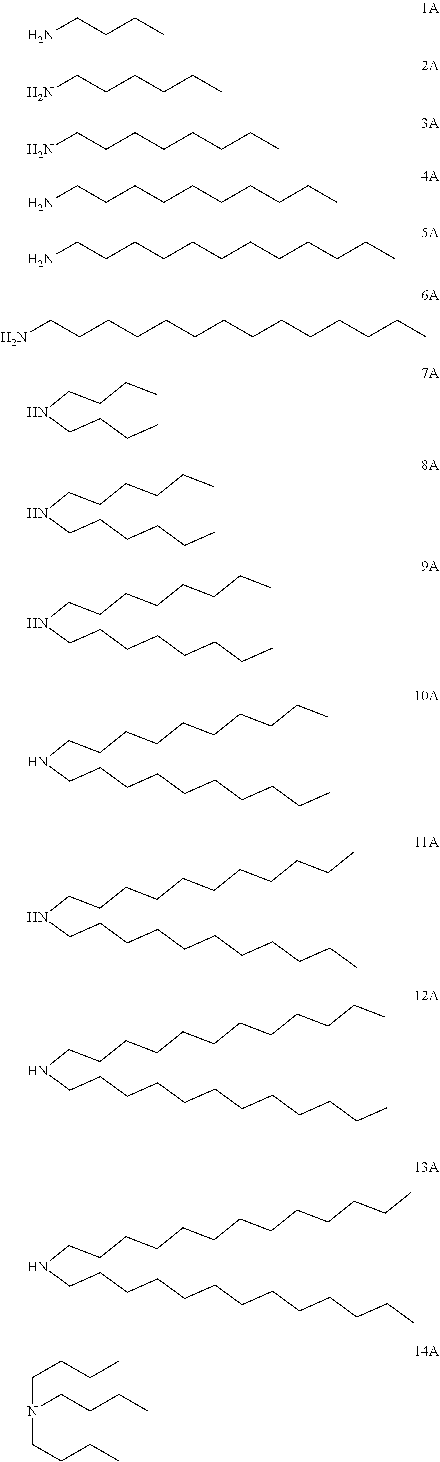

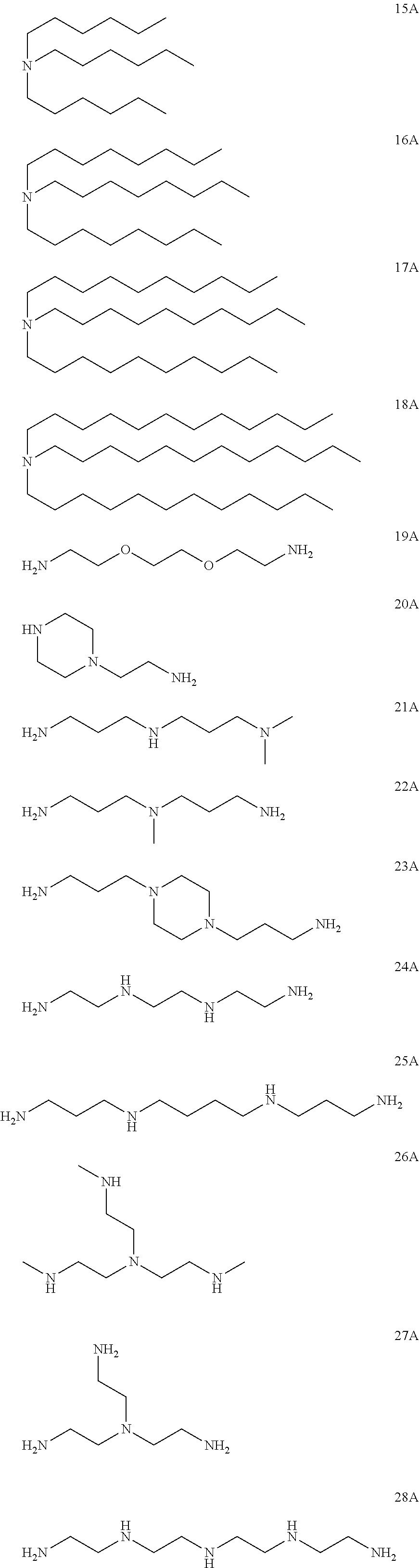

In some embodiments, a synthetic ionizable phospholipid provided herein having a nAxPm formula (IV) may include: 1AxP4, 1AxP5, 1AxP6, 1AxP7, 1AxP8, 1AxP9, 1AxP10, 1AxP11, 1AxP12, 1AxP13, 1AxP14, 1AxP15, 1AxP16, 2AxP4, 2AxP5, 2AxP6, 2AxP7, 2AxP8, 2AxP9, 2AxP10, 2AxP11, 2AxP12, 2AxP13, 2AxP14, 2AxP15, 2AxP16, 3AxP4, 3AxP5, 3AxP6, 3AxP7, 3AxP8, 3AxP9, 3AxP10, 3AxP11, 3AxP12, 3AxP13, 3AxP14, 3AxP15, 3AxP16, 4AxP4, 4AxP5, 4AxP6, 4AxP7, 4AxP8, 4AxP9, 4AxP10, 4AxP11, 4AxP12, 4AxP13, 4AxP14, 4AxP15, 4AxP16, 5AxP4, 5AxP5, 5AxP6, 5AxP7, 5AxP8, 5AxP9, 5AxP10, 5AxP11, 5AxP12, 5AxP13, 5AxP14, 5AxP15, 5AxP16, 6AxP4, 6AxP5, 6AxP6, 6AxP7, 6AxP8, 6AxP9, 6AxP10, 6AxP11, 6AxP12, 6AxP13, 6AxP14, 6AxP15, 6AxP16, 7AxP4, 7AxP5, 7AxP6, 7AxP7, 7AxP8, 7AxP9, 7AxP10, 7AxP11, 7AxP12, 7AxP13, 7AxP14, 7AxP15, 7AxP16, 8AxP4, 8AxP5, 8AxP6, 8AxP7, 8AxP8, 8AxP9, 8AxP10, 8AxP11, 8AxP12, 8AxP13, 8AxP14, 8AxP15, 8AxP16, 9AxP4, 9AxP5, 9AxP6, 9AxP7, 9AxP8, 9AxP9, 9AxP10, 9AxP11, 9AxP12, 9AxP13, 9AxP14, 9AxP15, 9AxP16, 10AxP4, 10AxP5, 10AxP6, 10AxP7, 10AxP8, 10AxP9, 10AxP10, 10AxP11, 10AxP12, 10AxP13, 10AxP14, 10AxP15, 10AxP16, 11AxP4, 11AxP5, 11AxP6, 11AxP7, 11AxP8, 11AxP9, 11AxP10, 11AxP11, 11AxP12, 11AxP13, 11AxP14, 11AxP15, 11AxP16, 12AxP4, 12AxP5, 12AxP6, 12AxP7, 12AxP8, 12AxP9, 12AxP10, 12AxP11, 12AxP12, 12AxP13, 12AxP14, 12AxP15, 12AxP16, 13AxP4, 13AxP5, 13AxP6, 13AxP7, 13AxP8, 13AxP9, 13AxP10, 13AxP11, 13AxP12, 13AxP13, 13AxP14, 13AxP15, 13AxP16, 14AxP4, 14AxP5, 14AxP6, 14AxP7, 14AxP8, 14AxP9, 14AxP10, 14AxP11, 14AxP12, 14AxP13, 14AxP14, 14AxP15, 14AxP16, 15AxP4, 15AxP5, 15AxP6, 15AxP7, 15AxP8, 15AxP9, 15AxP10, 15AxP11, 15AxP12, 15AxP13, 15AxP14, 15AxP15, 15AxP16, 16AxP4, 16AxP5, 16AxP6, 16AxP7, 16AxP8, 16AxP9, 16AxP10, 16AxP11, 16AxP12, 16AxP13, 16AxP14, 16AxP15, 16AxP16, 17AxP4, 17AxP5, 17AxP6, 17AxP7, 17AxP8, 17AxP9, 17AxP10, 17AxP11, 17AxP12, 17AxP13, 17AxP14, 17AxP15, 17AxP16, 18AxP4, 18AxP5, 18AxP6, 18AxP7, 18AxP8, 18AxP9, 18AxP10, 18AxP11, 18AxP12, 18AxP13, 18AxP14, 18AxP15, 18AxP16, 19AxP4, 19AxP5, 19AxP6, 19AxP7, 19AxP8, 19AxP9, 19AxP10, 19AxP11, 19AxP12, 19AxP13, 19AxP14, 19AxP15, 19AxP16, 20AxP4, 20AxP5, 20AxP6, 20AxP7, 20AxP8, 20AxP9, 20AxP10, 20AxP11, 20AxP12, 20AxP13, 20AxP14, 20AxP15, 20AxP16, 21AxP4, 21AxP5, 21AxP6, 21AxP7, 21AxP8, 21AxP9, 21AxP10, 21AxP11, 21AxP12, 21AxP13, 21AxP14, 21AxP15, 21AxP16, 22AxP4, 22AxP5, 22AxP6, 22AxP7, 22AxP8, 22AxP9, 22AxP10, 22AxP11, 22AxP12, 22AxP13, 22AxP14, 22AxP15, 22AxP16, 23AxP4, 23AxP5, 23AxP6, 23AxP7, 23AxP8, 23AxP9, 23AxP10, 23AxP11, 23AxP12, 23AxP13, 23AxP14, 23AxP15, 23AxP16, 24AxP4, 24AxP5, 24AxP6, 24AxP7, 24AxP8, 24AxP9, 24AxP10, 24AxP11, 24AxP12, 24AxP13, 24AxP14, 24AxP15, 24AxP16, 25AxP4, 25AxP5, 25AxP6, 25AxP7, 25AxP8, 25AxP9, 25AxP10, 25AxP11, 25AxP12, 25AxP13, 25AxP14, 25AxP15, 25AxP16, 26AxP4, 26AxP5, 26AxP6, 26AxP7, 26AxP8, 26AxP9, 26AxP10, 26AxP11, 26AxP12, 26AxP13, 26AxP14, 26AxP15, 26AxP16, 27AxP4, 27AxP5, 27AxP6, 27AxP7, 27AxP8, 27AxP9, 27AxP10, 27AxP11, 27AxP12, 27AxP13, 27AxP14, 27AxP15, 27AxP16, 28AxP4, 28AxP5, 28AxP6, 28AxP7, 28AxP8, 28AxP9, 28AxP10, 28AxP11, 28AxP12, 28AxP13, 28AxP14, 28AxP15, 28AxP16, wherein “x” in “nAxPm” refers to the number of Pm molecules modified on one amine molecule.

In some aspects, a synthetic ionizable phospholipid provided herein having a nAxPm formula (IV) may include: 7A1P4, 7A1P5, 7A1P6, 7A1P7, 7A1P8, 7A1P9, 7A1P10, 7A1P11, 7A1P12, 7A1P13, 7A1P14, 7A1P15, 7A1P16, 8A1P4, 8A1P5, 8A1P6, 8A1P7, 8A1P8, 8A1P9, 8A1P10, 8A1P11, 8A1P12, 8A1P13, 8A1P14, 8A1P15, 8A1P16, 9A1P4, 9A1P5, 9A1P6, 9A1P7, 9A1P8, 9A1P9, 9A1P10, 9A1P11, 9A1P12, 9A1P13, 9A1P14, 9A1P15, 9A1P16, 10A1P4, 10A1P5, 10A1P6, 10A1P7, 10A1P8, 10A1P9, 10A1P10, 10A1P11, 10A1P12, 10A1P13, 10A1P14, 10A1P15, 10A1P16, 11A1P4, 11A1P5, 11A1P6, 11A1P7, 11A1P8, 11A1P9, 11A1P10, 11A1P11, 11A1P12, 11A1P13, 11A1P14, 11A1P15, 11A1P16, 12A1P4, 12A1P5, 12A1P6, 12A1P7, 12A1P8, 12A1P9, 12A1P10, 12A1P11, 12A1P12, 12A1P13, 12A1P14, 12A1P15, 12A1P16, 13A1P4, 13A1P5, 13A1P6, 13A1P7, 13A1P8, 13A1P9, 13A1P10, 13A1P11, 13A1P12, 13A1P13, 13A1P14, 13A1P15, 13A1P16, or any combination thereof. In some aspects, a synthetic ionizable phospholipid provided herein having a nAxPm formula (IV) may include: 9A1P9, 9A1P15, 10A1P10, 10A1P16 or any combination thereof.

In some embodiments, synthetic ionizable phospholipids herein are pH-switchable. As used herein, “pH-switchable” refers to a lipid whose conformation changes upon protonation at a defined range of pH values. In some aspects, synthetic ionizable phospholipids herein are pH-switchable at a cytoplasmic pH (e.g., about 7.0 to about 7.5). In some aspects, synthetic ionizable phospholipids herein are pH-switchable at an endosomal and/or lysosomal lumen pH (e.g., about 6.5 to about 4.5). In some embodiments, synthetic ionizable phospholipids herein are pH-switchable at a pH ranging from about 4.0 to about 8.0 (e.g., about 4.0, 4.5, 5.0, 5.5, 6.0, 6.5, 7.0, 7.5, 8.0). In some embodiments, synthetic ionizable phospholipids herein are irreversible (i.e., are not pH-switchable).

In some embodiments, synthetic ionizable phospholipids herein may comprise at least one phosphate group and at least one zwitterion. A zwitterion, also known as inner salt or dipolar ion, is an overall neutral species in which two or more atoms bear opposite formal charges. In some embodiments, synthetic ionizable phospholipids herein may have at least one zwitterion. In some embodiments, synthetic ionizable phospholipids herein may have multiple zwitterions (e.g., more than one zwitterion). In some embodiments, synthetic ionizable phospholipids herein may have about 1 to about 10 (e.g., about 1, 2, 3, 4, 5, 6, 7, 8, 9, 10) zwitterions. In some embodiments, synthetic ionizable phospholipids herein may have at least one irreversible zwitterion. In some embodiments, synthetic ionizable phospholipids herein may have at least one pH-switchable zwitterion. In some embodiments, synthetic ionizable phospholipids herein may have multiple pH-switchable zwitterions (e.g., more than one pH-switchable zwitterion). In some embodiments, synthetic ionizable phospholipids herein may have about 1 to about 10 (e.g., about 1, 2, 3, 4, 5, 6, 7, 8, 9, 10) pH-switchable zwitterions.

In some embodiments, synthetic ionizable phospholipids herein may comprise at least one phosphate group, at least one zwitterion, and at least one hydrophobic domain. In some embodiments, synthetic ionizable phospholipids herein may have at least one tail. In some embodiments, synthetic ionizable phospholipids herein may be multi-tailed (i.e., may have more than one tail). In some embodiments, disclosed multi-tailed ionizable phospholipids may have endosomal membrane destabilization.

In some embodiments, synthetic ionizable phospholipids herein may have at least one hydrophobic tail. In some embodiments, synthetic ionizable phospholipids herein may have more than one hydrophobic tail. In some embodiments, synthetic ionizable phospholipids herein may have about 1 to about 10 (e.g., about 1, 2, 3, 4, 5, 6, 7, 8, 9, 10) hydrophobic tails. In some embodiments, the present disclosure provides pH-switchable, multi-tailed ionizable phospholipids.

In some embodiments, synthetic ionizable phospholipids herein may have at least one hydrophobic tail wherein the hydrophobic tail is an alkyl tail. In some embodiments, synthetic ionizable phospholipids herein may have more than one alkyl tails. In some embodiments, synthetic ionizable phospholipids herein may have about 1 to about 10 (e.g., about 1, 2, 3, 4, 5, 6, 7, 8, 9, 10) alkyl tails. In some embodiments, synthetic ionizable phospholipids herein may have at least one alkyl tail comprising an alkyl chain length of about 5 carbons to about 20 (e.g., about 5, 6, 7, 8, 9, 10, 11, 12, 13, 14, 15, 16, 17, 18, 19, 20) carbons. In some aspects, synthetic ionizable phospholipids herein may have at least one alkyl tail comprising an alkyl chain length of about 8 carbons to about 16 carbons. In some aspects, synthetic ionizable phospholipids herein may have at least one alkyl tail comprising an alkyl chain length of about 8 carbons to about 10 carbons. In some aspects, synthetic ionizable phospholipids herein may have at least one alkyl tail comprising an alkyl chain length of about 9 carbons to about 12 carbons. In some aspects, synthetic ionizable phospholipids herein may have at least one alkyl tail comprising an alkyl chain length of about 13 carbons to about 16 carbons. In some embodiments, synthetic ionizable phospholipids herein may have multiple alkyl tails wherein the length of all the alkyl tails is the same. In some embodiments, synthetic ionizable phospholipids herein may have multiple alkyl tails wherein the length of all the alkyl tails is different from each other.

In some embodiments, synthetic ionizable phospholipids herein may comprise at least one phosphate group, at least one zwitterion, at least one hydrophobic domain, and at least one tertiary amine. As used herein, a tertiary amine refers to an amine in which the nitrogen atom is directly bonded to three carbons of any hybridization which cannot be carbonyl group carbons. In some embodiments, in synthetic ionizable phospholipids herein comprising at least one tertiary amine, the tertiary amine will not be protonated at physiological pH (e.g., about 7.0 to about 7.5). In some aspects, lack of protonation of the tertiary amine in synthetic ionizable phospholipids herein can cause the synthetic ionizable phospholipid to be negatively charged. In some aspects, lack of protonation of the tertiary amine in synthetic ionizable phospholipids herein can cause the synthetic ionizable phospholipid to have difficulty fusing into a cell membrane. In some embodiments, in synthetic ionizable phospholipids herein comprising at least one tertiary amine, the tertiary amine may be protonated at endosomal pH (e.g., about 6.5 to about 4.5). In some aspects, protonation of the tertiary amine in synthetic ionizable phospholipids herein may form at least one zwitterionic head.

In some embodiments, synthetic ionizable phospholipids herein may comprise one tertiary amine, one phosphate group, and three hydrophobic tails. In some embodiments, synthetic ionizable phospholipids herein may comprise one tertiary amine, one phosphate group, and three hydrophobic tails wherein the hydrophobic tails may comprise alkyl chain lengths of about 10 carbons to about 12 carbons.

The present disclosure provides methods of making the synthetic ionizable phospholipids disclosed herein. One of skill in the art will appreciate that standard techniques known in chemical synthesis are suitable for use herein. In some embodiments, methods of making synthetic ionizable phospholipids herein may comprise synthesis through orthogonal reactions by amines (e.g., 1A-28A) and alkylated dioxaphospholane oxide molecules (e.g., P4-P16). In some embodiments, in methods of making synthetic ionizable phospholipids herein, each alkylated dioxaphospholane oxide molecule (e.g., P4-P16) may introduce at least one phosphate group and at least one hydrophobic alkyl chain into the synthetic ionizable phospholipid. In some embodiments, in methods of making synthetic ionizable phospholipids herein, primary, secondary, and/or tertiary amines may consume about one equivalent of the alkylated dioxaphospholane oxide molecule herein (e.g., P4-P16). In some aspects, amines herein having a single primary, secondary or tertiary amine (e.g., 1A-18A) may be reacted with about 1.1 equivalents of an alkylated dioxaphospholane oxide molecule (e.g., P4-P16) to obtain synthetic ionizable phospholipids having nA1Pm according to Formula IV herein. In some aspects, amines herein having multiple amine groups (e.g., 19A-28A) may be reacted with about 2.2 equivalents of an alkylated dioxaphospholane oxide molecule (e.g., P4-P16) to obtain synthetic ionizable phospholipids having nA2Pm according to Formula IV herein. In some aspects, amines herein having multiple amine groups (e.g., 19A-28A) may be reacted with about 3.3 equivalents of an alkylated dioxaphospholane oxide molecule (e.g., P4-P16) to obtain synthetic ionizable phospholipids having nA3Pm according to Formula IV herein. In some aspects, amines herein having multiple amine groups (e.g., 19A-28A) may be reacted with about 4.4 equivalents of an alkylated dioxaphospholane oxide molecule (e.g., P4-P16) to obtain synthetic ionizable phospholipids having nA4Pm according to Formula IV herein. In some aspects, amines herein having multiple amine groups (e.g., 19A-28A) may be reacted with about 5.5 equivalents of an alkylated dioxaphospholane oxide molecule (e.g., P4-P16) to obtain synthetic ionizable phospholipids having nA5Pm according to Formula IV herein.

In some embodiments, methods of making synthetic ionizable phospholipids herein may be conducted in a highly polar organic solvent. Non-limiting examples of highly polar organic solvents for use herein can include water (H₂O), methanol (CH₃OH), dimethyl sulfoxide (DMSO; C₂H₆OS), dimethylformamide (C₃H₇NO), acetonitrile (C₂H₃N), and the like. In some embodiments, methods of making synthetic ionizable phospholipids herein may be conducted in DMSO.

In some embodiments, methods of making synthetic ionizable phospholipids herein may comprise about 0.1 g mL-1 to about 1.0 g mL⁻¹ (e.g., about 0.1, 0.2, 0.3, 0.4, 0.5, 0.6, 0.7, 0.8, 0.9, 1.0 g mL⁻¹) starting material (e.g., amines (e.g., 1A-28A) and alkylated dioxaphospholane oxide molecules (e.g., P4-P16)). In some aspects, methods of making synthetic ionizable phospholipids herein may comprise about 0.3 g mL⁻¹ starting material (e.g., amines (e.g., 1A-28A) and alkylated dioxaphospholane oxide molecules (e.g., P4-P16)).

In some embodiments, methods of making synthetic ionizable phospholipids herein may comprise stirring starting material (e.g., amines (e.g., 1A-28A) and alkylated dioxaphospholane oxide molecules (e.g., P4-P16)) in a highly polar organic solvent for about 1 to about 5 days (e.g., about 1, 2, 3, 4, 5 days). In some aspects, methods of making synthetic ionizable phospholipids herein may comprise stirring starting material (e.g., amines (e.g., 1A-28A) and alkylated dioxaphospholane oxide molecules (e.g., P4-P16)) in a highly polar organic solvent for about 3 days.

In some embodiments, methods of making synthetic ionizable phospholipids herein may comprise stirring starting material (e.g., amines (e.g., 1A-28A) and alkylated dioxaphospholane oxide molecules (e.g., P4-P16)) in a highly polar organic solvent at about 60° C. to about 80° C. (e.g., about 60, 65, 70, 75, 80° C.). In some aspects, methods of making synthetic ionizable phospholipids herein may comprise stirring starting material (e.g., amines (e.g., 1A-28A) and alkylated dioxaphospholane oxide molecules (e.g., P4-P16)) in a highly polar organic solvent at about 70° C.

In some embodiments, methods of making synthetic ionizable phospholipids herein may comprise purification. Non-limiting examples of purification methods suitable for use herein can include column chromatography, vacuum drying, lyophilization, column fractionation, and the like.

### II. Nanoparticles and Lipid Nanoparticles

The present disclosure provides a new class of synthetic ionizable phospholipids for use in nanoparticles and/or lipid nanoparticles (LNPs). The term “nanoparticle” refers to a structure comprising a lipophilic core surrounded by a hydrophilic phase encapsulating the core. In some embodiments, synthetic ionizable phospholipids herein may be used to form one or more nanoparticles. The ionic interaction resulting from the different lipophilic and hydrophilic components of nanoparticle herein may generate independent and/or observable physical characteristics. In some embodiments, nanoparticles herein may have a mean size equal to or less than about 1.0 pm (e.g., about 1000 nm, 750 nm, 500 nm, 250 nm, 150 nm 100 nm, 75 nm, 50 nm, 25 nm, 10 nm, 7.5 nm, 5 nm, 2.5 nm, 1.0 nm). “Mean size” is understood as the average diameter of the population of nanoparticles comprising the lipophilic phase and the hydrophilic phase. The mean size of nanoparticles herein can be measured by standard methods known by the person skilled in the art, and which are described, for example, in the experimental part below. In some embodiments, nanoparticles herein may have a mean particle size equal to or less than 1.0 pm, or between 1.0 nm and 1000 nm, or between 100 nm and 350 nm. One of skill in the art can appreciate that the mean size of the nanoparticles can be influenced by the amount of the lipid component (e.g., at greater amounts the resulting size is equal or greater), by the amount of surfactants (e.g., at greater amounts or higher molecular weight the size is equal or smaller), and/or by the parameters of the preparation method such as, but not limited to, the speed and type of stirring, the temperature of both phases, the duration of the mixing phase, or the like.

In some embodiments, nanoparticles herein may have can have a surface charge the magnitude of which can vary from about −50 mV to about +80 mV. The surface charge of nanoparticles herein can be measured by standard methods known by the person skilled in the art. In some aspects, the surface charge of nanoparticles herein may be measured by means of Z potential.

In some embodiments, a nanoparticle herein may penetrate into one or more cells when the nanoparticle herein is in contact with the one or more cells. In some embodiments, a nanoparticle herein may administer one or more biologically active molecules, small molecules, and/or gene editing therapies to one or more cells when the nanoparticle herein is in contact with the one or more cells. In some embodiments, a nanoparticle herein may penetrate into one or more tissues when the nanoparticle herein is in contact with the one or more tissues. In some embodiments, a nanoparticle herein may administer one or more biologically active molecules, small molecules, and/or gene editing therapies to one or more tissues when the nanoparticle herein is in contact with the one or more tissues. In some embodiments, a nanoparticle herein may penetrate into one or more organs when the nanoparticle herein is in contact with the one or more organs. In some embodiments, a nanoparticle herein may administer one or more biologically active molecules, small molecules, and/or gene editing therapies to one or more cells when the nanoparticle herein is in contact with the one or more organs. In some embodiments, a nanoparticle herein may penetrate into a specific cell type, tissue type, organ, or any combination thereof. In some aspects, a nanoparticle herein may penetrate into a skin cell, a lung cell, a liver cell, a spleen cell, or any combination thereof. In some aspects, a nanoparticle herein may penetrate into a skin tissue, a lung tissue, a liver tissue, a spleen tissue, or any combination thereof. In some aspects, a nanoparticle herein may penetrate into skin, lung, liver, spleen, or any combination thereof.

In some embodiments, synthetic ionizable phospholipids herein may be used to form one or more LNPs. LNPs are spherical vesicles made of ionizable lipids, which can be positively charged at low pH (enabling RNA complexation) and neutral at physiological pH (reducing potential toxic effects, as compared with positively charged lipids, such as liposomes). LNPs may be taken up by cells via endocytosis, and the ionizability of the lipids at low pH (likely) can enable endosomal escape, which allows release of the cargo into the cytoplasm.

In some embodiments, any of the synthetic ionizable phospholipids disclosed herein may be used to form a LNP. In some embodiments, LNPs herein may include one or more multi-tailed ionizable phospholipids. In some embodiments, each of the one or more multi-tailed ionizable phospholipids may include one tertiary amine, one phosphate group, and more than one hydrophobic tail. In some aspects, each of the one or more multi-tailed ionizable phospholipids may include at least one tertiary amine, at least one phosphate group, and more than one hydrophobic tail wherein the hydrophobic tails may comprise alkyl chain lengths of about 5 carbons to about 20 (e.g., about 5, 6, 7, 8, 9, 10, 11, 12, 13, 14, 15, 16, 17, 18, 19, 20) carbons. In some aspects, each of the one or more multi-tailed ionizable phospholipids may include at least one tertiary amine, at least one phosphate group, and more than one hydrophobic tail wherein the hydrophobic tails may comprise alkyl chain lengths of about 10 carbons to about 12 carbons. In some aspects, each of the one or more multi-tailed ionizable phospholipids may include at least one tertiary amine, at least one phosphate group, and three hydrophobic tails wherein the hydrophobic tails may comprise alkyl chain lengths of about 10 carbons to about 12 carbons.

In some embodiments, LNPs herein may include one or more synthetic ionizable phospholipids provided herein having a nAxPm formula (IV) selected from: 1AxP4, 1AxP5, 1AxP6, 1AxP7, 1AxP8, 1AxP9, 1AxP10, 1AxP11, 1AxP12, 1AxP13, 1AxP14, 1AxP15, 1AxP16, 2AxP4, 2AxP5, 2AxP6, 2AxP7, 2AxP8, 2AxP9, 2AxP10, 2AxP11, 2AxP12, 2AxP13, 2AxP14, 2AxP15, 2AxP16, 3AxP4, 3AxP5, 3AxP6, 3AxP7, 3AxP8, 3AxP9, 3AxP10, 3AxP11, 3AxP12, 3AxP13, 3AxP14, 3AxP15, 3AxP16, 4AxP4, 4AxP5, 4AxP6, 4AxP7, 4AxP8, 4AxP9, 4AxP10, 4AxP11, 4AxP12, 4AxP13, 4AxP14, 4AxP15, 4AxP16, 5AxP4, 5AxP5, 5AxP6, 5AxP7, 5AxP8, 5AxP9, 5AxP10, 5AxP11, 5AxP12, 5AxP13, 5AxP14, 5AxP15, 5AxP16, 6AxP4, 6AxP5, 6AxP6, 6AxP7, 6AxP8, 6AxP9, 6AxP10, 6AxP11, 6AxP12, 6AxP13, 6AxP14, 6AxP15, 6AxP16, 7AxP4, 7AxP5, 7AxP6, 7AxP7, 7AxP8, 7AxP9, 7AxP10, 7AxP11, 7AxP12, 7AxP13, 7AxP14, 7AxP15, 7AxP16, 8AxP4, 8AxP5, 8AxP6, 8AxP7, 8AxP8, 8AxP9, 8AxP10, 8AxP11, 8AxP12, 8AxP13, 8AxP14, 8AxP15, 8AxP16, 9AxP4, 9AxP5, 9AxP6, 9AxP7, 9AxP8, 9AxP9, 9AxP10, 9AxP11, 9AxP12, 9AxP13, 9AxP14, 9AxP15, 9AxP16, 10AxP4, 10AxP5, 10AxP6, 10AxP7, 10AxP8, 10AxP9, 10AxP10, 10AxP11, 10AxP12, 10AxP13, 10AxP14, 10AxP15, 10AxP16, 11AxP4, 11AxP5, 11AxP6, 11AxP7, 11AxP8, 11AxP9, 11AxP10, 11AxP11, 11AxP12, 11AxP13, 11AxP14, 11AxP15, 11AxP16, 12AxP4, 12AxP5, 12AxP6, 12AxP7, 12AxP8, 12AxP9, 12AxP10, 12AxP11, 12AxP12, 12AxP13, 12AxP14, 12AxP15, 12AxP16, 13AxP4, 13AxP5, 13AxP6, 13AxP7, 13AxP8, 13AxP9, 13AxP10, 13AxP11, 13AxP12, 13AxP13, 13AxP14, 13AxP15, 13AxP16, 14AxP4, 14AxP5, 14AxP6, 14AxP7, 14AxP8, 14AxP9, 14AxP10, 14AxP11, 14AxP12, 14AxP13, 14AxP14, 14AxP15, 14AxP16, 15AxP4, 15AxP5, 15AxP6, 15AxP7, 15AxP8, 15AxP9, 15AxP10, 15AxP11, 15AxP12, 15AxP13, 15AxP14, 15AxP15, 15AxP16, 16AxP4, 16AxP5, 16AxP6, 16AxP7, 16AxP8, 16AxP9, 16AxP10, 16AxP11, 16AxP12, 16AxP13, 16AxP14, 16AxP15, 16AxP16, 17AxP4, 17AxP5, 17AxP6, 17AxP7, 17AxP8, 17AxP9, 17AxP10, 17AxP11, 17AxP12, 17AxP13, 17AxP14, 17AxP15, 17AxP16, 18AxP4, 18AxP5, 18AxP6, 18AxP7, 18AxP8, 18AxP9, 18AxP10, 18AxP11, 18AxP12, 18AxP13, 18AxP14, 18AxP15, 18AxP16, 19AxP4, 19AxP5, 19AxP6, 19AxP7, 19AxP8, 19AxP9, 19AxP10, 19AxP11, 19AxP12, 19AxP13, 19AxP14, 19AxP15, 19AxP16, 20AxP4, 20AxP5, 20AxP6, 20AxP7, 20AxP8, 20AxP9, 20AxP10, 20AxP11, 20AxP12, 20AxP13, 20AxP14, 20AxP15, 20AxP16, 21AxP4, 21AxP5, 21AxP6, 21AxP7, 21AxP8, 21AxP9, 21AxP10, 21AxP11, 21AxP12, 21AxP13, 21AxP14, 21AxP15, 21AxP16, 22AxP4, 22AxP5, 22AxP6, 22AxP7, 22AxP8, 22AxP9, 22AxP10, 22AxP11, 22AxP12, 22AxP13, 22AxP14, 22AxP15, 22AxP16, 23AxP4, 23AxP5, 23AxP6, 23AxP7, 23AxP8, 23AxP9, 23AxP10, 23AxP11, 23AxP12, 23AxP13, 23AxP14, 23AxP15, 23AxP16, 24AxP4, 24AxP5, 24AxP6, 24AxP7, 24AxP8, 24AxP9, 24AxP10, 24AxP11, 24AxP12, 24AxP13, 24AxP14, 24AxP15, 24AxP16, 25AxP4, 25AxP5, 25AxP6, 25AxP7, 25AxP8, 25AxP9, 25AxP10, 25AxP11, 25AxP12, 25AxP13, 25AxP14, 25AxP15, 25AxP16, 26AxP4, 26AxP5, 26AxP6, 26AxP7, 26AxP8, 26AxP9, 26AxP10, 26AxP11, 26AxP12, 26AxP13, 26AxP14, 26AxP15, 26AxP16, 27AxP4, 27AxP5, 27AxP6, 27AxP7, 27AxP8, 27AxP9, 27AxP10, 27AxP11, 27AxP12, 27AxP13, 27AxP14, 27AxP15, 27AxP16, 28AxP4, 28AxP5, 28AxP6, 28AxP7, 28AxP8, 28AxP9, 28AxP10, 28AxP11, 28AxP12, 28AxP13, 28AxP14, 28AxP15, 28AxP16, wherein “x” in “nAxPm” refers to the number of Pm molecules modified on one amine molecule.

In some embodiments, LNPs herein may include one or more synthetic ionizable phospholipids provided herein having a nAxPm formula (IV) selected from: 7A1P4, 7A1P5, 7A1P6, 7A1P7, 7A1P8, 7A1P9, 7A1P10, 7A1P11, 7A1P12, 7A1P13, 7A1P14, 7A1P15, 7A1P16, 8A1P4, 8A1P5, 8A1P6, 8A1P7, 8A1P8, 8A1P9, 8A1P10, 8A1P11, 8A1P12, 8A1P13, 8A1P14, 8A1P15, 8A1P16, 9A1P4, 9A1P5, 9A1P6, 9A1P7, 9A1P8, 9A1P9, 9A1P10, 9A1P11, 9A1P12, 9A1P13, 9A1P14, 9A1P15, 9A1P16, 10A1P4, 10A1P5, 10A1P6, 10A1P7, 10A1P8, 10A1P9, 10A1P10, 10A1P11, 10A1P12, 10A1P13, 10A1P14, 10A1P15, 10A1P16, 11A1P4, 11A1P5, 11A1P6, 11A1P7, 11A1P8, 11A1P9, 11A1P10, 11A1P11, 11A1P12, 11A1P13, 11A1P14, 11A1P15, 11A1P16, 12A1P4, 12A1P5, 12A1P6, 12A1P7, 12A1P8, 12A1P9, 12A1P10, 12A1P11, 12A1P12, 12A1P13, 12A1P14, 12A1P15, 12A1P16, 13A1P4, 13A1P5, 13A1P6, 13A1P7, 13A1P8, 13A1P9, 13A1P10, 13A1P11, 13A1P12, 13A1P13, 13A1P14, 13A1P15, 13A1P16, or any combination thereof. In some aspects, LNPs herein may include one or more synthetic ionizable phospholipids provided herein having a nAxPm formula (IV) selected from: 9A1P9, 9A1P15, 10A1P10, 10A1P16 or any combination thereof.

LNPs usually contain a helper lipid to promote cell binding, cholesterol to fill the gaps between the lipids, and a polyethylene glycol (PEG) to reduce opsonization by serum proteins and reticuloendothelial clearance.

In some embodiments, LNPs herein may include one or more synthetic ionizable phospholipids provided herein and at least one helper lipid. In some embodiments, LNPs herein may include one or more synthetic ionizable phospholipids provided herein and at least one helper lipid selected from: 1,2-dioleoyl-sn-glycero-3-phosphoethanolamine (DOPE), N-methyldioctadecylamine (MDOA), 1,2-dioleoyl-3-dimethylammonium-propane (DODAP), dimethyldioctadecylammonium bromide salt (DDAB), 1,2-dioleoyl-3-trimethylammonium-propane (DOTAP), and any combination thereof. In some embodiments, LNPs herein may include one or more synthetic ionizable phospholipids provided herein and at least one zwitterionic helper lipid (e.g., DOPE), ionizable cationic helper lipid (e.g., MDOA, DODAP), permanently cationic helper lipid (e.g., DDAB, DOTAP), or any combination thereof.

In some embodiments, LNPs herein may include one or more synthetic ionizable phospholipids provided herein and at least one cholesterol and/or a cholesterol derivative. As used herein, “cholesterol derivative” refers to any compound consisting essentially of a cholesterol structure, including additions, substitutions and/or deletions thereof. The term cholesterol derivative herein can also include steroid hormones and bile acids as are generally recognized in the art. Non-limiting examples of cholesterol derivatives suitable for use herein can include dihydrocholesterol, ent-cholesterol, epi-cholesterol, desmosterol, cholestanol, cholestanone, cholestenone, sitosterol, cholesteryl-2′-hydroxyethyl ether, cholesteryl-4′-hydroxybutyl ether, 3β-[N-(N′N′-dimethylaminoethyl)carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt or ester thereof.

In some embodiments, LNPs herein may include one or more synthetic ionizable phospholipids provided herein and at least one PEG or PEG-modified lipids. As used herein, a PEG-modified lipid, or “PEG lipid” refers to a lipid modified with polyethylene glycol (PEG). Such species may be alternately referred to as PEGylated lipids. Non-limiting examples of PEG-modified lipids suitable for use herein can include PEG-modified phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, but not limited to, a PEG-modified lipid for use herein may be PEG-c-DOMG (R-3-[(ω-methoxypoly(ethyleneglycol)2000)carbamoyl]-1,2-dimyristyloxy-propyl-3-amine poly(ethylene glycol)); PEG-DMG (1,2-dimyristoyl-rac-glycero-3-methoxypolyethylene glycol poly(ethylene glycol)); PEG-DLPE (1,2-Dilauroyl-sn-glycero-3-phosphorylglycerol sodium salt-poly(ethylene glycol)); PEG-DM PE (dimethyl-2-(dimethylphosphino)ethylphosphine-poly(ethylene glycol)); PEG-DPPC (1,2-dipalmitoyl-sn-glycero-3-phosphocholine-poly(ethylene glycol)); PEG-DSPE (1,2-distearoyl-sn-glycero-3-phosphoethanolamine-poly(ethylene glycol)); PEG-DPPE (1,2-dipalmitoyl-sn-glycero-3-phosphoethanolamine-N-[monomethoxy poly(ethylene glycol)), and the like.

In some embodiments, a PEG-modified lipid for use herein may comprise a PEG moiety having a size of from about 1000 daltons to about 20,000 daltons. In some aspects, a PEG-modified lipid for use herein may comprise a PEG moiety having a size of about 1000 daltons, about 2000 daltons, about 5000 daltons, about 10,000 daltons, about 15,000 daltons, or about 20,000 daltons. In some aspects, LNPs herein may include one or more synthetic ionizable phospholipids provided herein and at least one PEG or PEG-modified lipids wherein the PEG moiety may have a size of about 2000 daltons. Examples of useful PEG-lipids for use in making the LNPs described herein include, but are not limited to, 1,2-Diacyl-sn-Glycero-3-Phosphoethanolamine-N-[Methoxy(Polyethylene glycol)-350] (mPEG 350 PE); 1,2-Diacyl-sn-Glycero-3-Phosphoethanolamine-N-[Methoxy(Polyethylene glycol)-550] (mPEG 550 PE); 1,2-Diacyl-sn-Glycero-3-Phosphoethanolamine-N-[Methoxy(Polyethylene glycol)-750] (mPEG 750 PE); 1,2-Diacyl-sn-Glycero-3-Phosphoethanolamine-N-[Methoxy(Polyethylene glycol)-1000] (mPEG 1000 PE); 1,2-Diacyl-sn-Glycero-3-Phosphoethanolamine-N-[Methoxy(Polyethylene glycol)-2000] (mPEG 2000 PE); 1,2-Diacyl-sn-Glycero-3-Phosphoethanolamine-N-[Methoxy(Polyethylene glycol)-3000] (mPEG 3000 PE); 1,2-Diacyl-sn-Glycero-3-Phosphoethanolamine-N-[Methoxy(Polyethylene glycol)-5000] (m PEG 5000 PE); N-Acyl-Sphingosine-1-[Succinyl(Methoxy Polyethylene Glycol) 750] (m PEG 750 Ceramide); N-Acyl-Sphingosine-1-[Succinyl(Methoxy Polyethylene Glycol) 2000] (mPEG 2000 Ceramide); and N-Acyl-Sphingosine-1-[Succinyl(Methoxy Polyethylene Glycol) 5000] (m PEG 5000 Ceramide). In some aspects, LNPs herein may include one or more synthetic ionizable phospholipids provided herein and 1,2-dimyristoyl-rac-glycero-3-methoxy(poly(ethylene glycol-2000)) (DMG-PEG2000).

The relative amounts of ionizable lipid, helper lipid, cholesterol and PEG that make up LNPs may substantially affect the efficacy of lipid nanoparticles. In some embodiments, LNPs herein may include one or more synthetic ionizable phospholipids provided herein, at least one helper lipid, and at least one cholesterol and/or cholesterol derivative in a 55:30:45, 25:30:30, or 60:30:40 molar ratio. One of skill in the art will appreciate that the molar ratio of one or more synthetic ionizable phospholipids provided herein, at least one helper lipid, and at least one cholesterol and/or cholesterol derivative can be optimized as needed, particularly for a given application and/or administration route of the LNPs herein. In some aspects, LNPs herein may include one or more synthetic ionizable phospholipids provided herein, 1,2-dioleoyl-sn-glycero-3-phosphoethanolamine (DOPE), and cholesterol in a 55:30:45 molar ratio. In some aspects, LNPs herein may include one or more synthetic ionizable phospholipids provided herein, N-methyldioctadecylamine (MDOA), and cholesterol in a 25:30:30 molar ratio. In some aspects, LNPs herein may include one or more synthetic ionizable phospholipids provided herein, 1,2-dioleoyl-3-dimethylammonium-propane (DODAP), and cholesterol in a 25:30:30 molar ratio. In some aspects, LNPs herein may include one or more synthetic ionizable phospholipids provided herein, 5A2-SC8, and cholesterol in a 25:30:30 molar ratio. In some aspects, LNPs herein may include one or more synthetic ionizable phospholipids provided herein, dimethyldioctadecylammonium bromide salt (DDAB), and cholesterol in a 60:30:40 molar ratio. In some aspects, LNPs herein may include one or more synthetic ionizable phospholipids provided herein, 1,2-dioleoyl-3-trimethylammonium-propane (DOTAP), and cholesterol in a 60:30:40 molar ratio.

In some embodiments, LNPs herein may include one or more synthetic ionizable phospholipids provided herein to target one or more cell types. In some embodiments, LNPs comprising synthetic ionizable phospholipids provided herein can make the LNPs selective for one or more cell types. In some embodiments, LNPs comprising synthetic ionizable phospholipids provided herein can make LNPs unload cargo at one or more selective cell types. In some embodiments, LNPs herein may selectively target one or more cell types. In some embodiments, LNPs herein may selectively target skin cells, spleen cells, liver cells, lung cells or a combination thereof. In some embodiments, LNPs herein may selectively target one or more tissue types. In some embodiments, LNPs herein may selectively target skin, spleen, liver, lung or a combination thereof. In some aspects, LNPs herein comprising the synthetic ionizable phospholipid 9A1P9 and the helper lipid 5A2-SC8 may selectively target the liver. In some aspects, LNPs herein comprising the synthetic ionizable phospholipid 9A1P9 and the helper lipid DDAB may selectively target the lung. In some aspects, LNPs herein comprising the synthetic ionizable phospholipid 10A1P16 and the helper lipid MDOA may selectively target the spleen.

LNP size can impact the behavior of lipid nanoparticles in vivo. In some descriptions, e.g., where diameter is a relevant measurement, such as in spherical and other shaped vesicles having a measurable diameter, the terms “size” and “diameter” are used interchangeably. The size of LNPs disclosed herein may be determined by Dynamic Light Scattering (DLS) and/or nanoparticle tracking analysis (NTA).

In some embodiments, LNPs herein may be about 20 nm to about 1000 nm in diameter or size. In some embodiments, LNPs herein may be about 20 nm to about 200 nm in size. In some embodiments, LNPs herein may about 20 nm to about 190 nm or about 25 nm to about 190 nm in size. In some embodiments, LNPs herein may be about 30 nm to about 180 nm in size. In some embodiments, LNPs herein may be about 35 nm to about 170 nm in size. In some embodiments, LNPs herein may be about 40 nm to about 160 nm in size. In some embodiments, LNPs herein may be about 50 nm to about 150 nm, about 60 nm to about 140 nm, about 70 nm to about 130 nm, about 80 nm to about 120 nm, or about 90 nm to about 110 nm in size. In some embodiments, LNPs herein may be about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm in size or diameter.

In some embodiments, an average LNP size in a LNP composition or plurality of LNPs may be about 20 nm to about 1000 nm (e.g., about 20, 40, 60, 80, 100, 150, 200, 250, 300, 350, 400, 450, 500, 550, 600, 650, 700, 750, 800, 850, 900, 950, 1000) in diameter in average size. In some embodiments, LNP size in a LNP composition or plurality of LNPs may be homogenous at about 20 nm to about 1000 nm (e.g., about 20, 40, 60, 80, 100, 150, 200, 250, 300, 350, 400, 450, 500, 550, 600, 650, 700, 750, 800, 850, 900, 950, 1000) in diameter in size. In some embodiments, LNP size in a LNP composition or plurality of LNPs may be heterogeneous at about 20 nm to about 1000 nm (e.g., about 20, 40, 60, 80, 100, 150, 200, 250, 300, 350, 400, 450, 500, 550, 600, 650, 700, 750, 800, 850, 900, 950, 1000) in diameter in average size. In some embodiments, LNP size in a LNP composition or plurality of LNPs may be heterogeneous wherein about 50% to about 99% of the LNPs average at about 20 nm to about 1000 nm (e.g., about 20, 40, 60, 80, 100, 150, 200, 250, 300, 350, 400, 450, 500, 550, 600, 650, 700, 750, 800, 850, 900, 950, 1000) in diameter in average size.

In some embodiments, LNPs comprising one or more synthetic ionizable phospholipids herein may have a negative charge. In some embodiments, LNPs comprising one or more synthetic ionizable phospholipids herein may have a negative charge outside of a cell (e.g., in serum). In some embodiments, LNPs comprising one or more synthetic ionizable phospholipids herein may have a negative surface zeta potential. In some embodiments, LNPs comprising one or more synthetic ionizable phospholipids herein may have a negative surface zeta potential ranging from about −20 mV to about −0.5 mV. In some embodiments, LNPs comprising one or more synthetic ionizable phospholipids herein may have a negative surface zeta potential of about −20 mV, about −15 mV, about −10 mV, about −5 mV, about −2.5 mV, about −1.0 mV, or about −0.5 mV.

In some embodiments, LNPs comprising one or more synthetic ionizable phospholipids herein may have no charge (e.g., net neural charge). In some embodiments, LNPs comprising one or more synthetic ionizable phospholipids herein may have no charge inside of a cell (e.g., in the cell lumen). In some embodiments, a LNP comprising one or more synthetic ionizable phospholipids herein may have a negative charge outside of a cell and no charge after the LNP crosses the cell membrane into the cell lumen. In some embodiments, a LNP comprising one or more synthetic ionizable phospholipids herein may have a negative surface zeta potential outside of a cell and no surface charge after the LNP crosses the cell membrane into the cell lumen. In some embodiments, a LNP comprising one or more synthetic ionizable phospholipids herein may have a negative surface zeta potential ranging from about −20 mV to about −0.5 mV outside of a cell and no surface charge (e.g., ˜0 mV) after the LNP crosses the cell membrane into the cell lumen.

In some embodiments, LNPs comprising one or more synthetic ionizable phospholipids herein may have a pKa suitable for in vivo uses. In some embodiments, LNPs comprising one or more synthetic ionizable phospholipids herein may have a pKa ranging from about 5.5 to about 7.5 (e.g., about 5.5, 6.0, 6.5, 7.0, 7.5).

A variety of methods can be used for preparing the LNPs described herein. Such methods are known in the art or disclosed herein, for example, the methods described in Lichtenberg and Barenholz in Methods of Biochemical Analysis, Volume 33, 337-462 (1988). See also Szoka et al., Ann. Rev. Biophys. Bioeng. 9:467 (1980); U.S. Pat. Nos. 4,235,871, 4,501,728, and 4,837,028; Liposomes, Marc J. Ostro, ed., Marcel Dekker, Inc., New York, 1983, Chapter 1; and Hope, et al., Chem. Phys. Lip. 40:89 (1986), the relevant disclosures of each of which are incorporated herein by reference.

Any of the LNPs described herein can be used as vehicles for carrying biological molecules (cargo) to facilitate delivery of the biological molecules to a subject. In addition to other superior features of LNPs disclosed herein, LNPs can protect the cargo loaded therein from degradation, for example, from digestion by enzymes. Thus, also provided herein are cargo-loaded LNPs, which can be used to deliver the loaded cargo to a subject for diagnostic and/or therapeutic purposes. In some aspects, the cargo can be a therapeutic agent. In some aspects, the cargo can be a gene editing agent.

In some embodiments, the present disclosure provides a cargo-loaded LNP or a therapeutic-loaded LNP. The term “cargo-loaded LNP,” “therapeutic-loaded LNP” or “therapeutic agent-loaded LNP” is meant to be inclusive of the loading of one or more cargos, including therapeutic agents, diagnostic agents, and agents for use in gene editing. As used herein, the term “loaded” or “loading” as used in reference to a “cargo-loaded LNP,” “therapeutic-loaded LNP” or “therapeutic agent-loaded LNP” refers to a LNP having one or more cargos (which can be biological molecules such as therapeutic agents, diagnostic agents, agents for use in gene editing) that are either (1) encapsulated inside the LNP; (2) associated with or partially embedded within the lipid membrane of the LNP (i.e. partly protruding inside the interior of the LNP); (3) associated with or bound to the outer portion of the lipid membrane and associated components (i.e., partly protruding or fully outside the LNP); or (4) entirely disposed within the lipid membrane of the LNP (i.e., entirely contained within the lipid membrane).

The term “cargo-loading” refers to the process of loading, adding, or including exogenous cargo or therapeutic to the LNP such that any one or more of the above (1)-(4) resultant cargo loaded or therapeutic-loaded vesicles is accomplished. Thus, in some embodiments, the cargo is encapsulated inside the LNP. In some embodiments, the cargo is associated with or partially embedded within the lipid membrane of the LNP (i.e. partly protruding inside the interior of the vesicle). In some embodiments, the cargo is associated with or bound to the outer portion of the lipid membrane (i.e., partly protruding outside the LNP). In some embodiments, the cargo is entirely disposed within the lipid membrane of the vesicle (i.e., entirely contained within the lipid membrane). As used herein, the term “cargo” is meant to include any biomolecule or agent that can be loaded into or by a LNP, including, for example, a biologic (e.g., a peptide, a protein, an antibody, an aptamer, a nucleic acid, an oilgo), small molecule, therapeutic agent, and/or diagnostic agent.

In some embodiments, one or more cargos may be present on the interior or internal surface of the LNP. In some embodiments, the one or more cargos present on the interior or internal surface of the LNP may be associated with the LNP, e.g., via chemical interaction, electromagnetic interaction, hydrophobic interaction, electrostatic interaction, van der Waals interaction, linkage, bond (hydrogen bond, ionic bond, covalent bond, etc.). In some embodiments, LNPs herein can encapsulate one or more cargos.

In some embodiments, LNPs herein may be loaded with a single cargo, for example, a single therapeutic agent. In some embodiments, LNPs herein may be loaded with two (or more) different cargos. In some embodiments, LNPs herein may be loaded with two or more molecules or copies of a single cargo or two (or more) different cargos. In some embodiments, LNPs herein may be loaded with three or more molecules or copies of a single cargo or two (or more) different cargos. In some embodiments, LNPs herein may be loaded with 2-5 molecules or copies of a single cargo or two (or more) different cargos. In some embodiments, LNPs herein and/or a pharmaceutical composition thereof may be loaded with 1-4,000, 10-4,000, 50-3,500, 100-3,000, 200-2,500, 300-1,500, 500-1,200, 750-1,000, 1-2,000, 1-1,000, 1-500, 10-400, 50-300, 1-250, 1-100, 2-50, 2-25, 2-15, 2-10, 3-50, 3-25, 3-25, 3-10, 4-50, 4-25, 4-15, 4-10, 5-50, 5-25, 5-15, or 5-10 molecules or copies of a single cargo or two (or more) different cargos, or any increment therein.

Methods of loading cargo into LNPs are known in the art can be used to load cargos into the LNPs of the present disclosure. Non-limiting examples of methods of loading cargo into LNPs suitable for use herein include pH gradients, metal ion gradients, transmembrane gradients, surface loading, fusion loading, and the like. The cargo in the cargo-loaded LNPs described herein can be of any type. In some embodiments, the cargo of the LNPs herein may be selected from the group consisting of an active pharmaceutical ingredient, a nucleic acid, ncRNA, siRNA, miRNA, tRNA, mRNA, shRNA, sgRNA, a CRISPR/Cas9 DNA sequence, a CRISPR/Cas12 DNA sequence, a CRISPR/Cas13 sequence, a adenosine deaminases acting on RNA (ADAR) sequence, a zinc finger nuclease (ZFN), transcription activator-like effector nucleases (TALENs), base editor, single stranded DNA (ssDNA), plasmid DNA (pDNA), circular RNA (circRNA), an antisense oligonucleotide (AO), a small molecule drug, protein, and any combination thereof.

In some embodiments, the cargo in the cargo-loaded LNPs herein can be a biological molecule. As used herein, the term “biological molecule” is used interchangeably with the term “biologic therapeutic agent”. In some embodiments, the cargo in the cargo-loaded LNPs herein can be a small molecule. In some embodiments, the cargo in the cargo-loaded LNPs herein can be an active pharmaceutical ingredient.

In some embodiments, LNPs herein may comprise a cargo (e.g., a biological molecule) that is a protein, peptide, aptamer, antibody, antibody fragment, or any combination thereof. In some embodiments, LNPs herein may comprise a cargo (e.g., a biological molecule) that is a nucleic acid. In some aspects, a nucleic acid cargo can be, for example, an oligonucleotide therapeutic agent, such as a single-stranded or double-stranded oligonucleotide therapeutic agent. In some examples, the oligonucleotide therapeutic agent can be a single-stranded or double-stranded DNA, iRNA, shRNA, siRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′O-Me-RNA, 2′-MEO-RNA, 2′-F-RNA), or analog or conjugate thereof. In some embodiments, the cargo in the cargo-loaded LNPs herein can be mRNA.

In some embodiments, LNPs herein may be used for the delivery of a CRISPR-Cas system. The “CRISPR/Cas” system or “CRISPR/Cas-mediated gene editing” refers to a type II CRISPR/Cas system (e.g., CRISPR/Cas9), a type V CRISPR/Cas system (e.g., CRISPR/Cas12), and/or a type VI CRISPR/Cas system (e.g., CRISPR/Cas13), that has been modified for genome editing/engineering. It is typically comprised of a “guide” RNA (gRNA) and a non-specific CRISPR-associated endonuclease (e.g., Cas9, Cas12, Cas13, or any variant thereof). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas-binding and a user-defined “20 nucleotide “spacer” or “targeting” sequence which defines the genomic target to be modified. The genomic target of Cas can be changed by changing the targeting sequence present in the sgRNA. In some embodiments, LNPs herein may comprise a cargo having one or more components of a CRISPR-Cas system. In some embodiments, a cargo having one or more components of a CRISPR-Cas system may include mRNA, sgRNA, a CRISPR/Cas DNA sequence CRISPR/Cas ribonucleoprotein (RNP) complex, and any combination thereof.

### III. Pharmaceutical Compositions

The present disclosure also provides pharmaceutical compositions comprising any of the LNPs described herein, which may encapsulate one or more of the cargos also described herein, and a pharmaceutically acceptable carrier or excipient. The carrier in the pharmaceutical composition must be “acceptable” in the sense that it is compatible with the active ingredient of the composition, and preferably, capable of stabilizing the active ingredient and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover, the disclosures of which are incorporated by reference.

In some embodiments, pharmaceutical compositions herein can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Depending on the route of administration, and form of medication, different carriers and/or excipients may be used herein. Excipients for use herein include but are not limited to antiadherents, binders, coatings disintegrants, fillers, flavors (such as sweeteners) and colors, glidants, lubricants, preservatives, sorbents. Carriers and/or excipients described herein may also include vehicles and/or diluents, wherein: “vehicles” indicates any of various media acting usually as solvents or carriers; “diluent” indicates a diluting agent which is issued to dilute an active ingredient of a composition; suitable diluent include any substance that can decrease the viscosity of a medicinal preparation.

The type and amounts of carriers and/or excipients are chosen in function of the chosen pharmaceutical form; suitable pharmaceutical forms are liquid systems like solutions, infusions, suspensions; semisolid systems like colloids, gels, pastes or cremes; solid systems like powders, granulates, tablets, capsules, pellets, microgranulates, minitablets, microcapsules, micropellets, suppositories; etc. Each of the above systems can be suitably be formulated for normal, delayed or accelerated release, using techniques well-known in the art.

Pharmaceutical compositions comprising the LNPs described herein can be prepared according to standard techniques, as well as those techniques described herein. In some examples, the pharmaceutical compositions are formulated for parenteral administration, including intracanalicular administration, intravenous administration, subcutaneous administration, intracutaneous administration, intraperitoneal administration, intrathecal administration, and intramuscular administration. In some examples, the pharmaceutical compositions herein may be administered intravenously by a bolus injection or infusion. Suitable formulations for use in the present invention are found in Remington's Pharmaceutical Sciences, Mack Publishing Company, Philadelphia, Pa., 17th ed. (1985), the disclosures of which are incorporated by reference.

In some embodiments, pharmaceutical compositions herein can be formulated for injection, such as intravenous infusion. A sterile injectable composition, e.g., a sterile injectable aqueous or oleaginous suspension, can be formulated according to techniques known in the art using suitable dispersing or wetting agents (such as Tween 80) or suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically.

In some embodiments, pharmaceutical compositions described herein can be in unit dosage forms such as tablets, pills, capsules, powders, granules, solutions or suspensions, or suppositories, for oral, parenteral or rectal administration, or administration by inhalation or insufflation. For preparing solid compositions such as tablets, the LNPs disclosed herein can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In some embodiments, pharmaceutical compositions described herein can be emulsions. Suitable emulsions may be prepared using commercially available fat emulsions, such as Intralipid™, Liposyn™, Infonutrol™, Lipofundin™ and Lipiphysan™. LNPs herein may be either added in a pre-mixed emulsion composition or alternatively it may be added in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0. The emulsion compositions can be those prepared by mixing LNPs with Intralipid™ or the components thereof (soybean oil, egg phospholipids, glycerol and water). In some embodiments, pharmaceutical compositions described herein can be emulsions for topical administration (e.g., for treating the skin).

### IV. Methods of Use

In certain embodiments, the present disclosure also provides for methods of introducing one or more cargos (e.g., a nucleic acid molecule, an active pharmaceutical ingredient) to a cell, comprising contacting the cell with a composition disclosed herein. In some embodiments, methods herein can include delivering one or more cargos (e.g., a nucleic acid molecule, an active pharmaceutical ingredient) herein to a cell, comprising contacting the cell or cell layer with LNP disclosed herein. In some embodiments of this method, LNPs herein can deliver one or more heterologous molecules to a cell. In accordance with these embodiments, LNPs herein can deliver one or more therapeutic heterologous molecules to a cell. In some examples, one or more therapeutic heterologous molecules delivered to a cell using the methods herein may be a therapeutic protein, a therapeutic DNA, and/or therapeutic RNA. In some embodiments, the therapeutic protein can be a monoclonal antibody or a fusion protein. In some embodiments, the therapeutic DNA and/or RNA can be an antisense oligonucleotide, siRNA, shRNA, mRNA, a DNA oligonucleotide, and the like. In some aspects, LNPs herein can deliver one or more therapeutic mRNAs to a cell.

In some embodiments, the present disclosure also provides for methods of introducing cargo (e.g., a nucleic acid molecule, an active pharmaceutical ingredient) into a cell, comprising contacting the cell with a LNP and/or a pharmaceutical composition disclosed herein. In some embodiments, methods herein can include delivering cargo (e.g., a nucleic acid molecule, an active pharmaceutical ingredient) to specific cell type. In some embodiments, methods herein can include delivering cargo (e.g., a nucleic acid molecule, an active pharmaceutical ingredient) to specific cell type selected from a liver cell, a lung cell, a spleen cell, and/or a skin cell. In some embodiments, methods herein can include delivering cargo (e.g., a nucleic acid molecule, an active pharmaceutical ingredient) to a liver cell, comprising contacting the liver cell with a LNP disclosed herein. In some embodiments, methods herein can include delivering cargo (e.g., a nucleic acid molecule, an active pharmaceutical ingredient) to a lung cell, comprising contacting the lung cell with a LNP disclosed herein. In some embodiments, methods herein can include delivering cargo (e.g., a nucleic acid molecule, an active pharmaceutical ingredient) to a spleen cell, comprising contacting the spleen cell with a LNP disclosed herein. In some embodiments, methods herein can include delivering cargo (e.g., a nucleic acid molecule, an active pharmaceutical ingredient) to a skin cell, comprising contacting the skin cell with a LNP disclosed herein. In certain embodiments, the present disclosure also provides for methods of introducing cargo (e.g., a nucleic acid molecule, an active pharmaceutical ingredient) to a lung tissue, a liver tissue, a spleen tissue, a skin tissue or any combination thereof, comprising contacting the cell with a LNP and/or composition disclosed herein.

Any of the LNPs and/or pharmaceutical compositions herein can be used for delivering a therapeutic agent, diagnostic agent, or gene editing system to a desired target site. In some embodiments, LNPs and/or pharmaceutical compositions herein can be used for delivering a therapeutic agent, diagnostic agent, or gene editing system to lung, liver, skin, and/or spleen.

In some embodiments, any of the LNPs and/or pharmaceutical compositions herein can be used for delivering a therapeutic agent, diagnostic agent, or gene editing system to treat and/or prevent a diseases, condition, or disorder in a subject. To practice this use, an effective amount of a pharmaceutical composition comprising the LNPs herein can be administered to a subject in need of the treatment (e.g., a mammal subject, a human subject) via a suitable route, such as those described herein. Also to practice this use, an effective amount of a pharmaceutical composition comprising any of the LNPs described herein, which encapsulates a therapeutic agent, diagnostic agent, or gene editing system can be administered to a subject in need of the treatment (e.g., a human subject) via a suitable route, such as those described herein. “An effective amount” as used herein refers to the amount of each active agent required to confer therapeutic effects on the subject, either alone or in combination with one or more other active agents. Effective amounts vary, as recognized by those skilled in the art, depending on route of administration, excipient usage, and co-usage with other active agents. Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, the synthetic ionizable phospholipids, LNPS, pharmaceutical compositions, and methods described herein may be used to treat a lung disease or a lung disorder. Non-limiting examples of lung diseases and/or lung disorders suitable for treatment via the methods herein can include chronic obstructive pulmonary disease (COPD), asthma, acute tracheal bronchitis, pneumonia, tuberculosis, lung cancer, influenza infection, SARS-CoV2 infection, surfactant protein deficiency, cystic fibrosis, Alpha-1 antitrypsin (AAT) deficiency, and the like.

In some embodiments, the synthetic ionizable phospholipids, LNPs, pharmaceutical compositions, and methods described herein may be used to treat a liver disease or a liver disorder. Non-limiting examples of liver diseases and/or liver disorders suitable for treatment via the methods herein can include phenylketonuria (PKU), ornithine transcarbamylase (OTC) deficiency, arginase-1 deficiency, alpha-1 antitrypsin deficiency, tyrosinemia type 1 (HT1), mucopolysaccharidoses, hemophilia, hypercholesterolemia, cirrhosis, liver cancer, nonalcoholic fatty liver disease (NAFLD), hepatocellular carcinoma (HCC), nonalcoholic steatohepatitis (NASH), and the like.

In some embodiments, the synthetic ionizable phospholipids, LNPs, pharmaceutical compositions, and methods described herein may be used to treat a spleen disease or a spleen disorder. Non-limiting examples of spleen diseases and/or spleen disorders suitable for treatment via the methods herein can include hereditary spherocytosis, Gaucher disease, sickle cell disease, beta-thalassemia, and the like.

In some embodiments, the synthetic ionizable phospholipids, LNPs, pharmaceutical compositions, and methods described herein may be used to treat a skin disease or a skin disorder. Non-limiting examples of skin diseases and/or skin disorders suitable for treatment via the methods herein can include epidermolysis bullosa (EB), pachyonychia congenital, melanoma, ichthyosis, Hailey-Hailey disease, Sjögren-Larsson syndrome (SLS), xeroderma pigmentosum (XP), wound healing, netherton syndrome, and the like.

### V. Kits

The present disclosure also provides kits for use in delivering therapeutic agents, diagnostic agents, or gene editing systems to a target site (e.g., cell or tissue) or for treating/preventing a disease, disorder, and/or condition in a subject in need thereof. Such kits can include one or more containers comprising any of the pharmaceutical compositions described herein.

In some embodiments, the kit can comprise instructions for use in accordance with any of the methods described herein. The included instructions can comprise a description of administration of the pharmaceutical composition for delivering the therapeutic agents, diagnostic agents, or gene editing systems encapsulated therein or for treating a subject according to any of the methods described herein. The instructions relating to the use of the pharmaceutical composition described herein, which comprises a LNP, generally include information as to dosage, dosing schedule, and route of administration for the intended treatment.

The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits as described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, suitable packaging may be an autoinjector. Autoinjectors are one-use, disposable, spring-loaded syringes. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle).

The kits described herein may optionally provide additional components such as buffers and interpretive information. Normally, the kit comprises a container and a label or package insert(s) on or associated with the container. In some embodiments, the present disclosure provides articles of manufacture comprising contents of the kits described above.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1. A Library of iPhos was Rationally Designed with a Membrane Destabilizing Mechanism for Superior Endosomal Escape

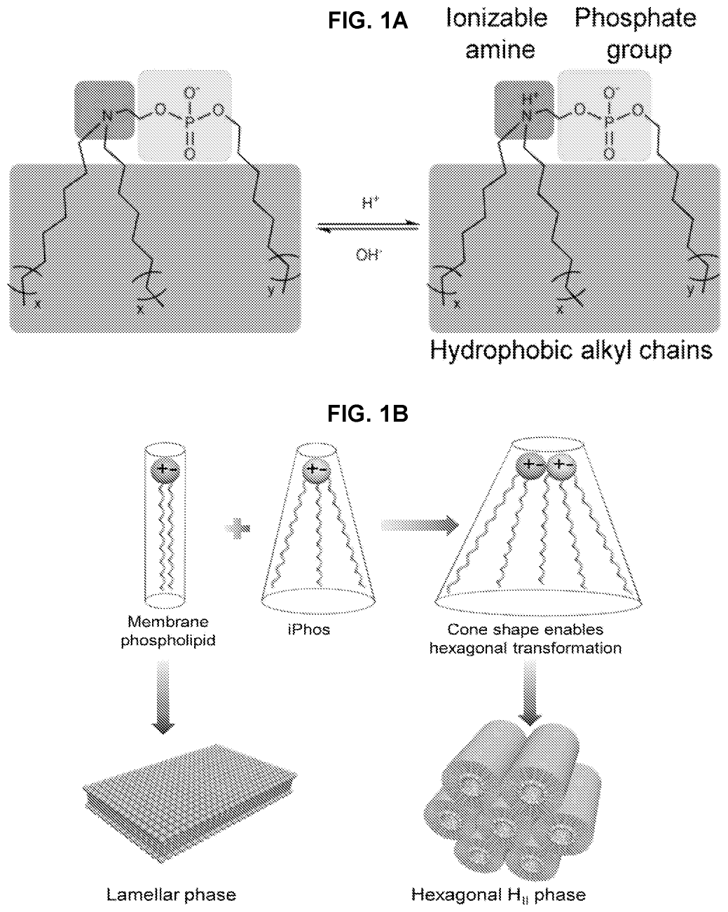

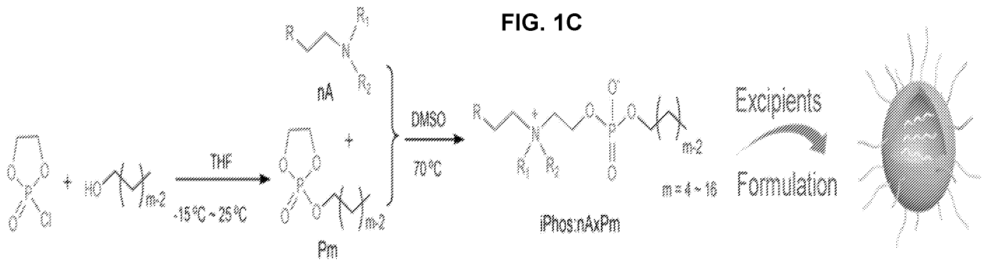

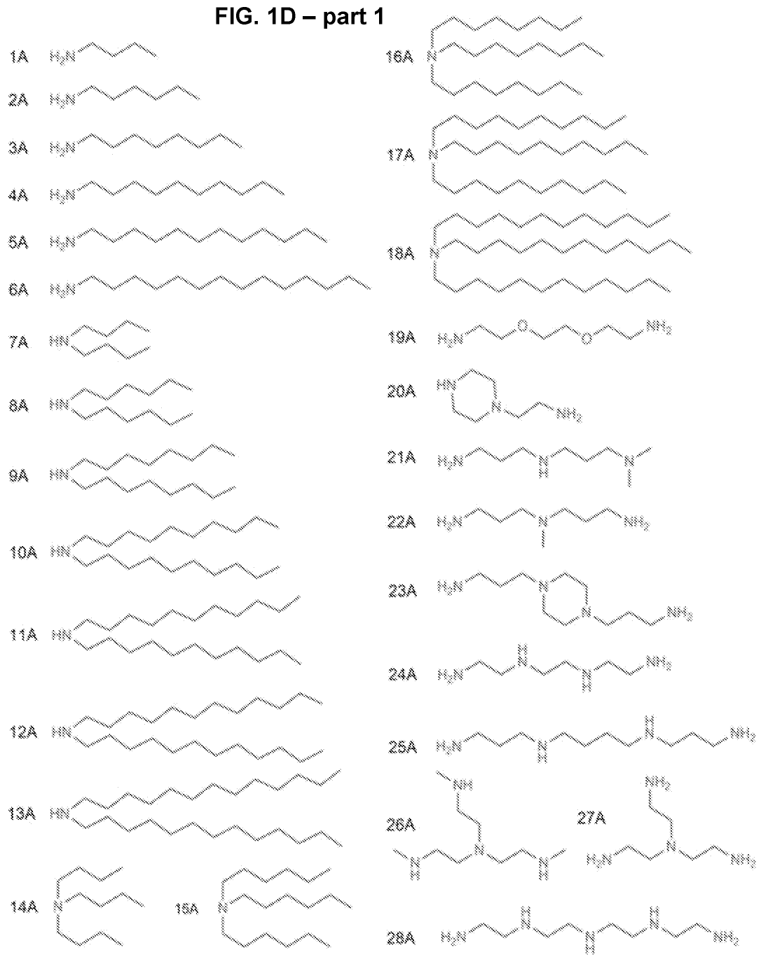

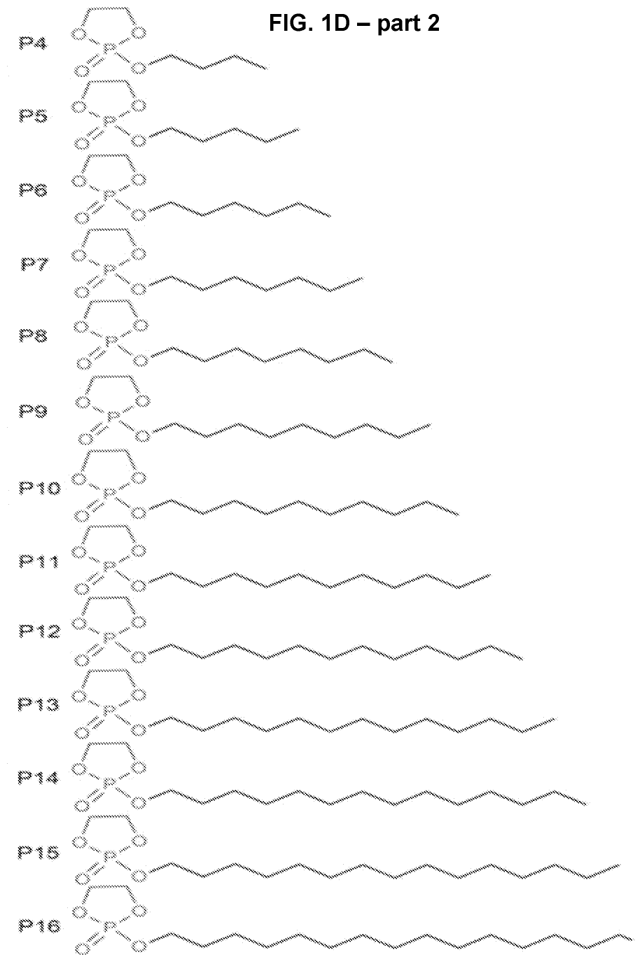

In accordance with the present disclosure, synthetic ionizable phospholipids (“iPhos lipids” or “iPhos”) which contained an ionizable amine, a phosphate group, and three hydrophobic tails was rationally designed. The small zwitterion constituted by amine and phosphate group was predicted to be reversible at different pHs. In physiological pH (˜7.4), the tertiary amine group will not be protonated, and the negatively charged iPhos will have difficulty fusing into the membranes. In contrast, upon entering the acidic endosomes, the tertiary amine was protonated to form a zwitterionic head (FIG. 1A). Upon studying the substrate scope, it was found that the three hydrophobic tail body was easier to mediate membrane phase transformation than that of two chains. The mechanism of action, therefore, was different from that of classic gene delivery carriers, because synthetic iPhos lipids can overall insert into natural phospholipid membranes, with preferable small ion pairs coupled with large tail body adopting a cone shape, to facilitate hexagonal H_{II} phase formation (FIG. 1B).

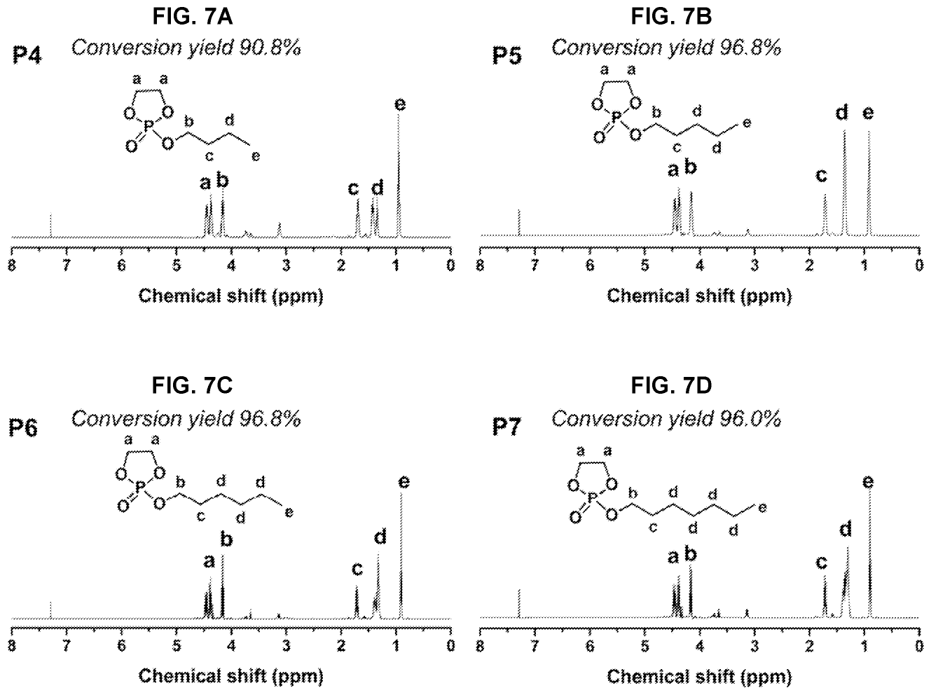

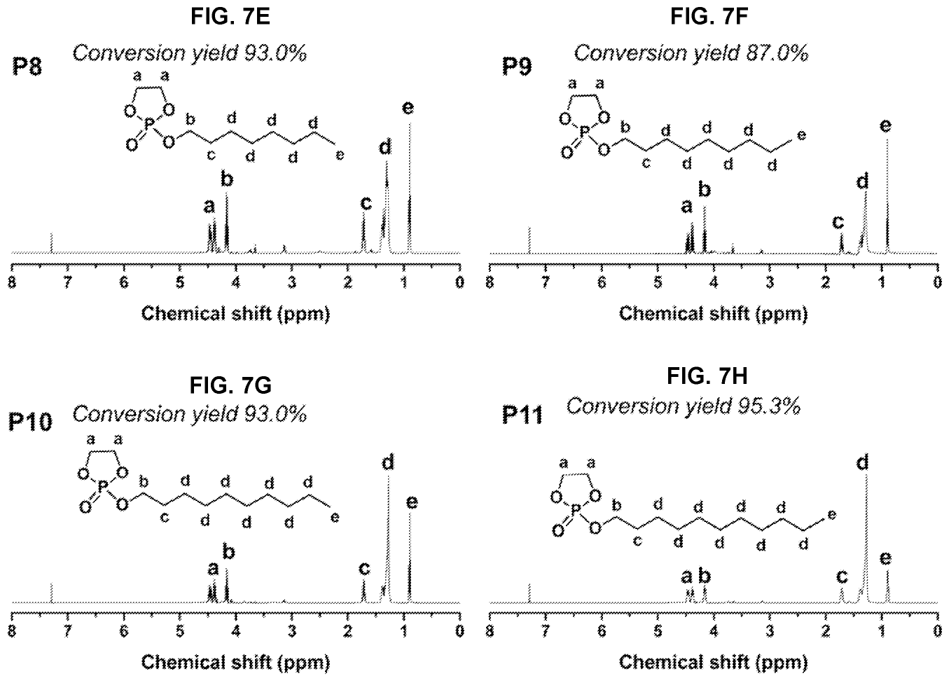

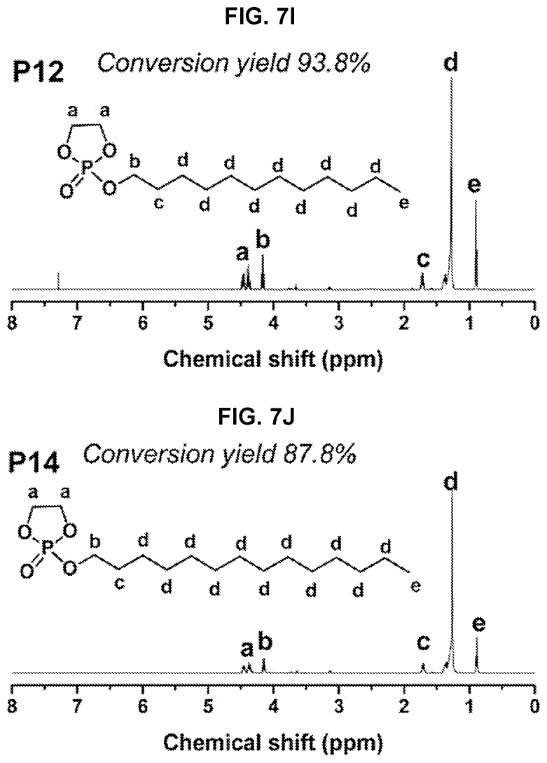

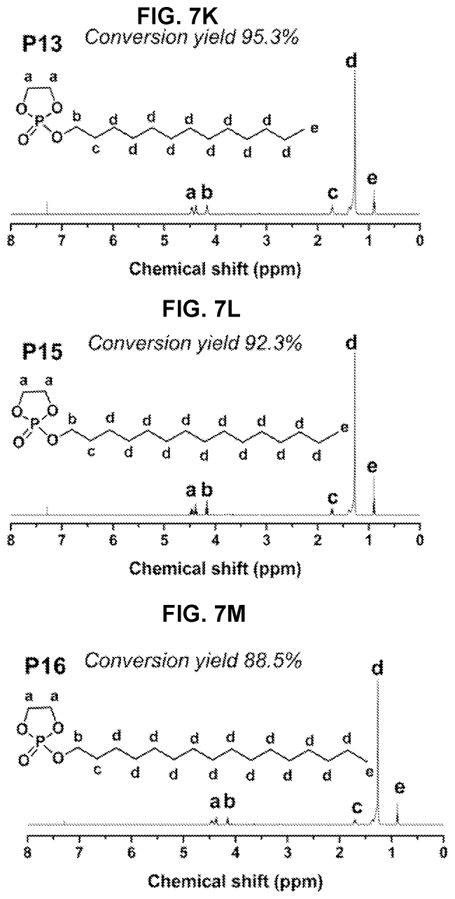

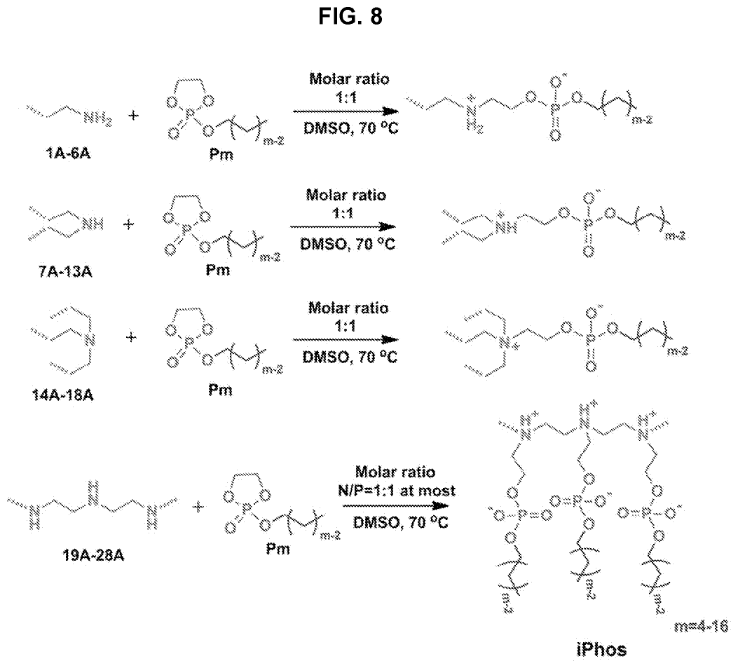

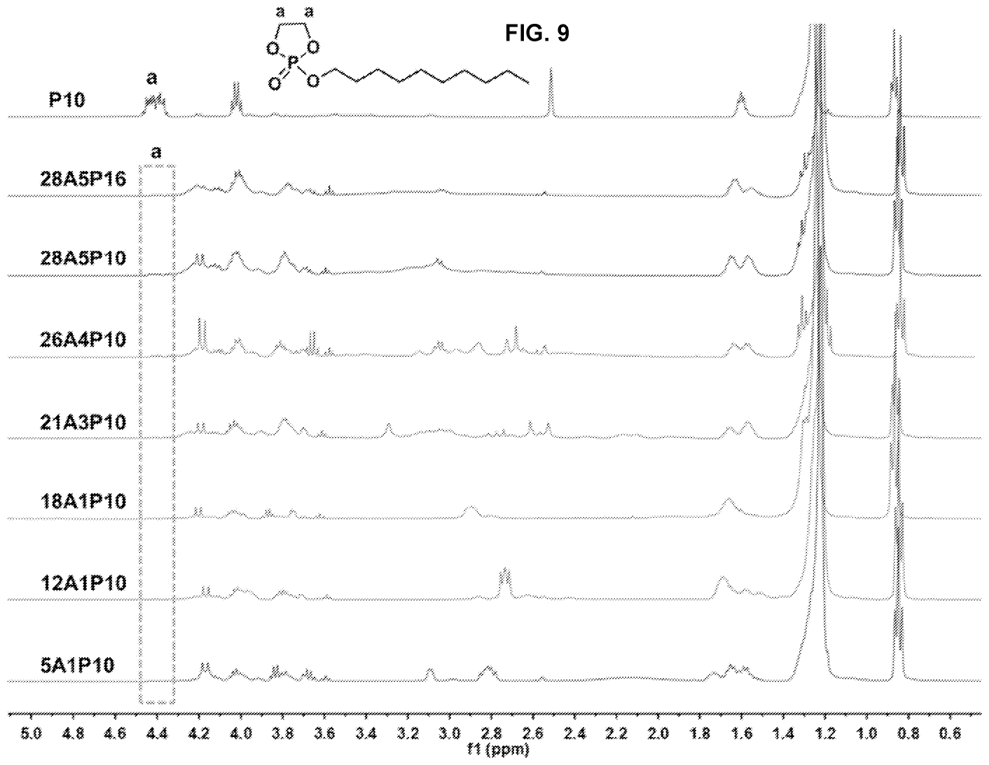

To overcome prior limitations in synthetic routes, we focused on ring-opening reactions that could yield diverse products with chemical complexity to meet the aforementioned design guidelines. The combinatorial reaction of amines (nA) with alkylated dioxaphospholane oxides (Pm) yielded 572 iPhos lipids (termed nAxPm) (FIG. 1C) where “x” indicated the number of Pm molecules modified on one amine molecule. Pm molecules were synthesized via esterification of 2-chloro-2-oxo-1,3,2-dioxaphospholane (COP) from corresponding alcohols with different alkyl chain lengths (FIGS. 7A-7M). Primary, secondary, and tertiary amine groups could all trigger Pm ring-opening to introduce different zwitterions (FIG. 8). To control the hydrophobic tail and zwitterion numbers, amines with different alkyl chain and amine group numbers were used (FIG. 1D and FIG. 9). The chemical design of iPhos was unique, because, zwitterion species (pH switchable and irreversible) became available in addition to group numbers through this strategy, greatly broadening the architectures and varieties of phospholipids.

### Example 2. In Vitro Screening Showed that Top iPhos Possessed a pH-Switchable Zwitterion and Three Tails

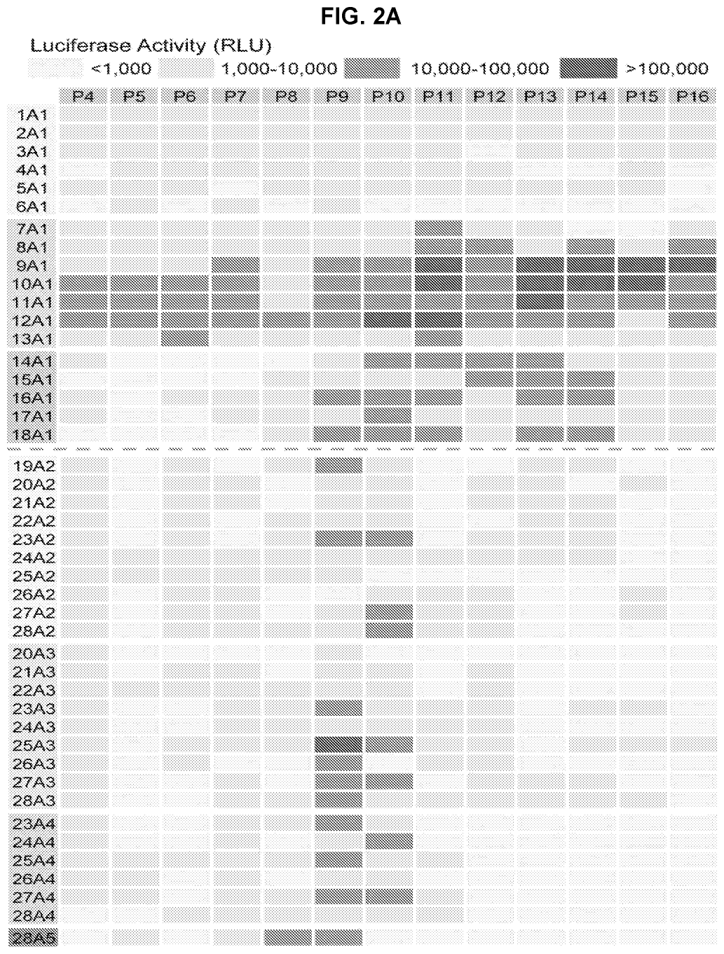

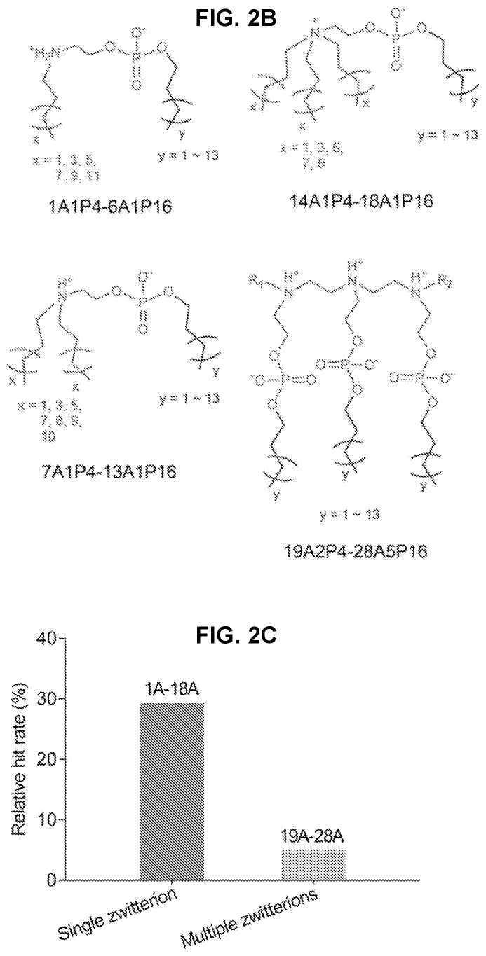

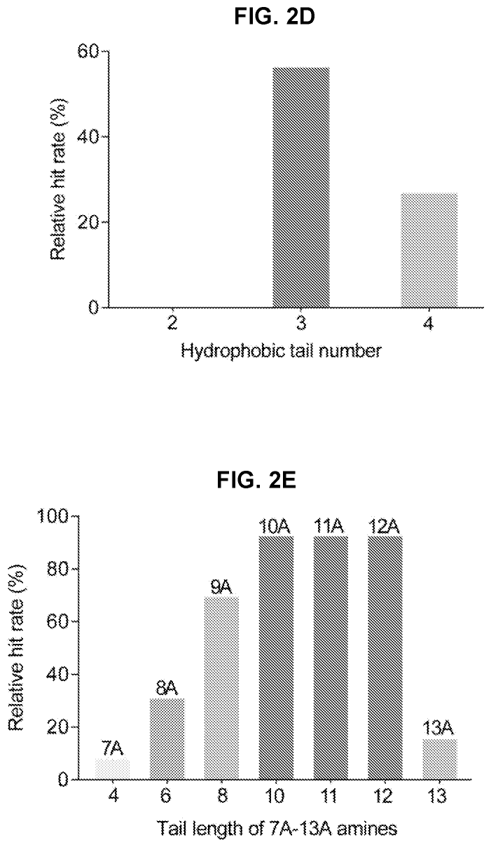

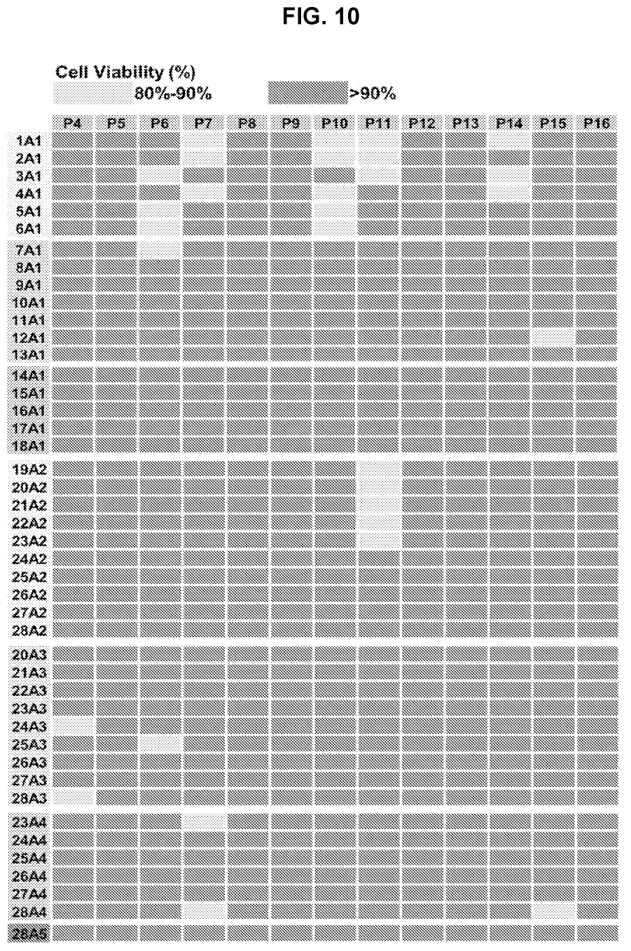

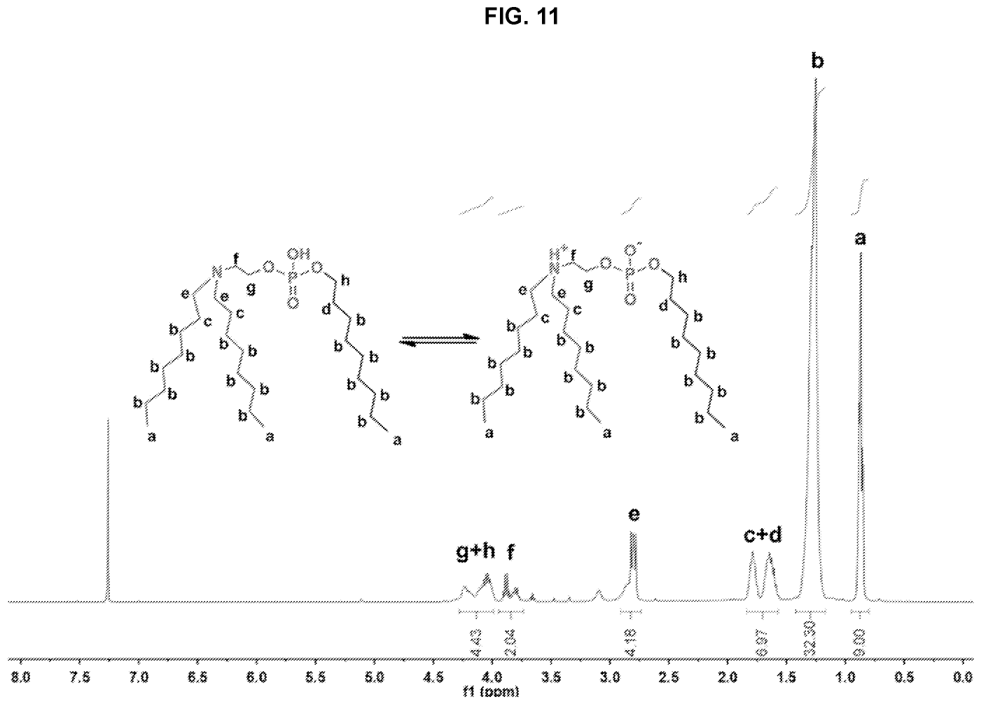

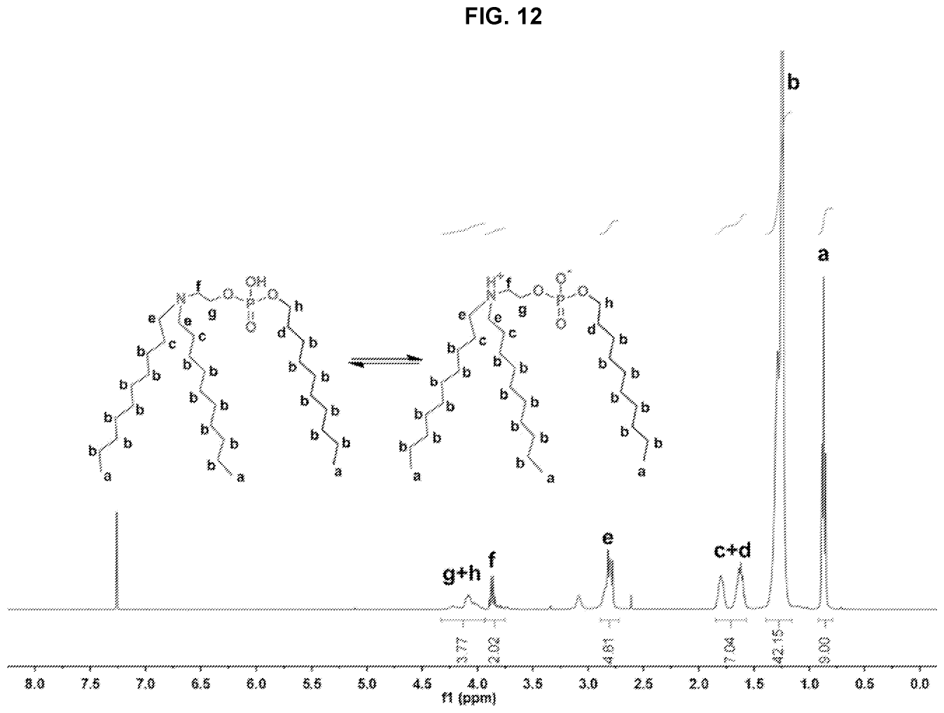

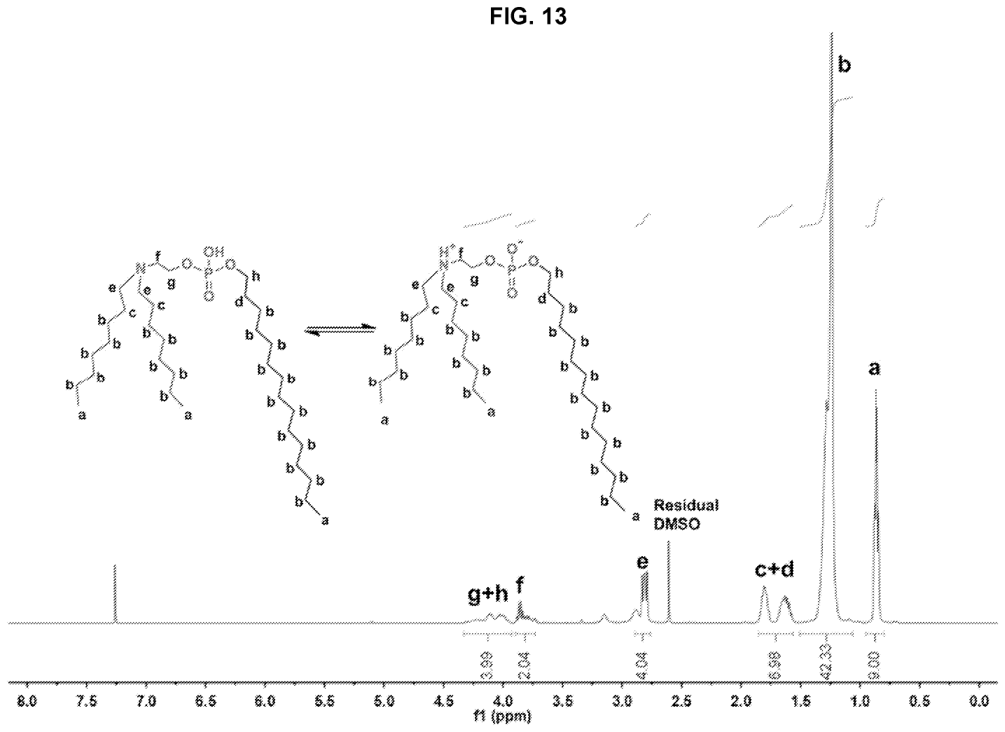

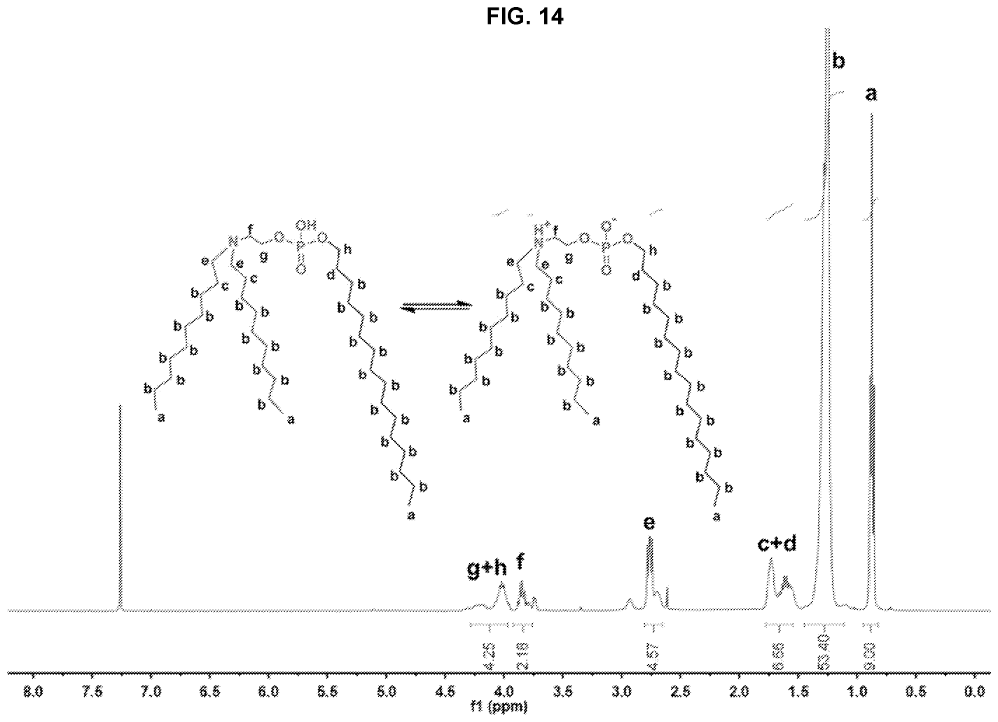

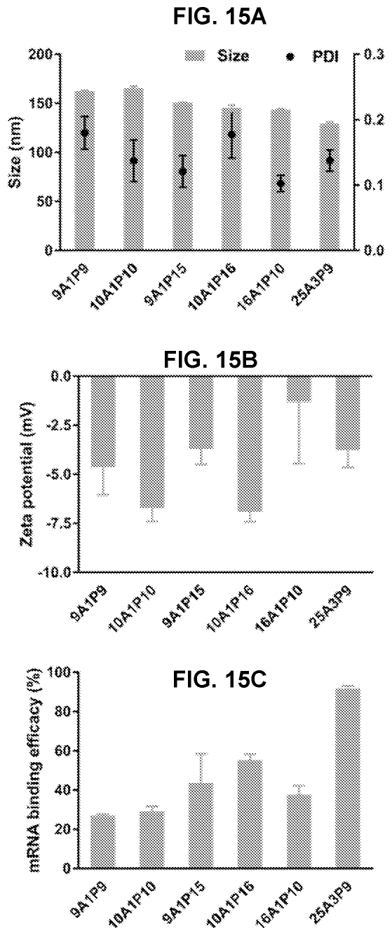

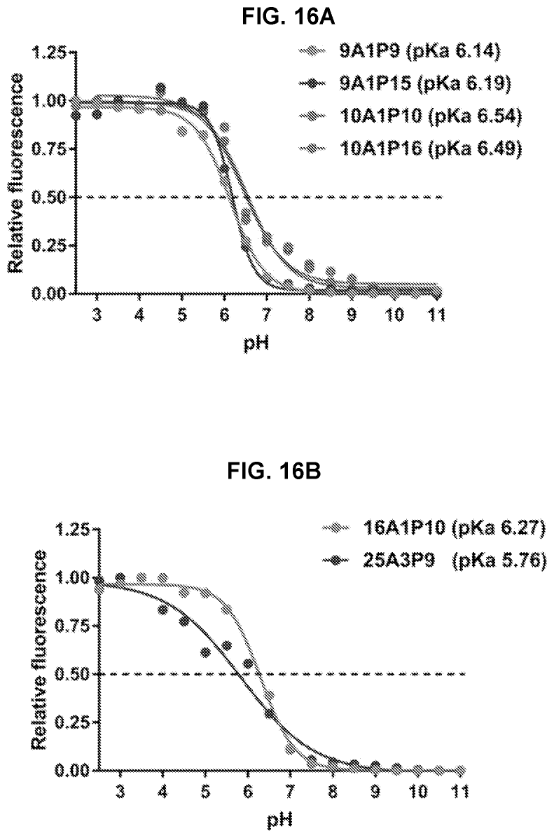

In accordance with the present disclosure, to evaluate the potential for mRNA delivery, iPhos lipid nanoparticles (iPLNPs) were used to transfect ovarian cancer cells IGROV-1. iPhos, helper lipid, chol, and 1,2-dimyristoyl-rac-glycero-3-methoxy(poly(ethylene glycol-2000)) (DMG-PEG2000) (25:30:30:1 mol/mol) were mixed to formulate iPLNPs using the ethanol dilution method. A structurally simple lipid, N-methyldioctadecylamine (MDOA), was first employed as the helper lipid to demonstrate the iPhos function in the initial screen. iPLNPs could thus be considered a different concept than traditional LNPs, where the modular emphasis was placed instead on the zwitterionic (functionally active iPhos) lipid and all other lipids become helper lipids. All initial iPhos lipids with different numbers and species of zwitterions and tails exhibited low toxicity (FIG. 10). From the in vitro screening heat map, it was concluded that iPhos with a single zwitterion (1A1P4-18A1P16) showed higher mRNA efficacy than that of multiple zwitterions (19A2P4-28A5P16) (FIGS. 2A-2C). The reason could be related to how multiple zwitterions constructed a larger head, making it difficult for membrane phase transformation. To further explore the SAR of iPhos with a single zwitterion, two tailed materials (1A1P4-6A1P16) exhibited far less efficacy, because the small tail body failed to formulate the cone shape with natural membrane phospholipids. iPhos 14A1P4-18A1P16 possessed a permanent zwitterion and lacked structural flexibility upon endosome internalization. Encouragingly, iPhos (7A1P4-13A1P16) composed of one tertiary amine, one phosphate group, and three hydrophobic tails showed the highest mRNA delivery efficacy as expected, with a hit rate around 60% (FIG. 2D). The small zwitterion head and large tail body promoted membrane fusion and phase transformation from lamellar to hexagonal H_{II}. Among these iPhos lipids, amine tail length was very important, and the hit rates of 10-12 chain lengths reached up to 92% (FIG. 2E). These observations were in contrast to previously reported ionizable amino lipid and lipidoid libraries, where efficacy generally correlated with polyamine cores and higher numbers of alkyl tails.²⁴⁻²⁶ These results indicated that iPhos lipids may operate by a different mechanism that ionizable amino lipids do. Next, some selected top iPhos lipids (9A1P9, 9A1P15, 10A1P10 and 10A1P16) were purified (FIGS. 11-14), and the resulting iPLNPs showed appropriate particle sizes (˜150 nm) for endocytosis, slightly negative surface zeta potentials (˜−5 mV) for serum protein resistance, as well as suitable pKa (6.0-6.5) for in vivo assays (FIGS. 15A-C and FIGS. 16A-16B). These capabilities impart synthetic iPhos lipids great potential for in vivo applications.

### Example 3. Model Membrane Studies Revealed the Mechanism of iPhos-Mediated Endosomal Rupture with Correlation to Chemical Architecture

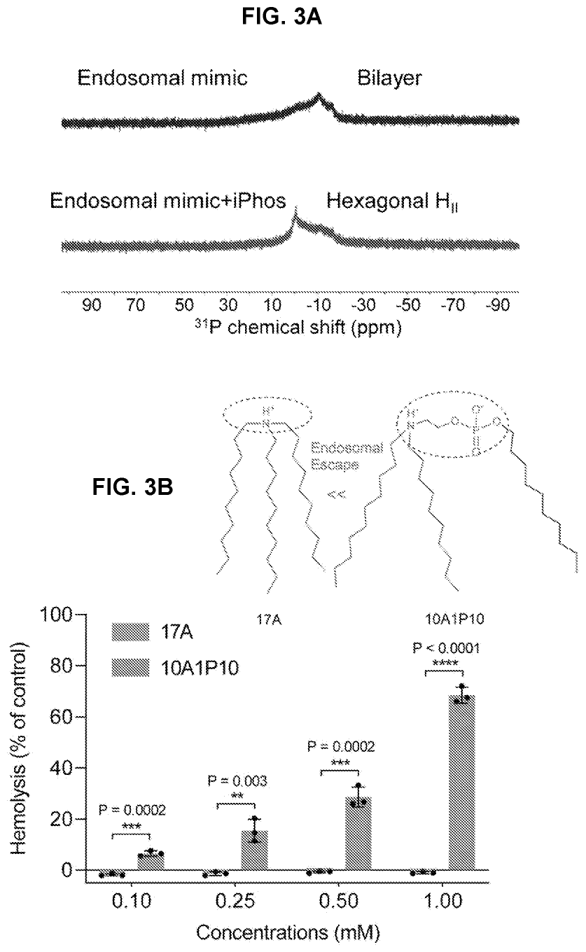

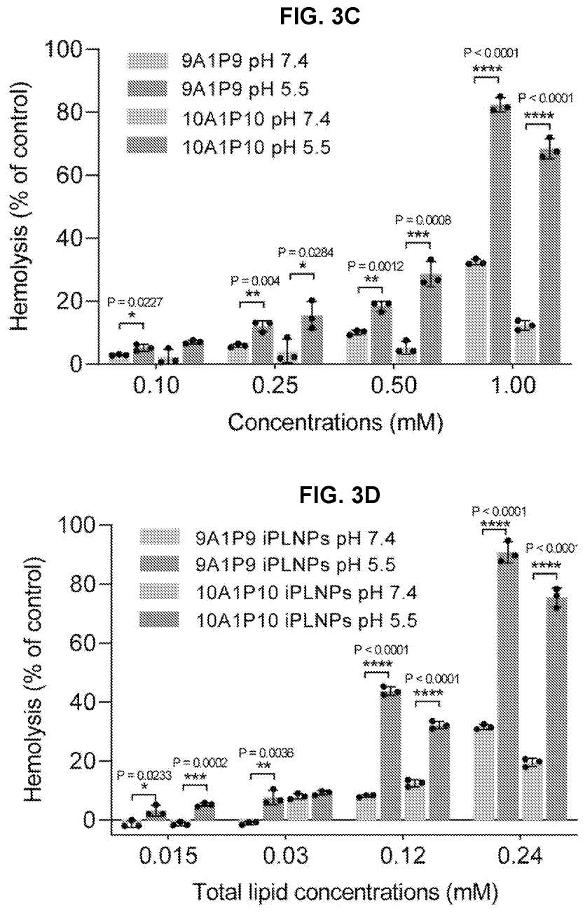

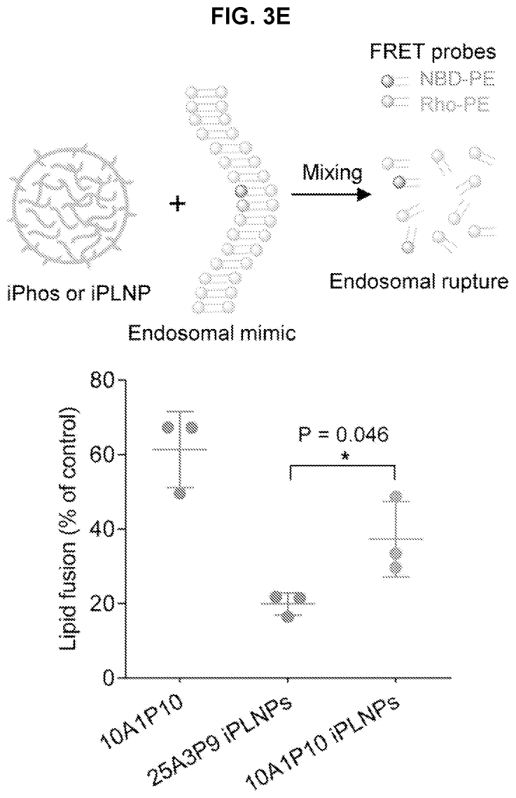

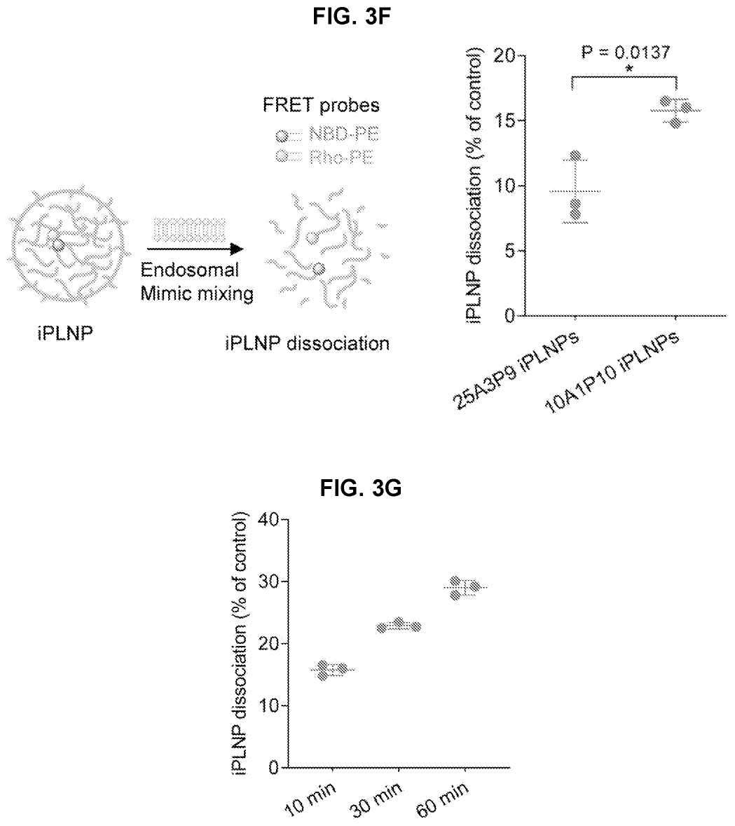

In accordance with the present disclosure, the membrane-disruptive activity of iPhos lipids and iPLNPs was first evaluated by a hemolysis model.^{23, 27} Top iPhos lipids (9A1P9 and 10A1P10) containing a pH-switchable zwitterion head and three tails were examined. 10A1P10 exhibited dramatically higher hemolysis than 17A with a simple tertiary amine, validating the superiority of zwitterion in membrane fusion and rupture (FIG. 3A). Also, 9A1P9, 10A1P10, and related iPLNPs showed higher membrane-disruptive activity at acidic endosomal compartment compared to that of neutral physiological environment (FIGS. 3B-3C).

Following this, a fluorescence resonance energy transfer (FRET) assay was utilized to evaluate iPhos lipid membrane fusion and iPLNP dissociation. Two DOPE-conjugated FRET probes, 7-nitrobenzo-2-oxa-1,3-diazole (NBD-PE) and lissamine rhodamine B (Rho-PE), were formulated into a single endosome mimicking liposome, leading to attenuated NBD fluorescence because of FRET to rhodamine. Once lipid fusion occurred, the resulting larger distance between the two probes gave rise to NBD signal increase.²⁸ As shown in FIG. 3D, 10A1P10 iPLNPs exhibited higher lipid fusion than 25A3P9 iPLNPs, demonstrating that a small single zwitterion head showed stronger trend to insert and destroy endosomal membranes compared to that of multiple zwitterions. Afterward, FRET probes were further used to study iPLNPs, and 10A1P10 iPLNPs were easier to disassemble to release mRNA than 25A3P9 iPLNPs once mixing with endosome mimicking liposomes (FIGS. 3E-3G). These results demonstrated that apart from a large tail body, a pH-switchable small zwitterion head in iPhos lipids is essential for endosomal escape.

### Example 4. In Vivo SAR of iPhos Demonstrated that the Chemical Structure and Alkyl Length Controlled Efficacy and Organ Selectivity

In accordance with the present disclosure, due to the additional barriers for in vivo delivery, not all known carriers with in vitro activity translated to animal models.^{29, 30} Moreover, comparing siRNAs/miRNAs (18-22 bp) to long mRNAs (1,000-6,000 nt), weaker electrostatic association was required, allowing for mRNA release post cellular internalization.¹² Therefore, the chemistry of iPhos lipids may have an inherent advantage over cationic lipids and play a pivotal role in mRNA delivery systems.

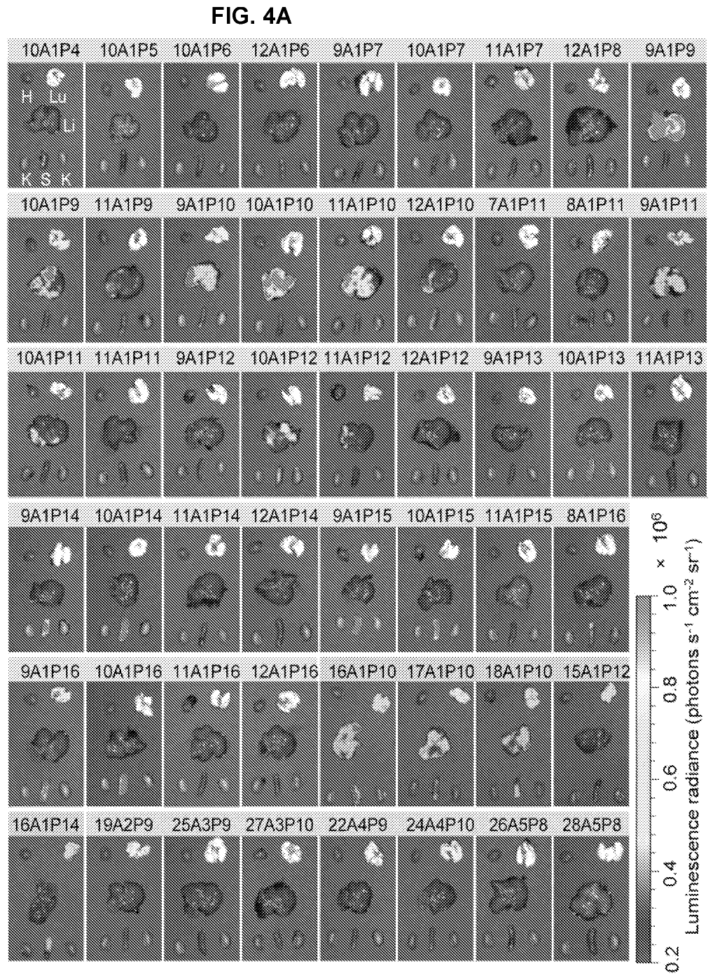

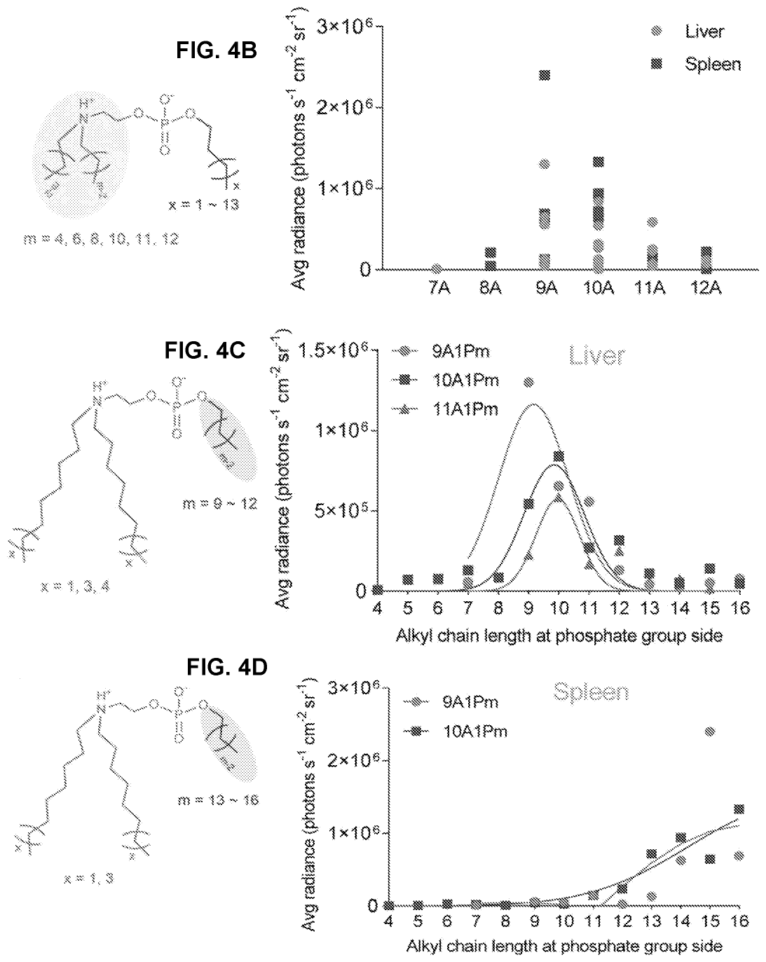

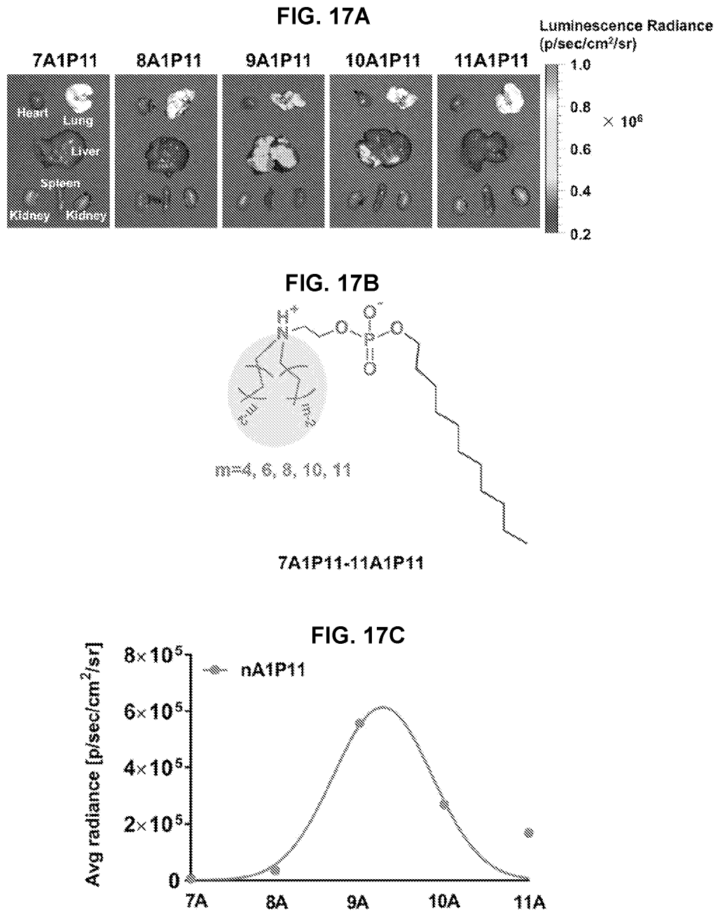

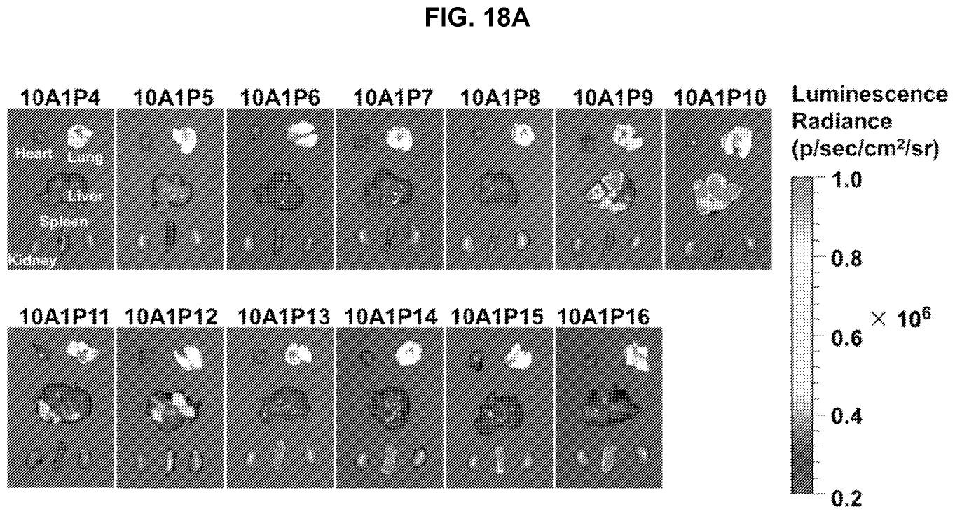

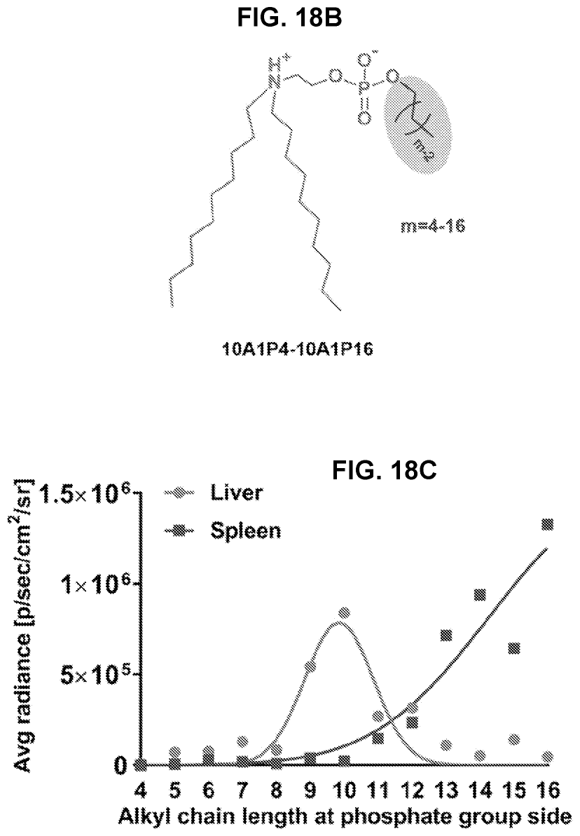

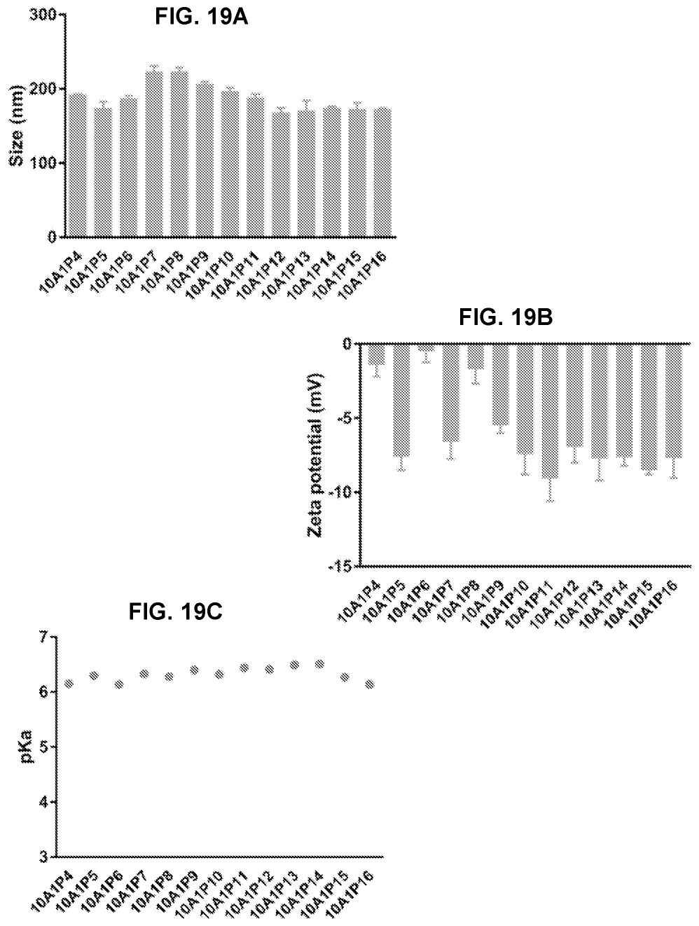

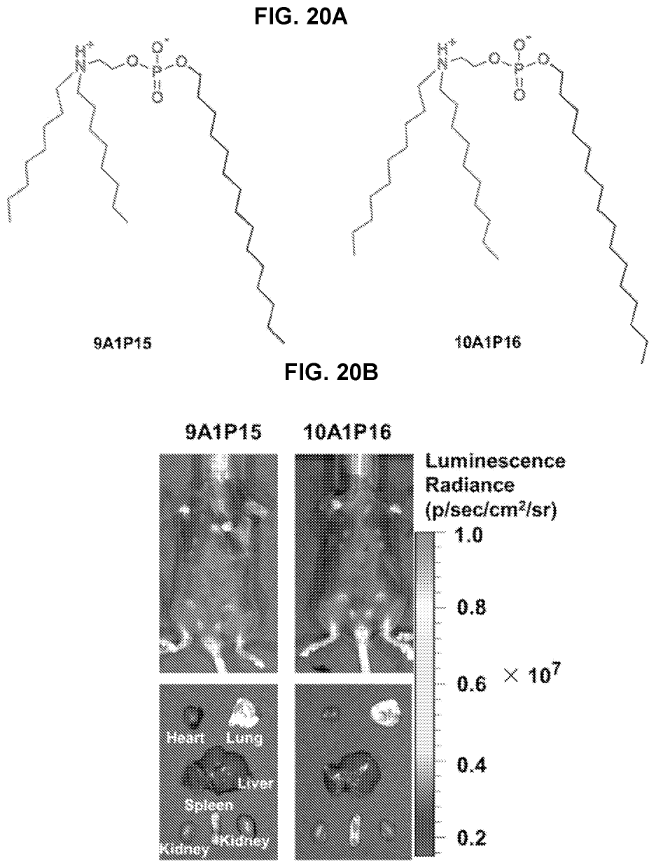

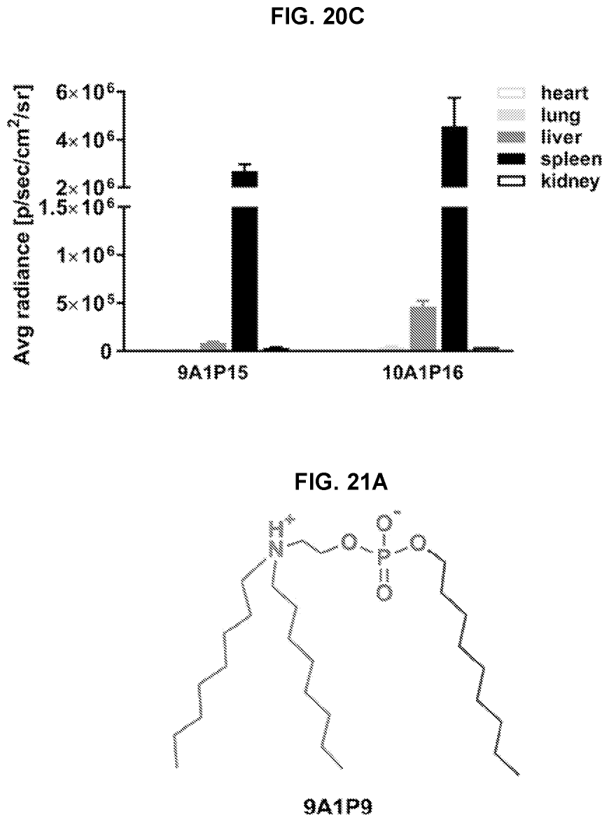

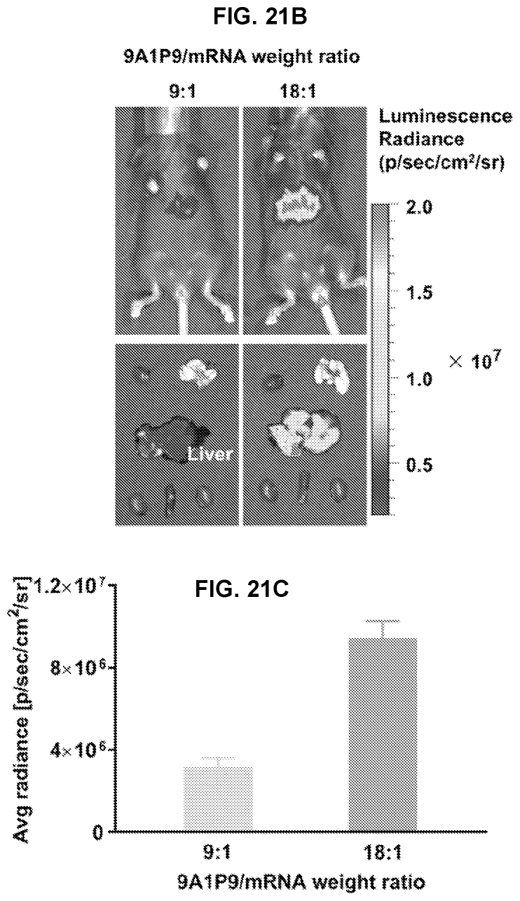

51 efficient iPhos lipids were selected from the in vitro screen and evaluated in vivo delivery at the low mRNA dose of 0.1 mg kg⁻¹ (FIG. 4A). iPhos lipids containing multiple zwitterions failed to delivery mRNA in vivo. Further establishing SAR, iPhos lipids with one tertiary amine, one phosphate group, and three alkyl tails were the most efficacious. Interestingly, alkyl chain length tremendously affected efficacy and organ selectivity. Chain length at amine side determined efficacy, and eight to ten carbon lengths mediated high in vivo mRNA expression (FIG. 4B and FIG. 17). Surprisingly, it was discovered that alkyl length beside phosphate group influenced mRNA transfection in selective organs (FIGS. 4C-4D). Shorter chains (9-12 carbons) showed mRNA translation in liver, while longer chains (13-16 carbons) transferred protein expression to spleen. In vivo assessment of 10A1P4-10A1P16 also clearly supported this inference (FIG. 18). Following this, nanoparticle size, zeta potential, and pKa of these iPLNPs were evaluated, where no obvious differences were observed (FIG. 19). Then iPhos based iPLNPs were evaluated at a higher dose (mRNA, 0.25 mg kg⁻¹) (FIGS. 20 and 21). Still, organ selectivity was achieved that 9A1P9 iPLNPs showed main mRNA expression in liver, while 9A1P15 and 10A1P16 iPLNPs mediated mRNA translation in spleen. The SAR provided a guideline to develop other efficacious vector materials with organ selectivity and specificity.

### Example 5. Synthetic iPhos Lipids Showed Broad Compatibility with Various Helper Lipids to Mediate Tissue-Selective Gene Delivery and Editing

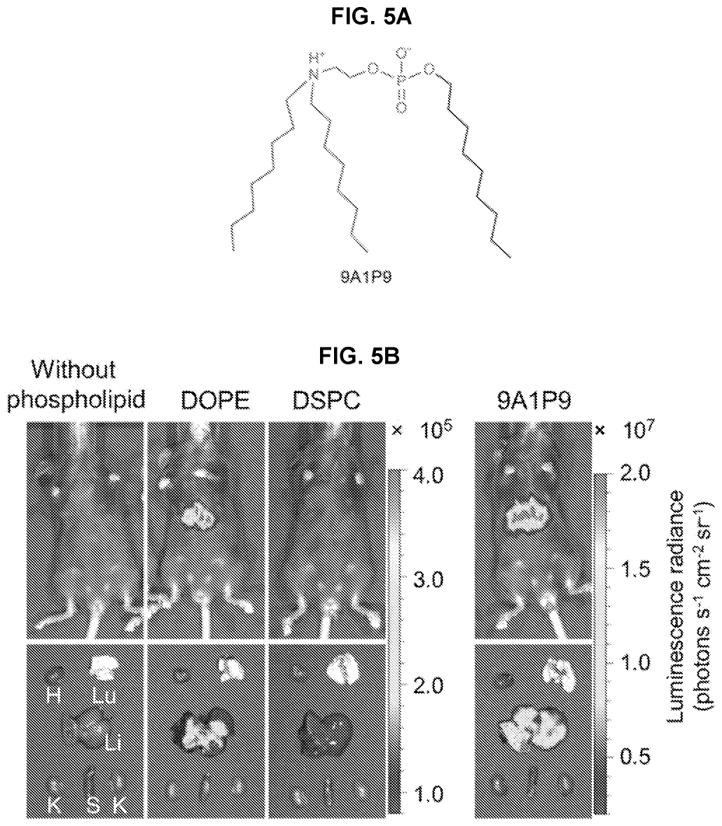

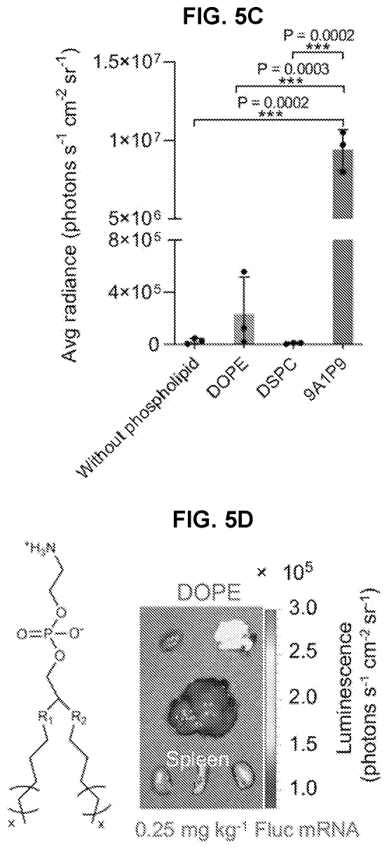

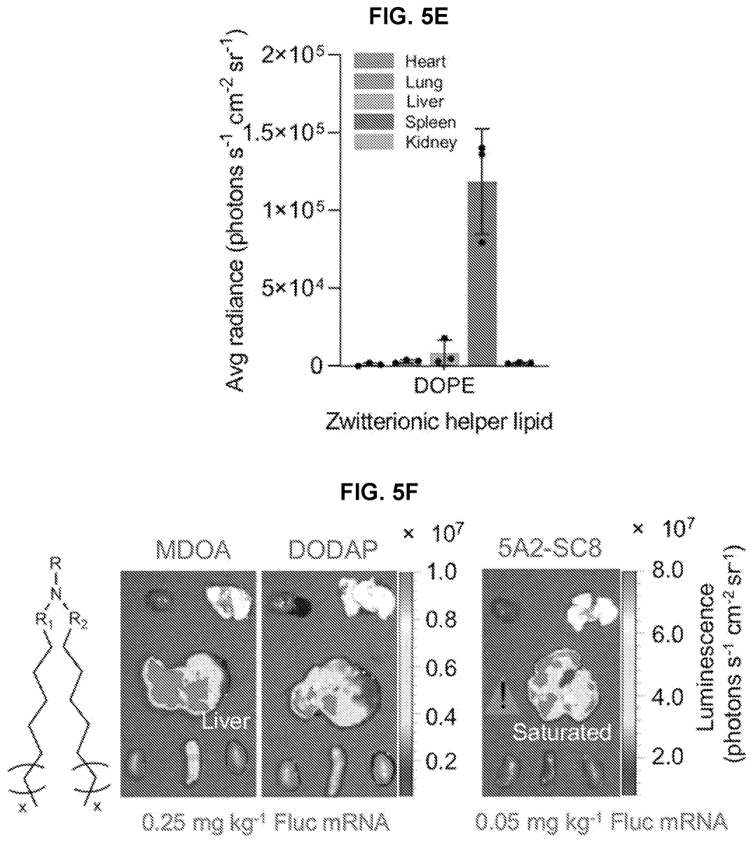

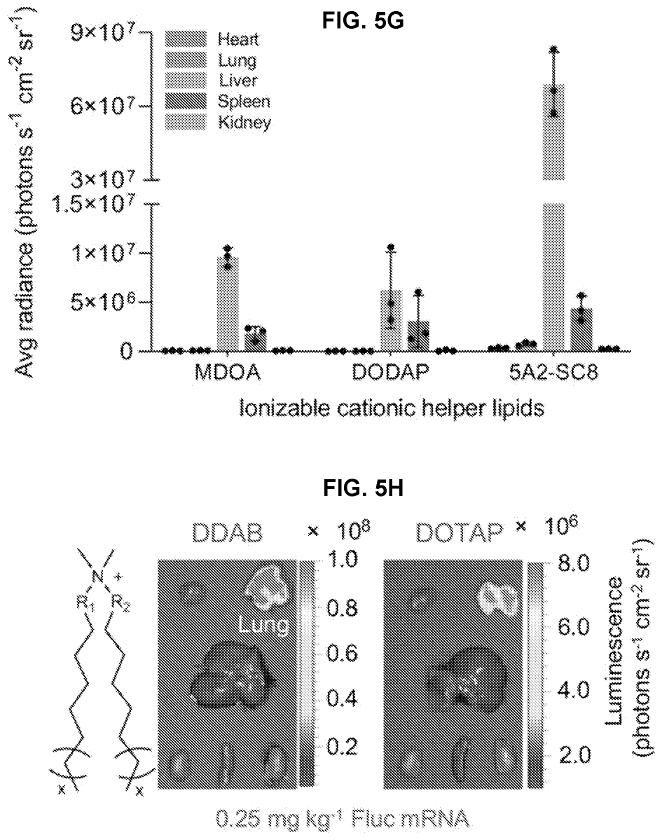

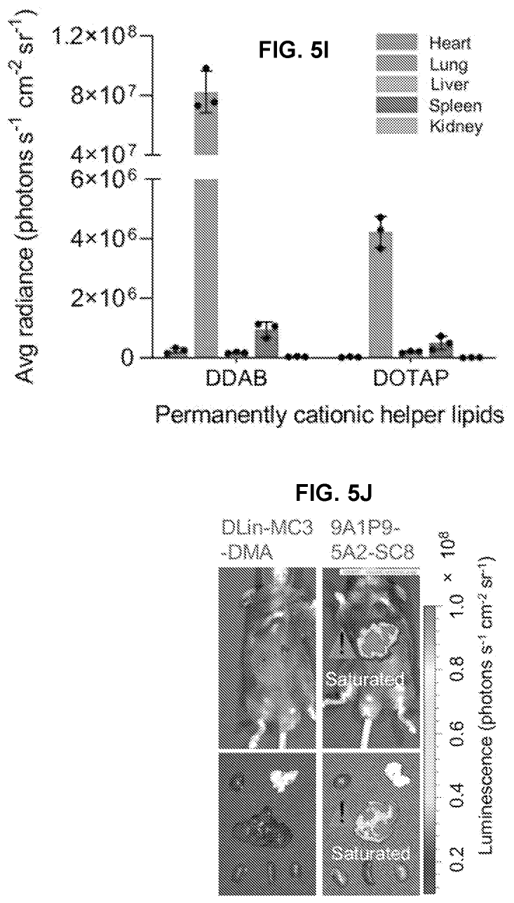

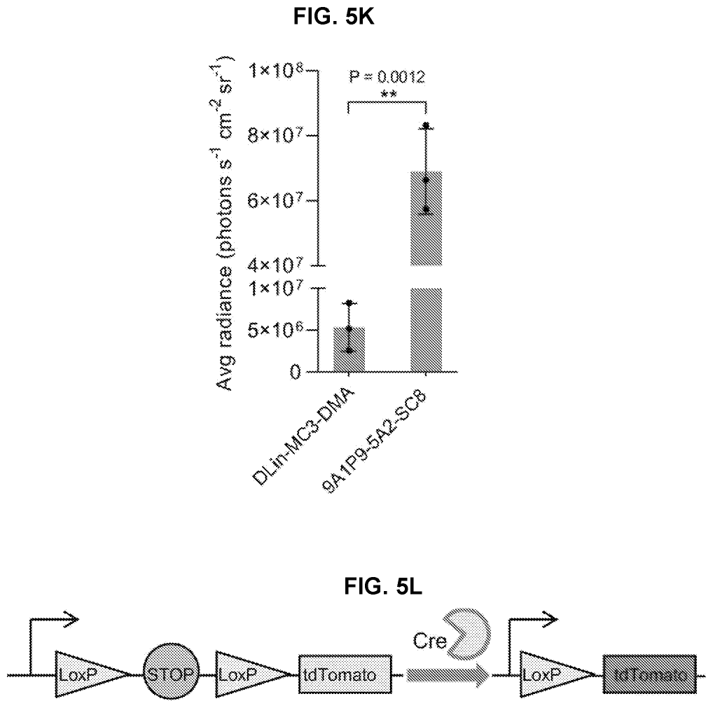

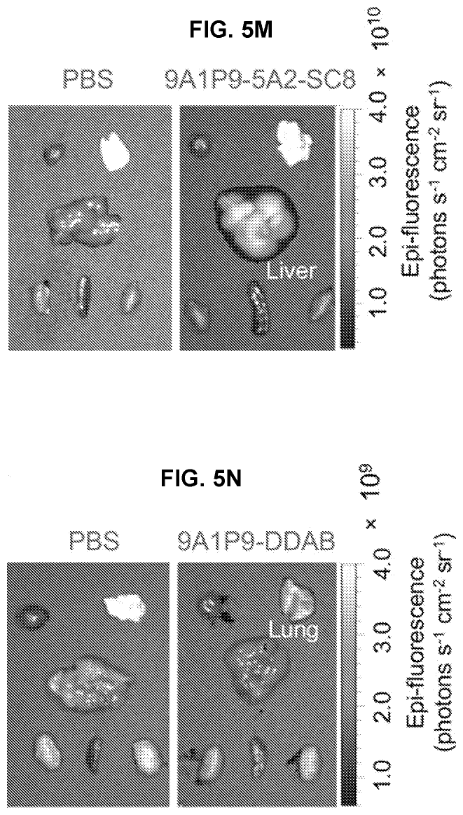

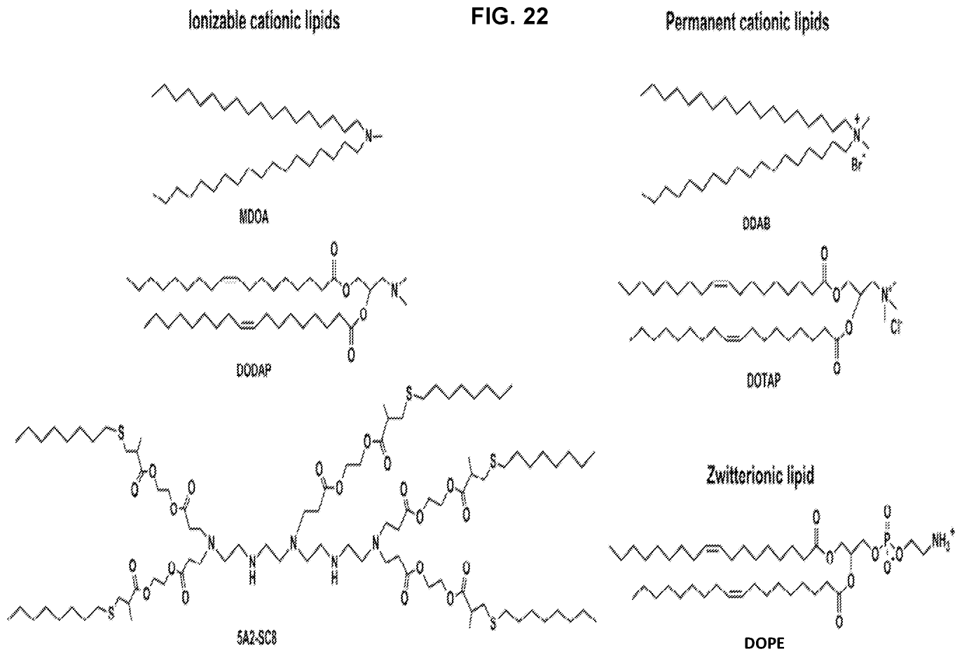

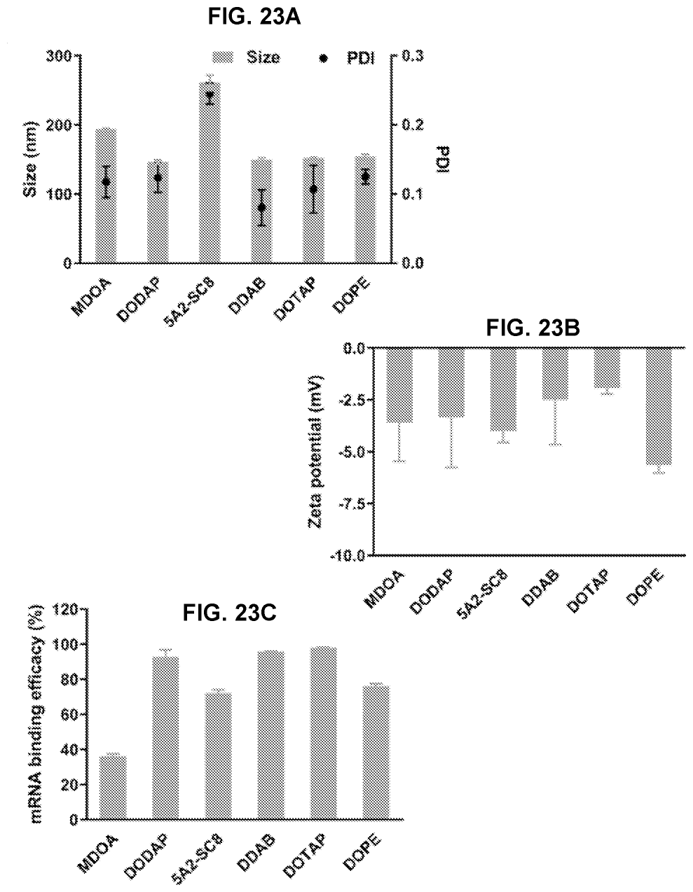

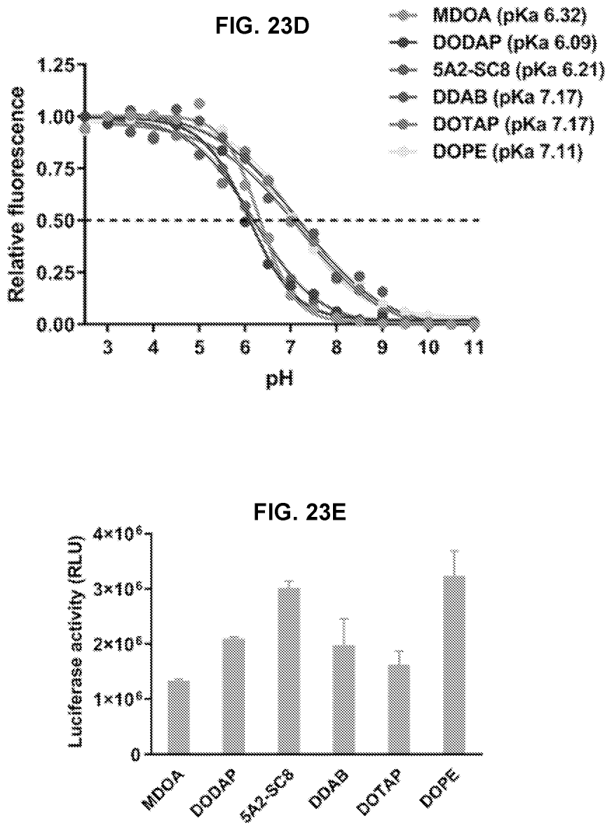

In accordance with the present disclosure, top-performing lipid 9A1P9 was initially identified when employed in an iPLNP containing the simple helper lipid MDOA. To confirm that iPhos 9A1P9 was the most important and active component of iPLNPs, a series of experiments was conducted. First, it was found that 9A1P9 exhibited 40- to 965-fold higher in vivo efficacy compared to the best currently used phospholipids DOPE and DSPC (FIGS. 5A-5C). Second, a variety of other established lipids were assessed in our 9A1P9 iPLNP mRNA delivery system as helper lipids to show the broad applicability. Zwitterionic lipids (DOPE), ionizable cationic lipids (MDOA, DODAP, and 5A2-SC8²⁶), and permanently cationic lipids (DDAB and DOTAP) were investigated as helper lipids (FIG. 22). The molar ratios of compositions were determined by orthogonal design methodology^{12, 31} and shown in Table 1. All formulated iPLNPs exhibited appropriate diameters, zeta potentials, mRNA binding, pKa, and high in vitro mRNA delivery efficacy (FIG. 23).

TABLE 1
iPLNP compositions and ratios for organ selective mRNA expression.

| | Molar ratios | | | | 9A1P9/ |
|---|---|---|---|---|---|
| | | Helper | | DMG- | mRNA |
| Helper lipid | 9A1P9 | lipid | Chol | PEG2000 | (w/w) |
| DOPE | 55 | 30 | 45 | 0.2 | 18/1 |
| MDOA | 25 | 30 | 30 | 1 | 18/1 |
| DODAP | 25 | 30 | 30 | 1 | 18/1 |
| 5A2-SC8 | 25 | 30 | 30 | 1 | 18/1 |
| DDAB | 60 | 30 | 40 | 0.4 | 18/1 |
| DOTAP | 60 | 30 | 40 | 0.4 | 18/1 |

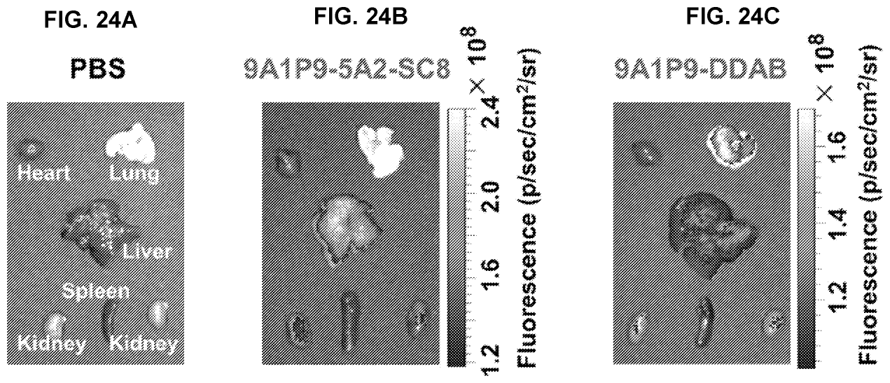

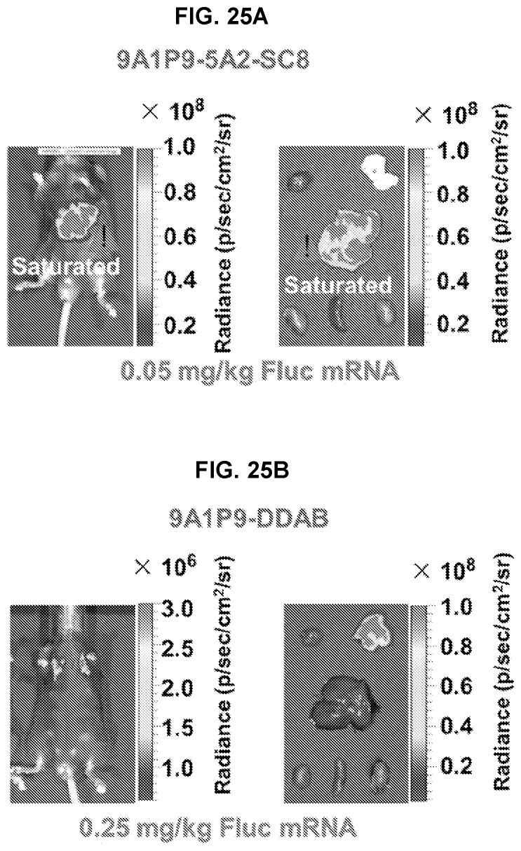

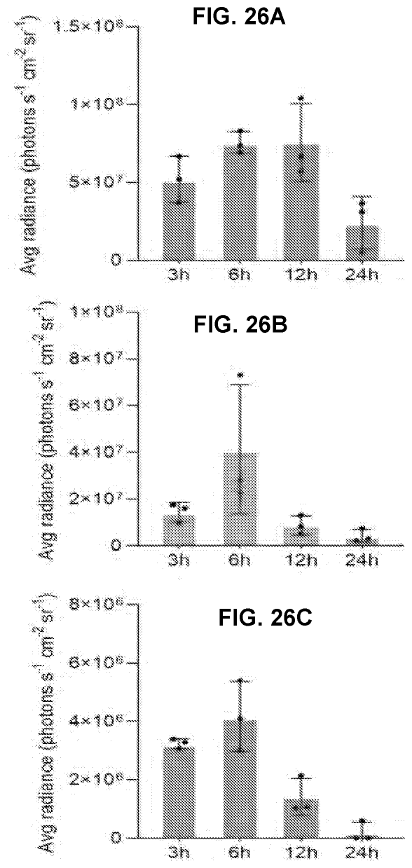

9A1P9 coupled with different helper lipids achieved organ selectivity. 9A1P9 iPLNPs with zwitterionic, ionizable cationic, and permanently cationic helper lipids enabled selective mRNA expression in spleen, liver, and lungs, respectively (FIGS. 5D-5I). Two highly efficacious formulations were further studied and in vivo biodistribution results revealed that 9A1P9-5A2-SC8 and 9A1P9-DDAB iPLNPs mediated high accumulation in liver and lung, respectively (FIGS. 24A-24C). Since iPhos lipids could enhance already efficacious formulations in a modular fashion, it was determined that 9A1P9-5A2-SC8 and 9A1P9-DDAB combinations exhibited mRNA expression of ultra-high level specifically in liver (˜10⁸ photons s⁻¹ cm⁻² sr-⁻¹, 0.05 mg kg⁻¹) and lung (˜10⁸ photons s⁻¹ cm⁻² sr⁻¹, 0.25 mg kg⁻¹), respectively (FIGS. 5F-5I and FIG. 25). Delivering Cre-recombinase mRNA (Cre mRNA), high efficacy and organ selectivity were still retained (FIGS. 5J-5L). Compared to “gold standard” DLin-MC3-DMA (used in FDA-approved Onpattro) LNPs, 9A1P9-5A2-SC8 iPLNP still showed 13-fold higher mRNA delivery efficacy in vivo (FIGS. 5M-5N). Therefore, iPLNPs were different from traditional cationic lipid LNPs, and high efficacy as well as controllable organ selectivity were both accomplished. Kinetic analysis revealed that protein expression occurred rapidly and peaked at around 6 hours post injection (FIGS. 26A-26C).

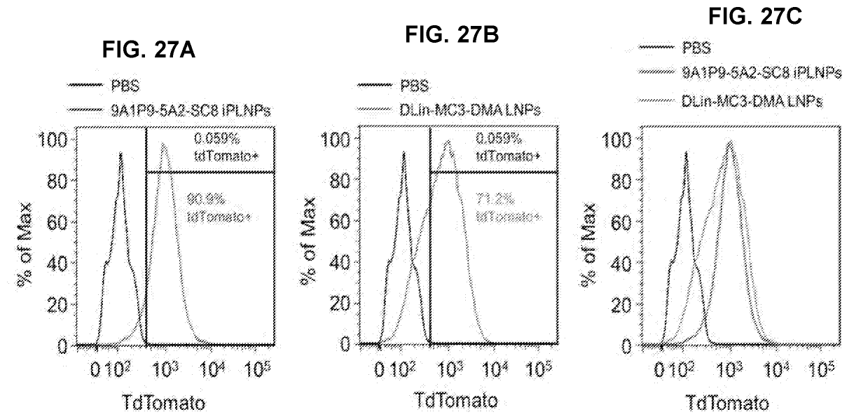

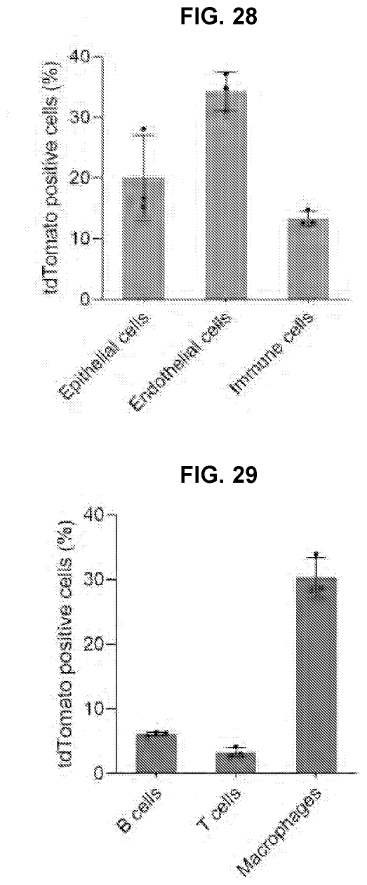

This model was next used to quantify transfection of specific cell types in liver, lung, and spleen organs. Following delivery of Cre mRNA, liver-selective 9A1P9-5A2-SC8 iPLNPs mediated mRNA delivery to ˜91% of all hepatocytes (FIGS. 27A-27C). Lung-selective 9A1P9-DDAB iPLNPs transfected ˜34% of all endothelial cells, ˜20% of all epithelial cells and ˜13% of immune cells (FIG. 28). Spleen-selective 10A1P16-MDOA iPLNPs transfected ˜30% of all macrophages and 6% of all B cells (FIG. 29). iPLNP proposed here represented one of the most efficacious mRNA delivery systems and held great potential for organ selective CRISPR/Cas9 gene editing.

### Example 6. 9A1P9 iPLNPs Achieved Liver or Lung Selective CRISPR/Cas9 Gene Editing

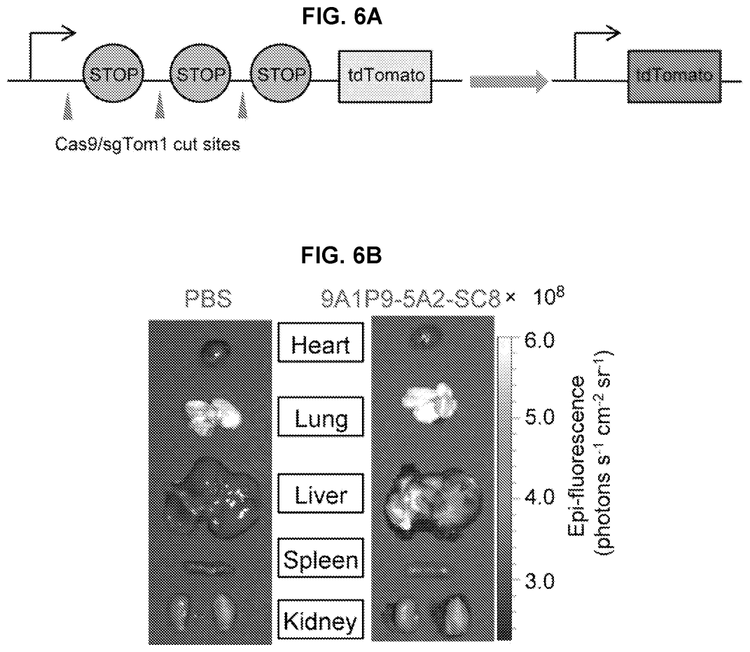

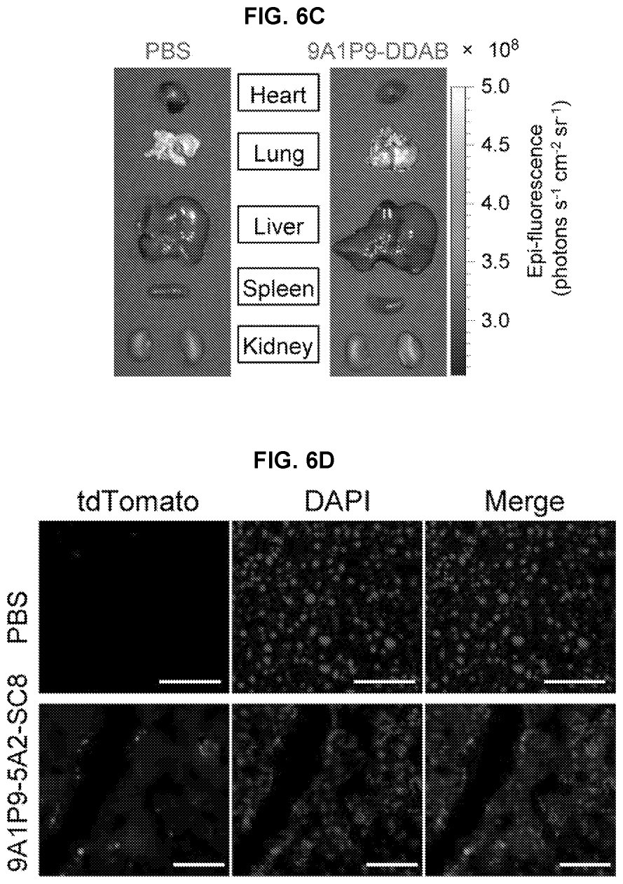

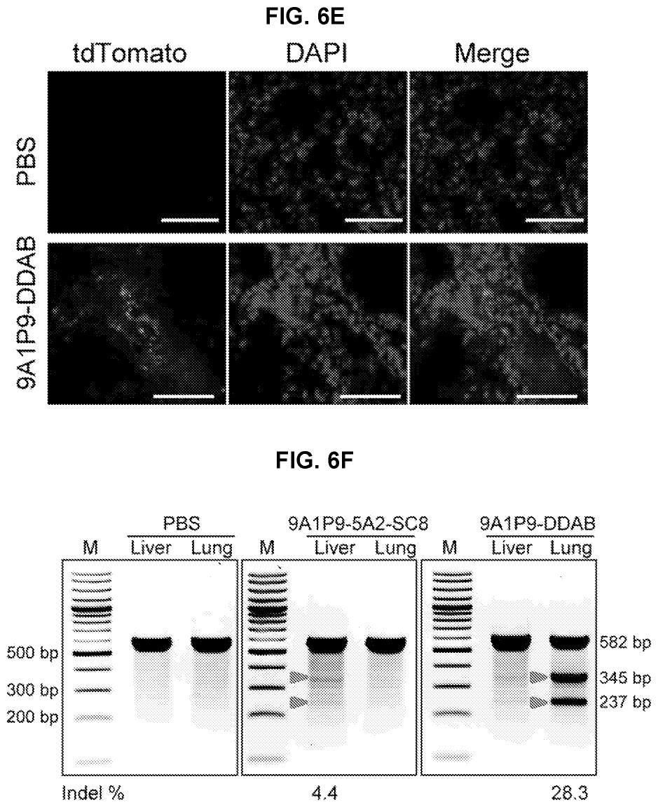

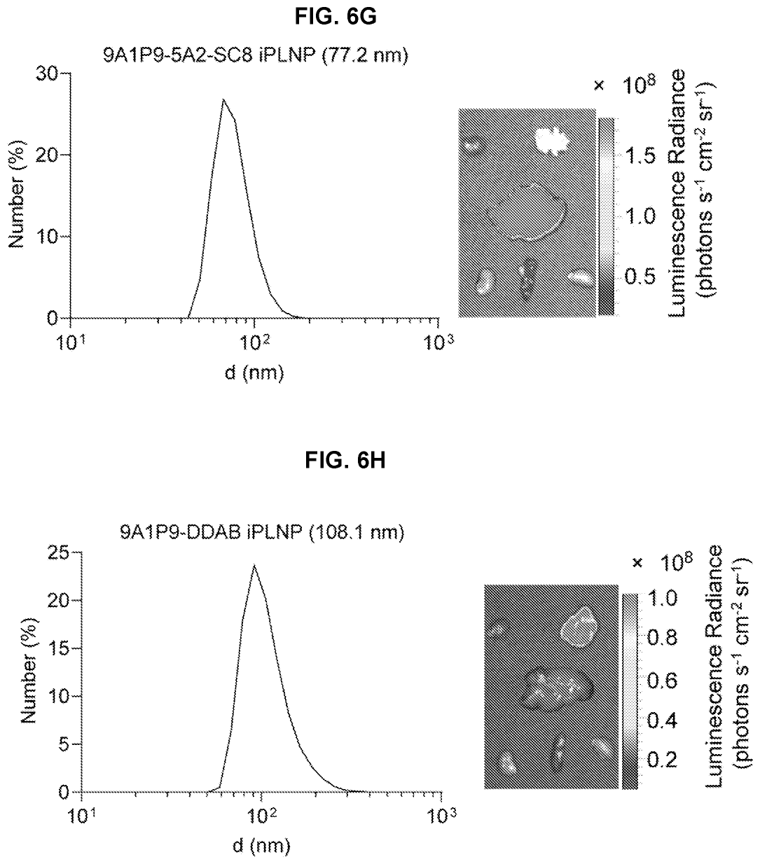

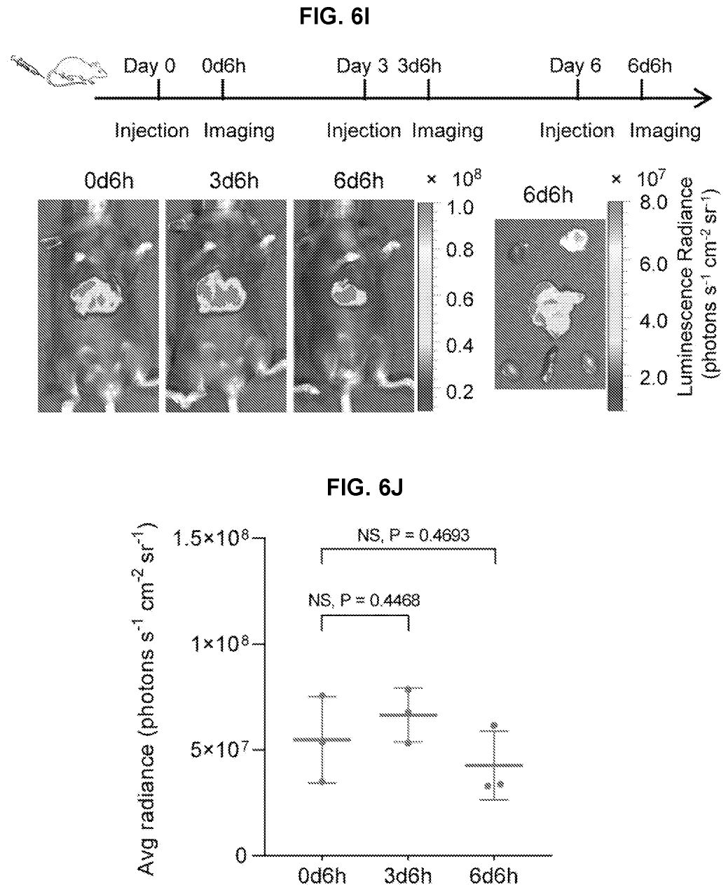

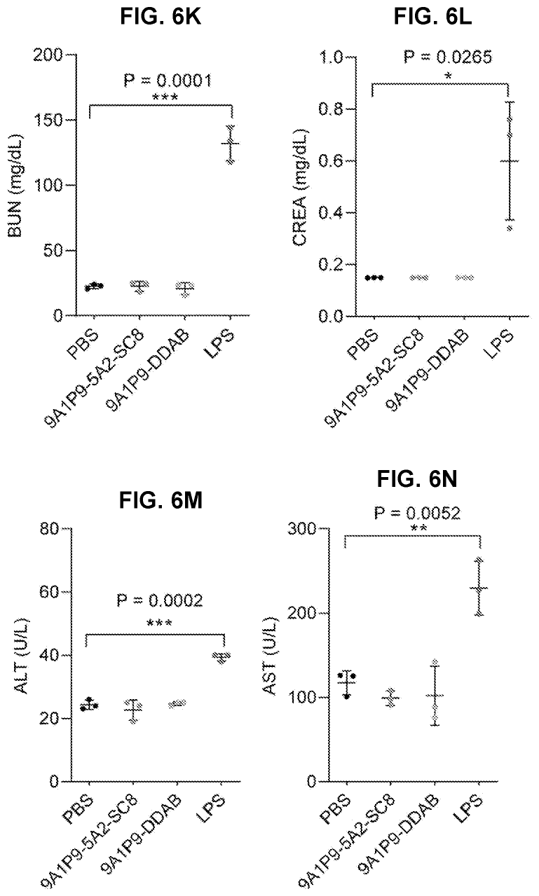

In accordance with the present disclosure, although LNPs have been utilized to deliver mRNA, there are still few reports for successful in vivo Cas9 mRNA/sgRNA delivery for CRISPR/Cas gene editing, and even less so with precision to specific organs.¹ Since the iPLNP system showed high mRNA delivery efficacy and organ selectivity, it was next utilized to co-deliver Cas9 mRNA and sgRNA for gene editing. 9A1P9-5A2-SC8 and 9A1P9-DDAB iPLNPs containing Cas9 mRNA and Tom1 sgRNA (sgTom1) with a 4:1 weight ratio were intravenously (IV) administered into Ai9 mice at a total RNA dose of 0.75 mg kg⁻¹, which would delete the stop cassettes and activate tdTomato protein (FIG. 6A). Fluorescent tdTomato protein was observed specifically in the liver after 9A1P9-5A2-SC8 iPLNP administration by ex vivo organ imaging (FIG. 6B). Sectioned organ analysis by confocal fluorescence microscopy showed tdTomato-positive cells in liver tissues (FIG. 6C). Similarly, 9A1P9-DDAB iPLNP induced specific gene editing in the lungs (FIGS. 6D-6E). Following this, PTEN sgRNA (sgPTEN) was co-delivered with Cas9 mRNA for gene editing in C57BL/6 mice (Cas9 mRNA/sgPTEN weight ratio, 4:1; total RNA dose, 0.75 mg kg⁻¹) targeting an endogenous gene. T7E1 assay showed efficient target gene editing in liver and lung by 9A1P9-5A2-SC8 and 9A1P9-DDAB iPLNPs, respectively (FIG. 6F). CRISPR/Cas9 gene editing in specific organs has remained a long-standing challenge in research and clinical translation. In this study, the highly efficient and organ selective gene editing broadened the iPLNP application to diverse genetic diseases.

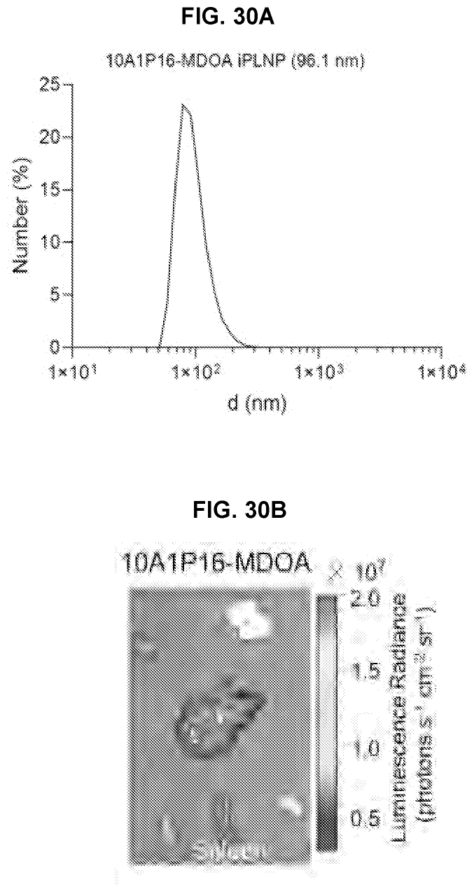

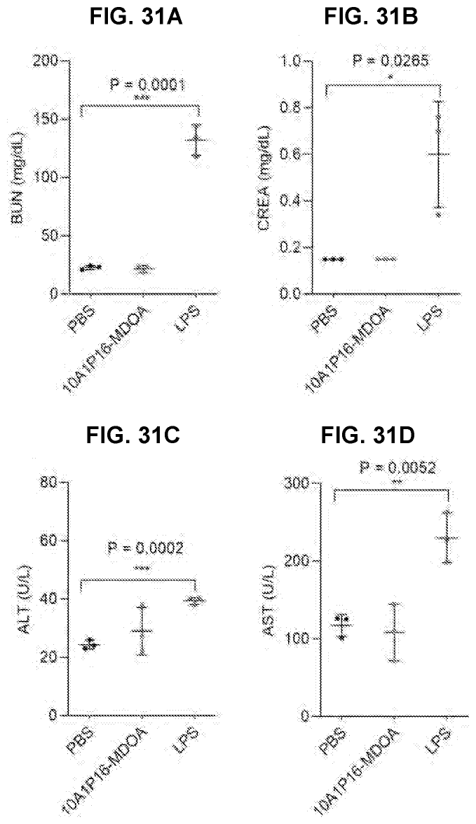

Considering potential pre-clinical activities, lead iPLNPs were manufactured at higher scale using controlled microfluidic mixing. Precise control over the mixing speed and volume ratios enabled the preparation of smaller 9A1P9-5A2-SC8 iPLNPs (77.2 nm, liver specific), 9A1P9-DDAB iPLNPs (108.1 nm, lung specific), and 10A1P16-MDOA iPLNPs (96.1 nm, spleen specific). Importantly, high in vivo mRNA delivery efficacy and precise organ selectivity were fully retained after decreasing iPLNP diameters (FIGS. 6G-6H and FIGS. 30A-30B). Furthermore, iPLNPs allowed repeat dosing, where high efficacy was retained after each repeat injection (FIGS. 6I-6J). Analysis of liver function enzymes and tissue section histology indicated that these iPLNPs showed negligible in vivo toxicity at the tested doses (FIGS. 6K-6N and FIGS. 31A-31D). These results highlight the potential of iPLNP system for future applications.

### Example 7. iPLNPs Achieved Nucleic Acid Delivery Following Subcutaneous Injection

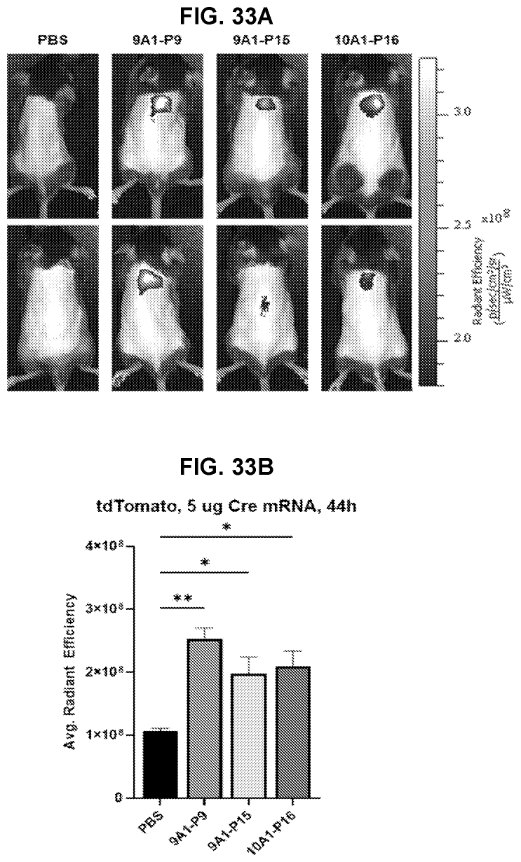

iPLNPs were formulated using the ethanol dilution method. The molar ratio of lipid components for each iPLNP was as follows: 25:30:30:1, iPhos lipid:Cholesterol:DODAP:PEG-DMG. The weight ratio of iPhos lipid to Cre Recombinase (CRE) mRNA was fixed at 18:1. 5 μg of CRE mRNA iPLNPs were injected subcutaneously into Ai9 mice. The mice were imaged for tdTomato signal using IVIS 44 hours after subcutaneous injection with CRE mRNA iPLNPs. 9A1-P9, 9A1-P15, and 10A1-P16 iPLNPs were able to deliver CRE mRNA to cells to achieve gene editing, wherein the DNA was edited to turn on expression of red florescence reporter tdTomato protein (FIGS. 33A-33B).

### Discussion of Examples 1-7

The CRISPR/Cas9 gene editing system is gaining increasing interest due to its tremendous potential for genetic disease treatment. Although cells build membranes and mediate transport using phospholipids, most all efficacious lipid nanoparticles for gene delivery rely on ionizable amines as the key physiochemical parameter to mediate endosomal escape via charge acquisition. In carrier development, synthetic zwitterionic lipids have been largely unexplored, even though they may readily enable endosomal membrane fusion and leakiness due to their homology with biological membranes. Although zwitterions have been reported to benefit stability, RNA encapsulation, cellular uptake, and pharmacokinetics of nanoparticles, current phospholipids are limited by lack of chemical architecture flexibility.

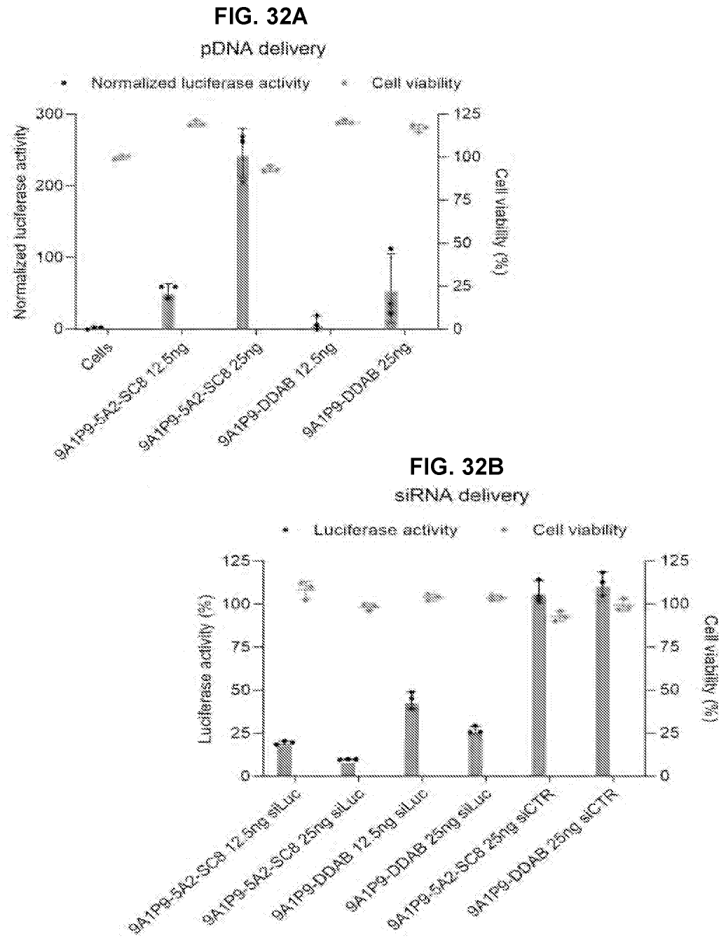

Therefore, the present disclosure aimed to develop new phospholipids using chemical synthesis, which revealed highly appealing candidates to insert into the biological membranes for efficient cargo escape from endosomes. Meanwhile, structures and functions of phospholipids could be well tailored, specifically allowing for acidic endosome rupture and preventing blood hemolytic effects at physiological environment. The rational design of iPhos lipids involved a pH-switchable small zwitterion head and a three tail body. This unique architecture makes it easy to insert into naturally occurring membrane phospholipids and induce phase transformation for RNA release from endosomes. SAR revealed that iPhos chain lengths can control in vivo mRNA delivery efficacy and organ selectivity. Moreover, diverse existing zwitterionic, ionizable cationic, and permanently cationic helper lipids were evaluated in our iPLNP system, which mediated mRNA translation selectively in spleen, liver, and lungs. Ultimately, the top 9A1P9-5A2-SC8 and 9A1P9-DDAB iPLNPs were utilized to co-deliver mRNA and sgRNA to edit reporter and endogenous genes, and long-term challenging organ selective CRISPR/Cas9 gene editing was achieved. Additionally, these iPLNPs showed broad applicability to deliver other nucleic acids, including plasmid DNA and siRNA (FIGS. 32A-32B). These profiles impart synthetic ionizable phospholipids great promise for the treatment of diverse genetic diseases with minimized side effects.

### Materials and Methods Used in Examples 1-7

Materials—Chemicals and Reagents for Synthesis. 2-Chloro-2-oxo-1,3,2-dioxaphospholane (COP) and triethylamine (TEA) were purchased from Fisher Scientific. Amines, alcohols, cholesterol (chol), and N-Methyldioctadecylamine (MDOA) were purchased from Sigma-Aldrich. 1,2-dioleoyl-sn-glycero-3-phosphoethanolamine (DOPE), 1,2-dioleoyl-sn-glycero-3-phospho-L-serine (sodium salt) (DOPS), 1,2-dioleoyl-sn-glycero-3-phosphocholine (DOPC), 1,2-dioleoyl-sn-glycero-3-phosphoethanolamine-N-(7-nitro-2-1,3-benzoxadiazol-4-yl) (ammonium salt) (NBD-PE), 1,2-dioleoyl-sn-glycero-3-phosphoethanolamine-N-(lissamine rhodamine B sulfonyl) (ammonium salt) (N—Rh-PE), 1,2-distearoyl-sn-glycero-3-phosphocholine (DSPC), 1,2-dioleoyl-3-dimethylammonium-propane (DODAP), 1,2-dioleoyl-3-trimethylammonium-propane (chloride salt) (DOTAP) and dimethyldioctadecylammonium (Bromide Salt) (DDAB) were purchased from Avanti Lipids. 1,2-dimyristoyl-rac-glycero-3-methoxy(poly(ethylene glycol))-2000 (DMG-PEG2000) was obtained from NOF America. The ionizable cationic lipid 5A2-SC8 was prepared as per our previous literature report. DLin-MC3-DMA was purchased from MedKoo Biosciences, and used as per the literature reported formulation details. Organic solvents were purchased from Sigma-Aldrich.

Materials—Reagents for Biological Assays. Dulbecco's modified phosphate buffered saline (PBS), RPMI-1640 medium, fetal bovine serum (FBS), and trypsin-EDTA (0.25%) were purchased from Sigma-Aldrich. Firefly luciferase messenger RNA (mRNA), Cre mRNA, and Cas9 mRNA were purchased from TriLink Biotechnologies. The Quant-iT RiboGreen RNA assay kit was purchased from Life Technologies. ONE-Glo+Tox luciferase assay kit was purchased from Promega. D-Luciferin firefly, sodium salt monohydrate was purchased from Gold Biotechnology.

Synthesis of alkylated dioxaphospholane oxide molecules P4-P16. P4-P16 were synthesized via esterification of 2-chloro-2-oxo-1,3,2-dioxaphospholane (COP) by corresponding alcohols with different alkyl chain length. For instance, to prepare P4, 1-butanol (30 mmol) and triethylamine (TEA, 30 mmol) were dissolved in 25 mL anhydrous tetrahydrofuran (THF). Then COP (30 mmol) solution in 10 mL THF was added dropwise to the mixture at −15° C. Afterward, the reaction continued at 25° C. for 12 h. The mixture was filtered to remove the triethylamine hydrochloride and the filtrate was concentrated by rotary evaporation to give P4. P5-P10 molecules were synthesized using respective alcohols and following the general protocol described above. For P11-P16 synthesis, COP was added to corresponding alcohols at 0° C., and other procedures were retained the same. All P4-P16 synthesis gave the yields over 90%.

General synthesis of ionizable phospholipids (iPhos) library. iPhos (nAxPm) were synthesized through orthogonal reactions by amines (1A-28A) and alkylated dioxaphospholane oxide molecules (Pm, m=4-16). “x” indicates the Pm molecule number modified on one amine molecule, and each Pm molecule could introduce one phosphate group and one hydrophobic alkyl chain into the iPhos. Each primary, secondary, or tertiary amine was designed to consume one equivalent of alkylated dioxaphospholane oxide molecules Pm. For amines nA (n=1-18) with a single primary, secondary or tertiary amine, 1.1 equivalents of Pm were reacted with amines to obtain nA1Pm. For nA (n=19-28) with multiple amine groups, each amine group was designed to introduce one zwitterion at the most. Briefly, the amines were reacted with 2.2 equivalents, 3.3 equivalents, 4.4 equivalents and 5.5 equivalents of Pm to give nA2Pm, nA3Pm, nA4Pm and nA5Pm iPhos, respectively. All the reactions were conducted in anhydrous dimethyl sulfoxide (DMSO) at the starting material concentration of 0.3 g/mL. The mixtures were stirred at 70° C. for 3 days, then DMSO was removed through vacuum drying.

Initial mRNA delivery (in vitro and in vivo screening) experiments were conducted using crude iPhos. Selected top iPhos (e.g. 9A1P9, 10A1P10, 9A1P15 and 10A1P16) were purified by column flash chromatography and used for additional characterizations (including size, zeta potential, mRNA binding, pKa, hemolysis, FRET studies, etc.) and in vivo evaluations. The products were eluted and fractionated with a solvent gradient of 3% chloroform in methanol to 10% chloroform in methanol. The final iPhos were concentrated by rotary evaporation and dried under vacuum for 24 h.

In vitro iPhos nanoparticle (iPLNP) formulation and characterization. iPLNPs were prepared by the ethanol dilution method. mRNA was diluted in citric acid/sodium citrate buffer (10 mM, pH 4.4). The lipid mixture containing synthetic iPhos, MDOA, cholesterol, and DMG-PEG2000 was prepared in ethanol. The two solutions were rapidly mixed by pipette at a 3:1 aqueous: ethanol volumetric ratio. Post incubation for 15 minutes, the nanoparticles were diluted 3-fold with 1×PBS buffer for in vitro mRNA delivery.

For particle size and Ribogreen mRNA binding measurement, the nanoparticles were diluted 5-fold with 1×PBS buffer. Zeta potential was recorded with nanoparticles diluted by 10-fold 1×PBS buffer. Zetasizer Nano ZS (Malvern) with a He—Ne laser (λ=632 nm) was used for particle size and zeta potential measurement. Particle sizes were measured by dynamic light scattering (DLS) and zeta potentials were determined by electrophoretic light scattering.

pKa determination using the 2-(p-toluidino)-6-naphthalenesulfonic acid (TNS) assay. The pKa of each iPLNP was determined by TNS assay. The iPLNPs comprised of synthetic iPhos/MDOA/chol/DMG-PEG2000 (25/30/30/1 mol %) were formulated in PBS at a concentration of 0.6 mM total lipid. TNS was prepared as a 100 μM stock solution in milliQ water. The nanoparticles were diluted to 6 μM total lipid in 100 μL volume per well in 96-well plates with buffer solutions containing 10 mM 4-(2-hydroxyethyl)-1-piperazineethanesulfonic acid (HEPES), 10 mM 4-morpholineethanesulfonic acid (MES), 10 mM ammonium acetate and 130 mM NaCl, where the pH ranged from 2.5 to 11. The TNS stock solution was added into each well to give a final concentration of 5 μM. Afterward, the plate was read using excitation and emission wavelength of 321 nm and 445 nm, respectively. A sigmoidal fit analysis was applied to the fluorescence data and the pKa was measured as the pH giving rise to half-maximal fluorescence intensity.

Hemolysis assay. Mouse red blood cells (RBCs) were isolated from freshly collected whole blood by centrifuging at 10,000×g for 5 min, then the RBCs were washed 5 times with PBS buffer (pH 7.4). Afterward, RBCs were suspended in PBS of pH 7.4 and 5.5, respectively. iPLNPs were formulated using the in vitro iPLNP formulation method outlined above. Lipids were dissolved in chloroform, rotary evaporated, and vacuum dried for another 2 h to give a thin lipid film. Then, PBS (pH 7.4) was added and sonicated for 20 min to obtain particle suspension. RBC suspension was added to 96-well plates, and calculated iPLNPs or lipids were added to the wells. After incubating at 37° C. for 1 h, the RBC solutions were centrifuged at 10,000×g for 5 min and the supernatants containing hemoglobin were collected. The hemoglobin contents were evaluated at a wavelength of 540 nm with a microplate reader. That RBC suspension incubated in PBS was set as negative control, and RBC suspension incubated in Triton-X solutions (1 wt. %) was set as positive control.

Lipid mixing and fusion characterization by fluorescence resonance energy transfer (FRET) assay. Lipid mixing and fusion with endosome mimicking anionic liposomes were determined by a FRET assay. The DOPE-conjugated FRET probes NBD-PE and N—Rh-PE were formulated into the same endosome mimicking nanoparticle, leading to attenuated NBD fluorescence because of FRET to rhodamine. Once lipid fusion occurred, NBD signal would increase due to the larger distance between the two probes. Endosome mimicking anionic liposomes were prepared by mixing DOPS: DOPC: DOPE: NBD-PE: N—Rh-PE (molar ratio 25:25:48:1:1) in chloroform, followed by rotary evaporation and another 2 h vacuum dry to give a thin lipid film. The dried film was subsequently resuspended in PBS (pH 7.4) by sonicating for 20 min and total lipid concentration was fixed at 1 mM. iPLNPs were formulated using the in vitro iPLNP formulation method outlined above with an iPhos concentration of 1 mM. iPhos 10A1P10 was dissolved in chloroform, and rotary evaporated to give a thin lipid film. Then, PBS (pH 7.4) was added and sonicated for 20 min to obtain particle suspension (10 mM). 25A3P10 with multiple zwitterions failed to form particle suspension after sonication, so only lipid fusion of 25A3P10 iPLNPs was evaluated. PBS (pH 5.5) was added to black 96-well plates (100 μL/well), and 1 μL of endosome mimicking anionic liposomes (1 mM) was added to each well. Then 10 μL iPLNPs or 1 μL lipid suspensions were added to the wells. After incubating at 37° C. for 5 min, fluorescence measurements (F) were conducted on a microplate reader at Ex/Em=465/520 nm. Only endosome mimicking anionic liposomes in PBS were set as negative control (Fₘᵢₙ). Lipids containing probes incubated with Triton-X solutions (2 wt. %) were set as positive control (Fₘₐₓ). The lipid fusion (%) was calculated as (F−Fₘᵢₙ)/(Fₘₐₓ−Fₘᵢₙ)*100%.

iPLNP dissociation by FRET assay. iPLNP dissociation was measured by mixing iPLNPs and endosome mimicking anionic liposomes. The DOPE-conjugated FRET probes NBD-PE and N—Rh-PE were formulated into the same iPLNP. iPLNPs were prepared using iPhos: MDOA: chol: DMG-PEG2000: NBD-PE: N—Rh-PE lipid mixtures (molar ratio 25:30:30:1:0.86:0.86), with a final total lipid concentration of 1 mM. Other procedures were the same as the in vitro iPLNP formulation method outlined above. Endosome mimicking anionic liposomes were prepared by mixing DOPS: DOPC: DOPE (molar ratio 25:25:50) in chloroform, followed by rotary evaporation and another 2 h vacuum dry to give a thin lipid film. The dried film was subsequently resuspended in PBS (pH 7.4) by sonicating for 20 min and total lipid concentration was fixed at 10 mM. PBS (pH 5.5) was added to black 96-well plates (100 μL/well), and 1 μL iPLNPs were added to each well. Then 1 μL endosome mimicking anionic liposomes were added to the wells. After incubating at 37° C. for 10 min (or other noted time intervals), fluorescence measurements (F) were conducted on a microplate reader at Ex/Em=465/520 nm. iPLNPs (with NBD-PE and N—Rh-PE inside) in PBS were set as negative control (Fₘᵢₙ). iPLNPs (with NBD-PE and N—Rh-PE inside) incubated with Triton-X solutions (2 wt. %) were set as positive control (Fₘₐₓ). The iPLNP dissociation (%) was calculated as (F−Fₘᵢₙ)/(Fₘₐₓ−Fₘᵢₙ)*100%.

Cell culture. Human ovarian adenocarcinoma cells (IGROV1) was cultured in RPMI-1640 medium with 10% FBS and 1% Penicillin/Streptomycin (P/S). Cells were cultured at 37° C. and 5% CO₂ in a humidified atmosphere.

In vitro screening of iPhos for mRNA delivery. IGROV1 cells were seeded at a density of 1×10⁴ cells per well (100 μL RPMI-1640 medium supplied with 10% FBS and 1% P/S) in white opaque 96-well plates. 24 h later, nanoparticles with Fluc mRNA were prepared using the in vitro iPLNP formulation method outlined above in 96-well plates by rapid mixing of aqueous phase and ethanol phase (v/v=3:1) with a multichannel pipette. iPLNPs were prepared at synthetic iPhos: mRNA molar ratio of 11622:1 and lipid mixture synthetic iPhos: MDOA: chol: DMG-PEG2000 molar ratio of 25:30:30:1. The synthetic iPhos: mRNA molar ratio of 11622:1 was fixed, where 10A1P4-12A1P16: mRNA showed an average weight ratio of 10±2.5. This could ensure that each iPLNP had same moles of lipid mixture. The ratios were also used for other characterizations and in vivo evaluation, unless other noted. 50 ng mRNA per well was used. Then, 150 μL fresh cell culture media were utilized to replace the previous media, and the formulated iPLNPs were added into the cells. Post another 24 h incubation, luciferase expression and cell viability were evaluated with the ONE-Glo 30 Tox luciferase assay kits. All transfection assays were performed in triplicate, and the average with standard deviation was reported.

In vivo iPLNP formulation and characterization. The mRNA was diluted in citric acid/sodium citrate buffer (10 mM, pH 3.2). The lipid mixture containing synthetic iPhos, MDOA, cholesterol, and DMG-PEG2000 was prepared in ethanol. The two phases were rapidly mixed by pipette at a 3:1 aqueous: ethanol volumetric ratio. Post incubation for 15 minutes, iPLNPs were dialyzed against 1×PBS in Pur-A-Lyzer midi dialysis chambers (Sigma-Aldrich) for in vivo use.

Animal experiments. All experiments were approved by the Institutional Animal Care and Use Committees of The University of Texas Southwestern Medical Center and were consistent with local, state, and federal regulations as applicable. Female C57BL/6 mice were purchased from the UT Southwestern animal breeding core. B6.Cg-Gt(ROSA)26Sor^{tm9(CAG-dTomato)Hze}/J mice (also known as Ai9 or Ai9(RCL-tdT) mice) were obtained from The Jackson Laboratory (007909) and bred to maintain homozygous expression of the Cre reporter allele that has a LoxP-flanked STOP cassette preventing transcription of a CAG promoter-driven red fluorescent tdTomato protein. Following Cre-mediated recombination, Ai9 mice will express tdTomato fluorescence. Ai9 mice are congenic on the C57BL/6J genetic background.

In vivo luciferase mRNA delivery. For iPhos in vivo screening, nanoparticles containing Fluc mRNA were prepared as in vivo iPLNP formulation method mentioned above. Unless otherwise noted, the ratios of formulations were in accordance with that of the in vitro screening. Briefly, iPLNPs were prepared at synthetic iPhos: Fluc mRNA molar ratio of 11622:1 and lipid mixture synthetic iPhos: MDOA: chol: DMG-PEG2000 molar ratio of 25:30:30:1. Then, the nanoparticles were administered to female C57BL/6 mice (6-8 week old) via intravenous (IV) injection. 6 h later, the luciferase expression was evaluated by live animal bioluminescence imaging. Briefly, mice were anesthetized under isofluorane, and 100 μL of D-luciferin (GoldBio, 30 mg/mL in PBS) substrate was intraperitoneally injected. After 5 minutes under anesthesia, the luciferase activity was imaged on an IVIS Lumina system (Perkin Elmer). Afterward, the organs were isolated and imaged with the same method. The images were processed with the Living Image analysis software (Perkin Elmer).

iPhos 9A1P9 was used to compare with commercial phospholipids DOPE and DSPC. C57BL/6 mice were IV injected by nanoparticles at 0.25 mg/kg FLuc mRNA and luminescence was quantified 6 h post injection. 9A1P9: MDOA: chol: DMG-PEG2000 molar ratio of 25:30:30:1 and 9A1P9/mRNA weight ratio of 18:1 were used. For commercial phospholipid comparison, equimolar DOPE or DSPC were utilized to replace 9A1P9. Other procedures were conducted the same as mentioned above.

For 9A1P9 iPLNPs with different helper lipids, 9A1P9: DOPE: chol: DMG-PEG2000 (molar ratio) of 55:30:45:0.2, 9A1P9: MDOA (DODAP or 5A2-SC8): chol: DMG-PEG2000 (molar ratio) of 25:30:30:1 and 9A1P9: DDAB (or DOTAP): chol: DMG-PEG2000 (molar ratio) of 60:30:40:0.4 were used. For all the formulations, 9A1P9: mRNA weight ratio was fixed at 18:1. Other procedures were conducted the same as mentioned above.

In vivo Cre mRNA delivery. Nanoparticles containing Cre mRNA were prepared as in vivo iPLNP formulation method mentioned above. Afterward, the nanoparticles were administered to Ai9 mice via IV injection. 48 h later, mice were sacrificed, and the organs were isolated and imaged on the IVIS Spectrum in vivo imaging system (Perkin Elmer).

In vivo biodistribution. Nanoparticles containing Cy5-labeled Fluc mRNA (Cy5-mRNA, 0.25 mg/kg) were prepared as in vivo iPLNP formulation method mentioned above. iPLNPs were administered to female C57BL/6 mice (6-8 week old) via IV injection. 6 h later, mice were sacrificed, and the organs were isolated and imaged on the IVIS Spectrum in vivo imaging system (Perkin Elmer).

In vivo co-delivery of Cas9 mRNA and sgTom1 for gene editing. Nanoparticles containing Cas9 mRNA and modified sgTom1 (mRNA/sgRNA weight ratio 4:1, total RNA dose 0.75 mg/kg) were prepared as in vivo iPLNP formulation method mentioned above. 9A1P9: 5A2-SC8: chol: DMG-PEG2000 (molar ratio) of 25:30:30:1 and 9A1P9: DDAB: chol: DMG-PEG2000 (molar ratio) of 60:30:40:0.4 were used for 9A1P9-5A2-SC8 iPLNP and 9A1P9-DDAB iPLNP, respectively. 9A1P9/RNA weight ratio was fixed at 18:1. Afterward, iPLNPs were administered to Ai9 mice via IV injection. PBS group was used as negative control. 10 days later, mice were sacrificed and the organs were isolated and imaged on the IVIS Spectrum in vivo imaging system (Perkin Elmer). Then, tissues were embedded in optimal cutting temperature (OCT) compound and cut into 10 μm sections. These sections were fixed with 4% paraformaldehyde for 20 min at RT and washed three times with PBS. Afterward, one drop of ProLong Gold Mountant with DAPI was applied and coverslips were covered on these slides. Then these slides were imaged by confocal microscopy (Zeiss LSM 700).

In vivo co-delivery of Cas9 mRNA and sgPTEN for gene editing in C57BL/6 mice. PTEN was selected to examine endogenous gene editing in vivo. iPLNPs containing Cas9 mRNA and modified sgPTEN (mRNA/sgRNA weight ratio 4:1, total RNA dose 0.75 mg/kg) were prepared as in vivo iPLNP formulation method mentioned above. 9A1P9: 5A2-SC8: chol: DMG-PEG2000 (molar ratio) of 25:30:30:1 and 9A1P9: DDAB: chol: DMG-PEG2000 (molar ratio) of 60:30:40:0.4 were used for 9A1P9-5A2-SC8 iPLNP and 9A1P9-DDAB iPLNP, respectively. 9A1P9/RNA weight ratio was fixed at 18:1. Afterward, iPLNPs were administered to wild type C57BL/6 mice (6-8 week old) via IV injection. 10 days later, tissues were collected, and genomic DNA was extracted with a PureLink Genomic DNA Mini Kit (ThermoFisher). After obtaining PTEN PCR products, the T7E1 assay (NEB) was performed to confirm gene editing efficacy by the standard protocol. Furthermore, gene editing efficacy of PTEN was evaluated by Image J based on the following formula: Indel (%)=100×(1−(1−fraction cleaved)\hat{ }0.5), where the fraction cleaved=(Fragment 1+Fragment 2)/(Fragment 1+Fragment 2+Parent Fragment). sgRNA sequences and PCR primers used herein are provided in Table 2 and Table 3, respectively.

TABLE 2
sgRNA sequences

| | Target Sequences | PAM |
|---|---|---|
| Name | (5′ to 3′) | (5′ to 3′) |
| sgTOM | AAGTAAAACCTCTACAAATG | TGG |
| | (SEQ ID NO: 1) | |
| sgPTEN | AGATCGTTAGCAGAAACAAA | AGG |
| | (SEQ ID NO: 2) | |

TABLE 3
PCR primers

| Name | Sequence (5′ to 3′) | Length | Purpose |
|---|---|---|---|
| PTEN_ | AAGCAGGCCCAGTCTCTG | 582 bp | For T7E1 |
| Forward | (SEQ ID NO: 3) | | assay |
| PTEN_ | GACGAGCTCGCTAATCCA | | |
| Reverse | GTG (SEQ ID NO: 4) | | |

Statistical analyses. Statistical analyses were performed using GraphPad Prism version 7 (GraphPad Software). Two-tailed un-paired Student's t-test was used to compare two groups and one-way ANOVA was utilized to compare multiple replicate groups. P-values<0.05 (*), P<0.01 (**) and P<0.001 (***) were considered to be statistically significant.

### Citations Used in Examples 1-7

1. Wang, H. X. et al. CRISPR/Cas9-based genome editing for disease modeling and therapy: Challenges and opportunities for nonviral delivery. Chem. Rev. 117, 9874-9906 (2017).
2. Hajj, K. A. & Whitehead, K. A. Tools for translation: Non-viral materials for therapeutic mRNA delivery. Nat. Rev. Mater. 2, 17056 (2017).
3. Jinek, M. et al. A programmable dual-RNA-guided DNA endonuclease in adaptive bacterial immunity. Science 337, 816-821 (2012).
4. Cong, L. et al. Multiplex genome engineering using CRISPR/Cas systems. Science 339, 819-823 (2013).
5. Mali, P. et al. RNA-guided human genome engineering via Cas9. Science 339, 823-826 (2013).
6. Yin, H. et al. Structure-guided chemical modification of guide RNA enables potent non-viral in vivo genome editing. Nat. Biotechnol. 35, 1179 (2017).
7. Liu, S. et al. Highly branched poly(β-amino ester) delivery of minicircle DNA for transfection of neurodegenerative disease related cells. Nat. Commun. 10, 3307 (2019).
8. Zhou, J. et al. Biodegradable poly(amine-co-ester) terpolymers for targeted gene delivery. Nat. Mater. 11, 82-90 (2012).
9. Liu, S. et al. Bioreducible zinc(II)-coordinative polyethylenimine with low molecular weight for robust gene delivery of primary and stem cells. J. Am. Chem. Soc. 139, 5102-5109 (2017).
10. Kanasty, R., Dorkin, J. R., Vegas, A. & Anderson, D. Delivery materials for siRNA therapeutics. Nat. Mater. 12, 967-977 (2013).
11. Semple, S. C. et al. Rational design of cationic lipids for siRNA delivery. Nat. Biotechnol. 28, 172-176 (2010).
12. Cheng, Q. et al. Dendrimer-based lipid nanoparticles deliver therapeutic FAH mRNA to normalize liver function and extend survival in a mouse model of hepatorenal tyrosinemia type I. Adv. Mater. 30, e1805308 (2018).
13. van Meer, G., Voelker, D. R. & Feigenson, G. W. Membrane lipids: Where they are and how they behave. Nat. Rev. Mol. Cell Biol. 9, 112-124 (2008).
14. Sahay, G., Alakhova, D. Y. & Kabanov, A. V. Endocytosis of nanomedicines. J. Controlled Release 145, 182-195 (2010).
15. Gilleron, J. et al. Image-based analysis of lipid nanoparticle-mediated siRNA delivery, intracellular trafficking and endosomal escape. Nat. Biotechnol. 31, 638-646 (2013).
16. Sahay, G. et al. Efficiency of siRNA delivery by lipid nanoparticles is limited by endocytic recycling. Nat. Biotechnol. 31, 653-658 (2013).
17. Wittrup, A. et al. Visualizing lipid-formulated siRNA release from endosomes and target gene knockdown. Nat. Biotechnol. 33, 870-876 (2015).
18. Miller, J. B. & Siegwart, D. J. Design of synthetic materials for intracellular delivery of RNAs: From siRNA-mediated gene silencing to CRISPR/Cas gene editing. Nano Res. 11, 5310-5337 (2018).
19. Truong, B. et al. Lipid nanoparticle-targeted mRNA therapy as a treatment for the inherited metabolic liver disorder arginase deficiency. Proc. Natl. Acad. Sci. U.S.A. 116, 21150-21159 (2019).
20. Mout, R., Ray, M., Lee, Y. W., Scaletti, F. & Rotello, V. M. In vivo delivery of CRISPR/Cas9 for therapeutic gene editing: Progress and challenges. Bioconjugate Chem. 28, 880-884 (2017).
21. Luther, D. C., Lee, Y. W., Nagaraj, H., Scaletti, F. & Rotello, V. M. Delivery approaches for CRISPR/Cas9 therapeutics in vivo: Advances and challenges. Expert Opin. Drug Deliv. 15, 905-913 (2018).
22. Miller, J. B. et al. Non-viral CRISPR/Cas gene editing in vitro and in vivo enabled by synthetic nanoparticle co-delivery of Cas9 mRNA and sgRNA. Angew. Chem. Int. Ed. 56, 1059-1063 (2017).
23. Alabi, C. A. et al. Multiparametric approach for the evaluation of lipid nanoparticles for siRNA delivery. Proc. Natl. Acad. Sci. U.S.A. 110, 12881-12886 (2013).
24. Akinc, A. et al. A combinatorial library of lipid-like materials for delivery of RNAi therapeutics. Nat. Biotechnol. 26, 561-569 (2008).
25. Love, K. et al. Lipid-like materials for low-dose, in vivo gene silencing. Proc. Natl. Acad. Sci. U.S.A. 107, 1864-1869 (2010).
26. Zhou, K. et al. Modular degradable dendrimers enable small RNAs to extend survival in an aggressive liver cancer model. Proc. Natl. Acad. Sci. U.S.A. 113, 520-525 (2016).
27. Wei, T. et al. Anticancer drug nanomicelles formed by self-assembling amphiphilic dendrimer to combat cancer drug resistance. Proc. Natl. Acad. Sci. U.S.A. 112, 2978-2983 (2015).
28. Zhang, Y. et al. The development of an in vitro assay to screen lipid based nanoparticles for siRNA delivery. J. Controlled Release 174, 7-14 (2014).
29. Cheng, Y., Yumul, R. C. & Pun, S. H. Virus-inspired polymer for efficient in vitro and in vivo gene delivery. Angew. Chem. Int. Ed. 55, 12013-12017 (2016).
30. Zhou, D. et al. The transition from linear to highly branched poly(beta-amino ester)s: Branching matters for gene delivery. Science Adv. 2, e1600102 (2016).
31. Li, B. et al. An orthogonal array optimization of lipid-like nanoparticles for mRNA delivery in vivo. Nano Lett. 15, 8099-8107 (2015).

What is claimed is:

1. A synthetic ionizable phospholipid comprising Formula (I): 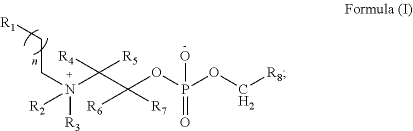
wherein:
R₁ is selected from the group consisting of C2-C20 unsubstituted alkyl; C2-C20 substituted alkyl, C2-C20 unsubstituted alkenyl, C2-C20 substituted alkenyl, C2-C20 unsubstituted alkynyl, C2-C20 substituted alkynyl, C4-C20 unsubstituted cycloalkyl, and C4-C20 substituted cycloalkyl;
R₂, and R₃ are independently selected from the group consisting of H, C1-C20 unsubstituted alkyl, C1-C20 substituted alkyl, C1-C20 unsubstituted alkenyl, C1-C20 substituted alkenyl, C1-C20 unsubstituted alkynyl, C1-C20 substituted alkynyl, C4-C20 unsubstituted cycloalkyl, and C4-C20 substituted cycloalkyl;
R₄, R₅, R₆, and R₇ are independently selected from the group consisting of H, C1-C8 unsubstituted alkyl, C1-C8 substituted alkyl, C1-C8 unsubstituted alkenyl, C1-C8 substituted alkenyl, C1-C8 unsubstituted alkynyl, and C1-C8 substituted alkynyl;
R₈ is selected from the group consisting of C3-C21 unsubstituted alkyl, C3-C21 substituted alkyl, C3-C21 unsubstituted alkenyl, C3-C21 substituted alkenyl, C3-C21 unsubstituted alkynyl, and C3-C21 substituted alkynyl; and
n is an integer from 1 to 4.

2. The synthetic ionizable phospholipid according to claim 1,
wherein R₁, is selected from a group consisting of C2-C16 unsubstituted alkyl; C2-C16 substituted alkyl, or C4-C12 substituted cycloalkyl;
R₂, and R₃ are independently selected from a group consisting of H, C1-C16 unsubstituted alkyl, C1-C16 substituted alkyl, or C4-C16 substituted cycloalkyl;
R₄, R₅, R₆, and R₇ are independently selected from a group consisting of H, C1-C4 unsubstituted alkyl, or C1-C4 substituted alkyl;
R₈ is selected from C3-C18 unsubstituted alkyl; and
n is an integer from 1 to 3.

3. The synthetic ionizable phospholipid to claim 1,
wherein R₁ is C2-C15 unsubstituted alkyl;
R₂ and R₃ are independently selected from a group consisting of H, C1-C16 substituted alkyl, or C4-C16 substituted cycloalkyl;
R₄, R₅, R₆, and R₇ are independently selected from a group consisting of H, methyl, or ethyl;
R₈ is selected from C4-C16 unsubstituted alkyl; and
n is an integer from 1 to 2.

4. A synthetic ionizable phospholipid comprising Formula (II): 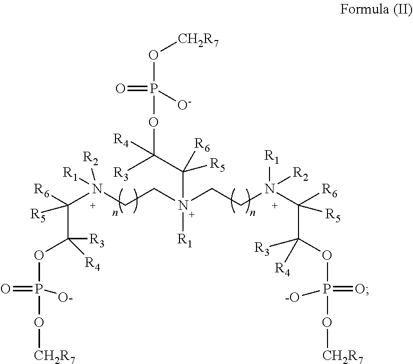
wherein R₁ and R₂ are independently selected from a group consisting of H, C1-C8 substituted alkyl, or C1-C8 unsubstituted alkyl;
R₃, R₄, R₅, and R₆ are independently selected from a group consisting of H, C1-C8 unsubstituted alkyl, or C1-C8 substituted alkyl;
R₇ is selected from a group consisting of C3-C21 unsubstituted alkyl or C3-C21 substituted alkyl; and
n is an integer from 1 to 4.

5. The synthetic ionizable phospholipid according to claim 4,
wherein R₁ and R₂ are independently selected from a group consisting of H, C1-C6 substituted alkyl, or C1-C6 unsubstituted alkyl;
R₃, R₄, R₅, and R₆ are independently selected from a group consisting of H, C1-C4 unsubstituted alkyl, or C1-C4 substituted alkyl;
R₇ is selected from a group consisting of C3-C18 unsubstituted alkyl or C3-C18 substituted alkyl; and
n is an integer from 1 to 3.

6. The synthetic ionizable phospholipid according to claim 4,
wherein R₁ and R₂ are independently selected from a group consisting of H, C1-C4 substituted alkyl, or C1-C4 unsubstituted alkyl;
R₃, R₄, R₅, and R₆ are independently selected from a group consisting of H, methyl, or ethyl;
R₇ is selected from a group consisting of C3-C15 unsubstituted alkyl or C3-C15 substituted alkyl; and
n is an integer from 1 to 2.

7. An ionizable phospholipid comprising Formula (III): 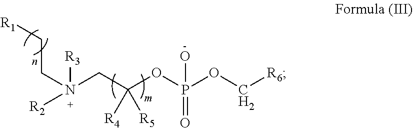
wherein:
R₁ is selected from a group consisting of C2-C20 unsubstituted alkyl; C2-C20 substituted alkyl, C2-C20 unsubstituted alkenyl, C2-C20 substituted alkenyl, C2-C20 unsubstituted alkynyl, C2-C20 substituted alkynyl, C4-C20 unsubstituted cycloalkyl, or C4-C20 substituted cycloalkyl;
R₂, and R₃ are independently selected from a group consisting of H, C1-C20 unsubstituted alkyl, C1-C20 substituted alkyl, C1-C20 unsubstituted alkenyl, C1-C20 substituted alkenyl, C1-C20 unsubstituted alkynyl, C1-C20 substituted alkynyl, C4-C20 unsubstituted cycloalkyl, or C4-C20 substituted cycloalkyl;
R₄ and R₅ are independently selected from a group consisting of H, C1-C8 unsubstituted alkyl, C1-C8 substituted alkyl, C1-C8 unsubstituted alkenyl, C1-C8 substituted alkenyl, C1-C8 unsubstituted alkynyl, or C1-C8 substituted alkynyl;
R₆ is selected from a group consisting of C3-C21 unsubstituted alkyl, C3-C21 substituted alkyl, C3-C21 unsubstituted alkenyl, C3-C21 substituted alkenyl, C3-C21 unsubstituted alkynyl, or C3-C21 substituted alkynyl;
n is an integer from 1 to 4; and
m is an integer from 1 to 4.

8. The ionizable phospholipid according to claim 7, wherein R₁ is selected from a group consisting of C2-C16 unsubstituted alkyl; C2-C16 substituted alkyl, C2-C16 unsubstituted alkenyl, C2-C16 substituted alkenyl, C2-C16 unsubstituted alkynyl, C2-C16 substituted alkynyl, C4-C16 unsubstituted cycloalkyl, or C4-C16 substituted cycloalkyl;
R₂, and R₃ are independently selected from a group consisting of H, C1-C16 unsubstituted alkyl, C1-C16 substituted alkyl, C1-C16 unsubstituted alkenyl, C1-C16 substituted alkenyl, C1-C16 unsubstituted alkynyl, C1-C16 substituted alkynyl, C4-C16 unsubstituted cycloalkyl, or C4-C16 substituted cycloalkyl;
R₄ and R₅ are independently selected from a group consisting of H, C1-C6 unsubstituted alkyl, C1-C6 substituted alkyl, C1-C6 unsubstituted alkenyl, C1-C6 substituted alkenyl, C1-C6 unsubstituted alkynyl, or C1-C6 substituted alkynyl;
R₆ is selected from a group consisting of C3-C18 unsubstituted alkyl, C3-C18 substituted alkyl, C3-C18 unsubstituted alkenyl, C3-C18 substituted alkenyl, C3-C18 unsubstituted alkynyl, or C3-C18 substituted alkynyl;
n is an integer from 1 to 3; and
m is an integer from 1 to 3.

9. A synthetic ionizable phospholipid comprising at least one phosphate group and at least one zwitterion, wherein the at least one zwitterion comprises a pH switchable zwitterion.

10. The synthetic ionizable phospholipid according to claim 9, further comprising a hydrophobic domain.

11. The synthetic ionizable phospholipid according to claim 9 or claim 10, further comprising one or more hydrophobic tails.

12. The synthetic ionizable phospholipid according to any one of claims 9-11, further comprising at least one tertiary amine.

13. The synthetic ionizable phospholipid according to claims 9-12, wherein the one or more hydrophobic tails consists of one hydrophobic tail to 10 hydrophobic tails.

14. The synthetic ionizable phospholipid according to any one of claims 9-13, wherein each of the one or more hydrophobic tails is an alkyl tail comprising an alkyl chain length of from 8 carbons to 16 carbons, from 8 carbons to 10 carbons, from 9 carbons to 12 carbons, from 13 carbons to 16 carbons, from 8 carbons to 16 carbons, or any combination thereof.

15. The synthetic ionizable phospholipid according to any one of claims 9-13, wherein at least one of the one or more hydrophobic tails is an alkyl tail comprising an alkyl chain length of from 8 carbons to 16 carbons, from 8 carbons to 10 carbons, from 9 carbons to 12 carbons, from 13 carbons to 16 carbons, from 8 carbons to 16 carbons, or any combination thereof.

16. A pharmaceutical composition comprising any one of the synthetic ionizable phospholipids according to claims 1-15.

17. The composition according to claim 16, further comprising a helper lipid.

18. The composition according to claim 17, wherein the helper lipid is selected from the group consisting of zwitterionic helper lipids, ionizable cationic helper lipids, and permanently cationic helper lipids.

19. The composition according to claim 17, wherein the helper lipid is selected from the group consisting of 1,2-dioleoyl-sn-glycero-3-phosphoethanolamine (DOPE), N-methyldioctadecylamine (MDOA), 1,2-dioleoyl-3-dimethylammonium-propane (DODAP), dimethyldioctadecylammonium bromide salt (DDAB), 1,2-dioleoyl-3-trimethylammonium-propane (DOTAP), and any combination thereof.

20. The composition according to any one of claims 16-19, further comprising further comprising cholesterol or a cholesterol derivative.

21. The composition according to any one of claims 16-20, further comprising 1,2-dimyristoyl-rac-glycero-3-methoxy(poly(ethylene glycol-2000)) (DMG-PEG2000).

22. The composition according to any one of claims 16-21, further comprising one or more multi-tailed ionizable phospholipids, 1,2-dioleoyl-sn-glycero-3-phosphoethanolamine (DOPE), and cholesterol in a 55:30:45 molar ratio.

23. The composition according to any one of claims 16-21, further comprising one or more multi-tailed ionizable phospholipids, N-methyldioctadecylamine (MDOA), and cholesterol in a 25:30:30 molar ratio.

24. The composition according to any one of claims 16-21, further comprising one or more multi-tailed ionizable phospholipids, 1,2-dioleoyl-3-dimethylammonium-propane (DODAP), and cholesterol in a 25:30:30 molar ratio.

25. The composition according to any one of claims 16-21, further comprising one or more multi-tailed ionizable phospholipids, 5A2-SC8, and cholesterol in a 25:30:30 molar ratio.

26. The composition according to any one of claims 16-21, further comprising one or more multi-tailed ionizable phospholipids, dimethyldioctadecylammonium bromide salt (DDAB), and cholesterol in a 60:30:40 molar ratio.

27. The composition according to any one of claims 16-21, further comprising one or more multi-tailed ionizable phospholipids, 1,2-dioleoyl-3-trimethylammonium-propane (DOTAP), and cholesterol in a 60:30:40 molar ratio.

28. The composition according to any one of claims 16-27, further comprising a cargo.

29. The composition according to claim 28, wherein the cargo is selected from the group consisting of an active pharmaceutical ingredient, a nucleic acid, mRNA, sgRNA, a CRISPR/Cas9 DNA sequence, a zinc finger nuclease (ZFN), transcription activator-like effector nucleases (TALENs), siRNA, miRNA, tRNA, ssDNA, base editors, peptides, proteins, CRISPR/Cas ribonucleoprotein (RNP) complexes, and any combination thereof.

30. The composition according to any one of claims 16-29, formulated for parenteral administration, intravenous administration, oral administration, topical administration, or any combination thereof.

31. A pharmaceutical composition, the composition comprising:
a lipid nanoparticle (LNP) loaded with a cargo,
wherein the LNP comprises:
an ionizable phospholipid according to any one of claims 1-15; or
one or more multi-tailed ionizable phospholipids, the one or more multi-tailed ionizable phospholipids comprising a pH-switchable zwitterion and three hydrophobic tails.

32. The pharmaceutical composition according to any one of claims 16-31, wherein the cargo is disposed within a core of the LNP.

33. The pharmaceutial composition according to any one of claims 16-31, wherein the one or more multi-tailed ionizable phospholipids form a nanoparticle structure substantially encapsulating the cargo.

34. The pharmaceutical composition according to any one of claims 16-33, wherein the cargo is selected from the group consisting of an active pharmaceutical ingredient, a nucleic acid, mRNA, sgRNA, a CRISPR/Cas9 DNA sequence, a zinc finger nuclease (ZFN), transcription activator-like effector nucleases (TALENs), siRNA, miRNA, tRNA, ssDNA, base editors, peptides, proteins, cirRNA, CRISPR/Cas ribonucleoprotein (RNP) complexes, and any combination thereof.

35. A method of delivering an active pharmaceutical ingredient to a subject, the method comprising administering to the subject a therapeutically effective amount of a pharmaceutical composition according to any one of claims 16-34, wherein the cargo is an active pharmaceutical ingredient.

36. A method of in vivo delivery of mRNA or mRNA/sgRNA for gene editing in a subject, the method comprising administering to the subject a therapeutically effective amount of a pharmaceutical composition according to any one of claims 16-34.

37. A method of causing selective protein expression in the spleen, liver, and/or lung of a subject, the method comprising administering to the subject a therapeutically effective amount of a pharmaceutical composition according to any one of claims 16-34, wherein the cargo is mRNA.

38. A method comprising administering to a subject, a pharmaceutical composition according to any one of claims 13-34 for gene delivery, gene editing, drug delivery, mRNA delivery, CRISPR/Cas9 gene editing, zinc finger nuclease (ZFN) gene editing, base editor gene editing, transcription activator-like effector nucleases (TALENs) gene editing in the subject.

39. A method for tissue-specific cargo delivery in a subject, the method comprising administering to the subject a therapeutically effective amount of a pharmaceutical composition according to any one of claims 16-34.

40. The method of claim 39, wherein the cargo comprises mRNA, CRISPR/Cas, or any combination thereof.
